(19) United States
(12) Patent Application Publication
(10) Pub. No.: US 2021/0238310 A1
(43) Pub. Date: Aug. 5, 2021

(54) COMPOSITIONS AND METHODS RELATED TO ENGINEERED Fc-ANTIGEN BINDING DOMAIN CONSTRUCTS

(71) Applicant: Momenta Pharmaceuticals, Inc., Cambridge, MA (US)

(72) Inventors: Jonathan C. Lansing, Reading, MA (US); Daniel Ortiz, Stoneham, MA (US); Abhinav Gupta, Cambridge, MA (US)

(21) Appl. No.: 17/259,498

(22) PCT Filed: Jul. 11, 2019
(86) PCT No.: PCT/US2019/041492

§ 371 Date: Jan. 11, 2021

Related U.S. Application Data
(60) Provisional application No. 62/696,708, filed on Jul. 11, 2018.

(51) Int. Cl. C07K 16/42

(52) U.S. Cl. CPC C07K 16/42; C07K 2319/30; C07K 2317/524; C07K 2317/734; C07K 2317/53; C07K 2317/732; C07K 2317/526

(57) ABSTRACT

The present disclosure relates to compositions and methods of engineered Fc-antigen binding domain constructs, where the Fc-antigen binding domain constructs include at least two Fc domains and at least one antigen binding domain.

### CROSS-REFERENCE TO RELATED APPLICATIONS

This application is a National Stage application under 35 U.S.C. § 371 of International Application No. PCT/US2019/041492, having an International Filing Date of Jul. 11, 2019, which claims priority to U.S. Application Ser. No. 62/696,708, filed on Jul. 11, 2018. The disclosure of the prior application is considered part of the disclosure of this application, and is incorporated in its entirety into this application.

### SEQUENCE LISTING

The instant application contains a Sequence Listing which has been submitted electronically in ASCII format and is hereby incorporated by reference in its entirety. Said ASCII copy, created on Aug. 30, 2019, is named 14131-0183W01_SL.txt and is 251,435 bytes in size.

### BACKGROUND OF THE DISCLOSURE

Many therapeutic antibodies function by recruiting elements of the innate immune system through the effector function of the Fc domains, such as antibody-dependent cytotoxicity (ADCC), antibody-dependent cellular phagocytosis (ADCP), and complement-dependent cytotoxicity (CDC). There continues to be a need for improved therapeutic proteins.

### SUMMARY OF THE DISCLOSURE

The present disclosure features compositions and methods for combining the target-specificity of an antigen binding domain with at least two Fc domains to generate new therapeutics with unique biological activity. The compositions and methods described herein allow for the construction of proteins having multiple antigen binding domains and multiple Fc domains from multiple polypeptide chains. The number and spacing of antigen binding domains can be tuned to alter the binding properties (e.g., binding avidity) of the protein complexes for target antigens, and the number of Fc domains can be tuned to control the magnitude of effector functions to kill antigen-binding cells. Mutations (i.e., heterodimerizing and/or homodimerizing mutations, as described herein) are introduced into the polypeptides to reduce the number of undesired, alternatively assembled proteins that are produced. In some instances, heterodimerizing and/or homodimerizing mutations are introduced into the Fc domain monomers, and differentially mutated Fc domain monomers are placed among the different polypeptide chains that assemble into the protein, so as to control the assembly of the polypeptide chains into the desired protein structure. These mutations selectively stabilize the desired pairing of certain Fc domain monomers, and selectively destabilize the undesired pairings of other Fc domain monomers. In some cases, the Fc-antigen binding domain constructs are “orthogonal” Fc-antigen binding domain constructs that are formed by a first polypeptide containing multiple Fc domain monomers, in which at least two of the Fc monomers contain different heterodimerizing mutations (and thus differ from each other in sequence), e.g., a longer polypeptide with two or more Fc monomers with different heterodimerizing mutations, and at least two additional polypeptides that each contain at least one Fc monomer, wherein the Fc monomers of the additional polypeptides contain different heterodimerizing mutations from each other (and thus different sequences), e.g., two shorter polypeptides that each contain a single Fc domain monomer with different heterodimerizing mutations. The heterodimerizing mutations of the additional polypeptides are compatible with the heterodimerizing mutations of at least of Fc monomer of the first polypeptide.

In some instances, the present disclosure contemplates combining an antigen binding domain of a therapeutic protein with an Fc domain, e.g., a known therapeutic antibody, with at least two Fc domains to generate a novel therapeutic construct. To generate such constructs, the disclosure provides various methods for the assembly of constructs having at least two, e.g., multiple, Fc domains, and to control homodimerization and heterodimerization of such, to assemble molecules of discrete size from a limited number of polypeptide chains, which polypeptides are also a subject of the present disclosure. The properties of these constructs allow for the efficient generation of substantially homogenous pharmaceutical compositions. Such homogeneity in a pharmaceutical composition is desirable in order to ensure the safety, efficacy, uniformity, and reliability of the pharmaceutical composition. In some embodiments, the novel therapeutic constructs with at least two Fc domains described herein have a biological activity that is greater than that of a therapeutic protein with a single Fc domain.

In a first aspect, the disclosure features an Fc-antigen binding domain construct including at least one antigen binding domain and a first Fc domain joined to a second Fc domain by a linker. In some embodiments the Fc-antigen binding construct includes enhanced effector function, where the Fc-antigen binding domain construct includes at least one antigen binding domain and a first Fc domain joined to a second Fc domain by a linker, where the Fc-antigen binding domain construct has enhanced effector function in an antibody-dependent cytotoxicity (ADCC) assay, an antibody-dependent cellular phagocytosis (ADCP), and/or complement-dependent cytotoxicity (CDC) assay relative to a construct having a single Fc domain and the antigen binding domain.

In one aspect, the disclosure relates to a polypeptide comprising an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises mutations forming an engineered protuberance, and wherein at least one other Fc domain monomer comprises at least one, two or three reverse charge mutations.

In some embodiments, the antigen binding domain comprises an antibody heavy chain variable domain. In some embodiments, the antigen binding domain comprises an antibody light chain variable domain. In some embodiments, the first IgG1 Fc domain monomer comprises mutations forming an engineered protuberance and the second IgG1 Fc domain monomer comprises at least two reverse charge mutations. In some embodiments, the first IgG1 Fc domain monomer comprises at least two reverse charge mutations and the second IgG1 Fc domain monomer comprises mutations forming an engineered protuberance. In some embodiments, both the first IgG1 Fc domain monomer and the second IgG1 Fc domain monomer comprise mutations forming an engineered protuberance. In some embodiments, both the first IgG1 Fc domain monomer and the second IgG1 Fc domain monomer comprise at least two reverse charge mutations.

In some embodiments, the polypeptide comprises a third linker and a third IgG1 Fc domain monomer wherein the first IgG1 Fc domain monomer comprises mutations forming an engineered protuberance.

In some embodiments, the polypeptide comprises a third linker and a third IgG1 Fc domain monomer wherein the first IgG1 Fc domain monomer comprises at least two reverse charge mutations.

In some embodiments, the polypeptide comprises a third linker and a third IgG1 Fc domain monomer wherein the first IgG1 Fc domain monomer comprises mutations forming an engineered protuberance and both the second IgG1 Fc domain monomer and the third IgG1 Fc domain monomer each comprises at least two reverse charge mutations.

In some embodiments, the polypeptide comprises a third linker and third IgG1 Fc domain monomer wherein both the first IgG1 Fc domain monomer and the second IgG1 Fc domain monomer each comprise mutations forming an engineered protuberance and the third IgG1 domain monomer comprises at least two reverse charge mutations.

In some embodiments, IgG1 Fc domain monomers of the polypeptide that comprise mutations forming an engineered protuberance each have identical protuberance-forming mutations. In some embodiments, the IgG1 Fc domain monomers of the polypeptide that comprise reverse charge mutations each have identical reverse charge mutations.

In some embodiments, the IgG1 Fc domain monomers of the polypeptide comprising mutations forming an engineered protuberance further comprise at least one reverse charge mutation. In some embodiments, the IgG1 Fc domain monomers of the polypeptide comprising mutations forming an engineered protuberance and at least one reverse charge mutation comprise a reverse charge mutation that is different than the reverse charge mutation(s) of the IgG1 Fc domain monomers of the polypeptide that comprise reverse charge mutations but no protuberance-forming mutations.

In some embodiments, the mutations forming an engineered protuberance and the reverse charge mutations are in the CH3 domain. In some embodiments, the mutations are within the sequence from EU position G341 to EU position K447, inclusive. In some embodiments, the mutations are single amino acid changes.

In some embodiments, the second linker and the optional third linker comprise or consist of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 249), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22). In some embodiments, the second linker and the optional third linker is a glycine spacer. In some embodiments, the second linker and the optional third linker independently consist of 4 to 30 (SEQ ID NO: 250), 4 to 20 (SEQ ID NO: 251), 8 to 30 (SEQ ID NO: 252), 8 to 20 (SEQ ID NO: 253), 12 to 20 (SEQ ID NO: 254) or 12 to 30 (SEQ ID NO: 255) glycine residues. In some embodiments, the second linker and the optional third linker consist of 20 glycine residues (SEQ ID NO: 23).

In some embodiments, at least one of the Fc domain monomers comprises a single amino acid mutation at EU position I253. In some embodiments, each amino acid mutation at EU position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid mutation at position I253 is I253A.

In some embodiments, at least one of the Fc domain monomers comprises a single amino acid mutation at EU position R292. In some embodiments, each amino acid mutation at EU position R292 is independently selected from the group consisting of R292D, R292E, R292L, R292P, R292Q, R292R, R292T, and R292Y. In some embodiments, each amino acid mutation at position R292 is R292P.

In some embodiments, the hinge of each Fc domain monomer independently comprises or consists of an amino acid sequence selected from the group consisting of EPKSCDKTHTCPPCPAPELL (SEQ ID NO: 256) and DKTHTCPPCPAPELL (SEQ ID NO: 257). In some embodiments, the hinge portion of the second Fc domain monomer and the third Fc domain monomer have the amino acid sequence DKTHTCPPCPAPELL (SEQ ID NO: 257). In some embodiments, the hinge portion of the first Fc domain monomer has the amino acid sequence EPKSCDKTHTCPPCPAPEL (SEQ ID NO: 258). In some embodiments, the hinge portion of the first Fc domain monomer has the amino acid sequence EPKSCDKTHTCPPCPAPEL (SEQ ID NO: 258) and the hinge portion of the second Fc domain monomer and the third Fc domain monomer have the amino acid sequence DKTHTCPPCPAPELL (SEQ ID NO: 257).

In some embodiments, the CH2 domains of each Fc domain monomer independently comprise the amino acid sequence:

GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid deletions or substitutions. In some embodiments, the CH2 domains of each Fc domain monomer are identical and comprise the amino acid sequence:
GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid deletions or substitutions. In some embodiments, the CH2 domains of each Fc domain monomer are identical and comprise the amino acid sequence:
GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid substitutions. In some embodiments, the CH2 domains of each Fc domain monomer are identical and comprise the amino acid sequence:

### GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259).

In some embodiments, the CH3 domains of each Fc domain monomer independently comprise the amino acid sequence:

GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 10 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 8 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 6 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 5 single amino acid substitutions.

In some embodiments, the single amino acid substitutions are selected from the group consisting of: S354C, T366Y, T366W, T394W, T394Y, F405W, F405A, Y407A, S354C, Y349T, T394F, K409D, K409E, K392D, K392E, K370D, K370E, D399K, D399R, E357K, E357R, and D356K. In some embodiments, each of the Fc domain monomers independently comprises the amino acid sequence of any of SEQ ID NOs:42, 43, 45, and 47 having up to 10 single amino acid substitutions. In some embodiments, up to 6 of the single amino acid substitutions are reverse charge mutations in the CH3 domain or are mutations forming an engineered protuberance. In some embodiments, the single amino acid substitutions are within the sequence from Eu position G341 to Eu position K447, inclusive. In some embodiments, at least one of the mutations forming an engineered protuberance is selected from the group consisting of S354C, T366Y, T366W, T394W, T394Y, F405W, F405A, Y407A, S354C, Y349T, and T394F. In some embodiments, at least one reverse charge mutation is selected from: K409D, K409E, K392D. K392E, K370D, K370E, D399K, D399R, E357K, E357R, and D356K.

In some embodiments, the antigen binding domain is a scFv. In some embodiments, the antigen binding domain comprises a VH domain and a CH1 domain. In some embodiments, the antigen binding domain further comprises a VL domain. In some embodiments, the VH domain comprises a set of CDR-H1, CDR-H2 and CDR-H3 sequences set forth in Table 1A and 1B. In some embodiments, the VH domain comprises CDR-H1, CDR-H2, and CDR-H3 of a VH domain comprising a sequence of an antibody set forth in Table 2. In some embodiments, the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2. In some embodiments, the VH domain comprises a VH sequence of an antibody set forth in Table 2. In some embodiments, the antigen binding domain comprises a set of CDR-H1, CDR-H2, CDR-H3, CDR-L1, CDR-L2, and CDR-L3 sequences set forth in Table 1A and 1B. In some embodiments, the antigen binding domain comprises CDR-H1, CDR-H2, CDR-H3, CDR-L1, CDR-L2, and CDR-L3 sequences from a set of a VH and a VL sequence of an antibody set forth in Table 2. In some embodiments, the antigen binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2. In some embodiments, the antigen binding domain comprises a set of a VH and a VL sequence of an antibody set forth in Table 2. In some embodiments, the antigen binding domain comprises an IgG CL antibody constant domain and an IgG CH1 antibody constant domain. In some embodiments, the antigen binding domain comprises a VH domain and CH1 domain and can bind to a polypeptide comprising a VL domain and a CL domain to form a Fab.

In some embodiments, the disclosure relates to a polypeptide complex that comprises any of the foregoing polypeptides joined to a second polypeptide comprising an IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide. In some embodiments, the second polypeptide monomer comprises mutations forming an engineered cavity. In some embodiments, the mutations forming the engineered cavity are selected from the group consisting of: Y407T, Y407A, F405A, T394S, T394W/Y407A, T366W/T394S, T366S/L368A/Y407V/Y349C, S364H/F405A. In some embodiments, the second polypeptide monomer further comprises at least one reverse charge mutation. In some embodiments, the at least one reverse charge mutation is selected from: K409D, K409E, K392D. K392E, K370D, K370E, D399K, D399R, E357K, E357R, and D356K.

In some embodiments, the polypeptide complex is further joined to a third polypeptide comprising an IgG1 Fc domain monomer comprising a hinge domain, a CH2 domain and a CH3 domain, wherein the polypeptide and the third polypeptide are joined by disulfide bonds between cysteine residues within the hinge domain of the first, second or third IgG1 Fc domain monomer of the polypeptide and the hinge domain of the third polypeptide, wherein the second and third polypeptides join to different IgG1 Fc domain monomers of the polypeptide. In some embodiments, the third polypeptide monomer comprises at least two reverse charge mutations. In some embodiments, the at least two reverse charge mutations are selected from: K409D, K409E, K392D. K392E, K370D, K370E, D399K, D399R, E357K, E357R, and D356K.

In some embodiments, the second polypeptide monomer comprises at least one reverse charge mutation selected from the group consisting of K409D, K409E, K392D. K392E, K370D, K370E, D399K, D399R, E357K, E357R, and D356K and the third polypeptide monomer comprises at least two reverse charge mutations selected from the group consisting of K409D, K409E, K392D. K392E, K370D, K370E, D399K, D399R, E357K, E357R, and D356K, wherein the second and third polypeptide monomers comprise different reverse charge mutations.

In some embodiments, the second polypeptide comprises the amino acid sequence of any of SEQ ID NOs: 42, 43, 45, and 47 having up to 10 single amino acid substitutions. In some embodiments, the third polypeptide comprises the amino acid sequence of any of SEQ ID NOs: 42, 43, 45, and 47 having up to 10 single amino acid substitutions.

In some embodiments, the polypeptide comprises at least one Fc monomer comprising S354C and T366W mutations and at least one Fc monomer comprising D356K and D399K mutations. In some embodiments, the at least one Fc monomer comprising S354C and T366W mutations further comprises an E357K mutation. In some embodiments, the second polypeptide monomer comprises Y349C, T366S, L368A, and Y407V mutations. In some embodiments, the second polypeptide further comprises a K370D mutation. In some embodiments, the third polypeptide monomer comprises K392D and K409D mutations. In some embodiments, the second polypeptide monomer comprises Y349C, T366S, L368A, Y407V, and K370D mutations and the third polypeptide monomer comprises K392D and K409D mutations.

In some embodiments, the polypeptide complex comprises enhanced effector function in an antibody-dependent cytotoxicity (ADCC) assay, an antibody-dependent cellular phagocytosis (ADCP) and/or complement-dependent cytotoxicity (CDC) assay relative to a polypeptide complex having a single Fc domain and at least one antigen binding domain.

In another aspect, the disclosure relates to a polypeptide comprising a first IgG1 Fc domain monomer comprising a hinge domain, a CH2 domain and a CH3 domain; a first linker; a second IgG1 Fc domain monomer comprising a hinge domain, a CH2 domain and a CH3 domain; an optional second linker; and an optional third IgG1 Fc domain monomer comprising a hinge domain, a CH2 domain and a CH3 domain, wherein at least one Fc domain monomer comprises mutations forming an engineered protuberance, and wherein at least one other Fc domain monomer comprises at least one, two or three reverse charge mutations.

In some embodiments, the first IgG1 Fc domain monomer comprises mutations forming an engineered protuberance and the second IgG1 Fc domain monomer comprises at least two reverse charge mutations. In some embodiments, the first IgG1 Fc domain monomer comprises at least two reverse charge mutations and the second IgG1 Fc domain monomer comprises mutations forming an engineered protuberance. In some embodiments, both the first IgG1 Fc domain monomer and the second IgG1 Fc domain monomer comprise mutations forming an engineered protuberance. In some embodiments, both the first IgG1 Fc domain monomer and the second IgG1 Fc domain monomer comprise at least two reverse charge mutations.

In some embodiments, the polypeptide comprises a second linker and a third IgG1 Fc domain monomer wherein the first IgG1 Fc domain monomer comprises mutations forming an engineered protuberance.

In some embodiments, the polypeptide comprises a second linker and a third IgG1 Fc domain monomer wherein the first IgG1 Fc domain monomer comprises at least two reverse charge mutations.

In some embodiments, the polypeptide comprises a second linker and a third IgG1 Fc domain monomer wherein the first IgG1 Fc domain monomer comprises mutations forming an engineered protuberance and both the second IgG1 Fc domain monomer and the third IgG1 Fc domain monomer each comprises at least two reverse charge mutations.

In some embodiments, the polypeptide comprises a second linker and third IgG1 Fc domain monomer wherein both the first IgG1 Fc domain monomer and the second IgG1 Fc domain monomer each comprise mutations forming an engineered protuberance and the third IgG1 domain monomer comprises at least two reverse charge mutations.

In some embodiments, IgG1 Fc domain monomers of the polypeptide that comprise mutations forming an engineered protuberance each have identical protuberance-forming mutations. In some embodiments, the IgG1 Fc domain monomers of the polypeptide that comprise reverse charge mutations each have identical reverse charge mutations. In some embodiments, the IgG1 Fc domain monomers of the polypeptide comprising mutations forming an engineered protuberance further comprise at least one reverse charge mutation. In some embodiments, the IgG1 Fc domain monomers of the polypeptide comprising mutations forming an engineered protuberance and at least one reverse charge mutation comprise a reverse charge mutation that is different than the reverse charge mutation(s) of the IgG1 Fc domain monomers of the polypeptide that comprise reverse charge mutations but no protuberance-forming mutations.

In some embodiments, the mutations forming an engineered protuberance and the reverse charge mutations are in the CH3 domain. In some embodiments, the mutations are within the sequence from Eu position G341 to Eu position K447, inclusive. In some embodiments, the mutations are single amino acid changes.

In some embodiments, the first linker and the optional second linker comprise or consist of an amino acid sequence selected from the group consisting of:

GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 249), GENLYFQSGG (SEQ ID NO: 28), SACYCELS (SEQ ID NO: 29), RSIAT (SEQ ID NO: 30), RPACKIPNDLKQKVMNH (SEQ ID NO: 31),
GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22). In some embodiments, the first linker and the optional second linker is a glycine spacer. In some embodiments, the first linker and the optional second linker independently consist of 4 to 30 (SEQ ID NO: 250), 4 to 20 (SEQ ID NO: 251), 8 to 30 (SEQ ID NO: 252), 8 to 20 (SEQ ID NO: 253), 12 to 20 (SEQ ID NO: 254) or 12 to 30 (SEQ ID NO: 255) glycine residues. In some embodiments, the first linker and the optional second linker consist of 20 glycine residues (SEQ ID NO: 23).

In some embodiments, at least one of the Fc domain monomers comprises a single amino acid mutation at Eu position I253. In some embodiments, each amino acid mutation at Eu position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid mutation at position I253 is I253A.

In some embodiments, at least one of the Fc domain monomers comprises a single amino acid mutation at Eu position R292. In some embodiments, each amino acid mutation at Eu position R292 is independently selected from the group consisting of R292D, R292E, R292L, R292P, R292Q, R292R, R292T, and R292Y. In some embodiments, each amino acid mutation at position R292 is R292P.

In some embodiments, the hinge of each Fc domain monomer independently comprises or consists of an amino acid sequence selected from the group consisting of EPKSCDKTHTCPPCPAPELL

(SEQ ID NO: 256) and DKTHTCPPCPAPELL (SEQ ID NO: 257). In some embodiments, the hinge portion of the second Fc domain monomer and the third Fc domain monomer have the amino acid sequence DKTHTCPPCPAPELL (SEQ ID NO: 257). In some embodiments, the hinge portion of the first Fc domain monomer has the amino acid sequence DKTHTCPPCPAPELL (SEQ ID NO: 257). In some embodiments, the hinge portion of the first Fc domain monomer, the second Fc domain monomer and the third Fc domain monomer have the amino acid sequence DKTHTCPPCPAPELL (SEQ ID NO: 257).

In some embodiments, the CH2 domains of each Fc domain monomer independently comprise the amino acid sequence:

GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid deletions or substitutions. In some embodiments, the CH2 domains of each Fc domain monomer are identical and comprise the amino acid sequence:
GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid deletions or substitutions. In some embodiments, the CH2 domains of each Fc domain monomer are identical and comprise the amino acid sequence:
GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259) with no more than two single amino acid substitutions. In some embodiments, the CH2 domains of each Fc domain monomer are identical and comprise the amino acid sequence:

### GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK (SEQ ID NO: 259).

In some embodiments, the CH3 domains of each Fc domain monomer independently comprise the amino acid sequence:

GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 10 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 8 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 6 single amino acid substitutions. In some embodiments, the CH3 domains of each Fc domain monomer independently comprise the amino acid sequence:
GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 260) with no more than 5 single amino acid substitutions.

In some embodiments, the single amino acid substitutions are selected from the group consisting of: S354C, T366Y, T366W, T394W, T394Y, F405W, F405A, Y407A, S354C, Y349T, T394F, K409D, K409E, K392D, K392E, K370D, K370E, D399K, D399R, E357K, E357R, and D356K. In some embodiments, each of the Fc domain monomers independently comprises the amino acid sequence of any of SEQ ID NOs:42, 43, 45, and 47 having up to 10 single amino acid substitutions. In some embodiments, up to 6 of the single amino acid substitutions are reverse charge mutations in the CH3 domain or are mutations forming an engineered protuberance. In some embodiments, the single amino acid substitutions are within the sequence from Eu position G341 to Eu position K447, inclusive. In some embodiments, at least one of the mutations forming an engineered protuberance is selected from the group consisting of S354C, T366Y, T366W, T394W, T394Y, F405W, F405A, Y407A, S354C, Y349T, and T394F. In some embodiments, at least one reverse charge mutation is selected from: K409D, K409E, K392D. K392E, K370D, K370E, D399K, D399R, E357K, E357R, and D356K.

In some embodiments, the disclosure relates to a polypeptide complex comprising any of the foregoing polypeptides joined to a second polypeptide comprising an IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

In some embodiments, the second polypeptide monomer comprises mutations forming an engineered cavity. In some embodiments, the mutations forming the engineered cavity are selected from the group consisting of: Y407T, Y407A, F405A, T394S, T394W/Y407A, T366W/T394S, T366S/L368A/Y407V/Y349C, S364H/F405A. In some embodiments, the second polypeptide monomer further comprises at least one reverse charge mutation. In some embodiments, the at least one reverse charge mutation is selected from: K409D, K409E, K392D. K392E, K370D, K370E, D399K, D399R, E357K, E357R, and D356K.

In some embodiments, the polypeptide complex is further joined to a third polypeptide comprising an IgG1 Fc domain monomer comprising a hinge domain, a CH2 domain and a CH3 domain, wherein the polypeptide and the third polypeptide are joined by disulfide bonds between cysteine residues within the hinge domain of the first, second or third IgG1 Fc domain monomer of the polypeptide and the hinge domain of the third polypeptide, wherein the second and third polypeptides join to different IgG1 Fc domain monomers of the polypeptide.

In some embodiments, the third polypeptide monomer comprises at least two reverse charge mutations. In some embodiments, the at least two reverse charge mutations are selected from: K409D, K409E, K392D. K392E, K370D, K370E, D399K, D399R, E357K, E357R, and D356K.

In some embodiments, the second polypeptide monomer comprises at least one reverse charge mutation selected from the group consisting of K409D, K409E, K392D. K392E, K370D, K370E, D399K, D399R, E357K, E357R, and D356K and the third polypeptide monomer comprises at least two reverse charge mutations selected from the group consisting of K409D, K409E, K392D. K392E, K370D, K370E, D399K, D399R, E357K, E357R, and D356K, wherein the second and third polypeptide monomers comprise different reverse charge mutations.

In some embodiments, the second polypeptide comprises the amino acid sequence of any of SEQ ID NOs: 42, 43, 45, and 47 having up to 10 single amino acid substitutions. In some embodiments, the third polypeptide comprises the amino acid sequence of any of SEQ ID NOs: 42, 43, 45, and 47 having up to 10 single amino acid substitutions.

In some embodiments, the polypeptide comprises at least one Fc monomer comprising S354C and T366W mutations and at least one Fc monomer comprising D356K and D399K mutations. In some embodiments, the at least one Fc monomer comprising S354C and T366W mutations further comprises an E357K mutation. In some embodiments, the second polypeptide monomer comprises Y349C, T366S, L368A, and Y407V mutations. In some embodiments, the second polypeptide further comprises a K370D mutation. In some embodiments, the third polypeptide monomer comprises K392D and K409D mutations. In some embodiments, the second polypeptide monomer comprises Y349C, T366S, L368A, Y407V, and K370D mutations and the third polypeptide monomer comprises K392D and K409D mutations.

In some embodiments, the second polypeptide further comprises an antigen binding domain. In some embodiments, the third polypeptide further comprises an antigen binding domain. In some embodiments, the antigen binding domain comprises an antibody heavy chain variable domain. In some embodiments, the antigen binding domain comprises an antibody light chain variable domain. In some embodiments, the antigen binding domain is a scFv. In some embodiments, the antigen binding domain comprises a VH domain and a CH1 domain. In some embodiments, the antigen binding domain further comprises a VL domain. In some embodiments, the VH domain comprises a set of CDR-H1, CDR-H2 and CDR-H3 sequences set forth in Table 1A and 1B. In some embodiments, the VH domain comprises CDR-H1, CDR-H2, and CDR-H3 of a VH domain comprising a sequence of an antibody set forth in Table 2. In some embodiments, the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2. In some embodiments, the VH domain comprises a VH sequence of an antibody set forth in Table 2. In some embodiments, the antigen binding domain comprises a set of CDR-H1, CDR-H2, CDR-H3, CDR-L1, CDR-L2, and CDR-L3 sequences set forth in Table 1A and 1B. In some embodiments, the antigen binding domain comprises CDR-H1, CDR-H2, CDR-H3, CDR-L1, CDR-L2, and CDR-L3 sequences from a set of a VH and a VL sequence of an antibody set forth in Table 2. In some embodiments, the antigen binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2. In some embodiments, the antigen binding domain comprises a set of a VH and a VL sequence of an antibody set forth in Table 2. In some embodiments, the antigen binding domain comprises an IgG CL antibody constant domain and an IgG CH1 antibody constant domain. In some embodiments, the antigen binding domain comprises a VH domain and CH1 domain and can bind to a polypeptide comprising a VL domain and a CL domain to form a Fab. In some embodiments, the second polypeptide further comprises a first antigen binding domain and the third polypeptide further comprises an second antigen binding domain.

In some embodiments, the polypeptide complex comprises enhanced effector function in an antibody-dependent cytotoxicity (ADCC) assay, an antibody-dependent cellular phagocytosis (ADCP) and/or complement-dependent cytotoxicity (CDC) assay relative to a polypeptide complex having a single Fc domain and at least one antigen binding domain.

In another aspect, the disclosure relates to a nucleic acid molecule encoding the any of the foregoing polypeptides.

In another aspect, the disclosure relates to an expression vector comprising the nucleic acid molecule.

In another aspect, the disclosure relates to a host cell comprising the nucleic acid molecule.

In another aspect, the disclosure relates to a host cell comprising the expression vector.

In another aspect, the disclosure relates to a method of producing any of the foregoing polypeptides comprising culturing the host cell for a foregoing embodiments under conditions to express the polypeptide.

In some embodiments, the host cell further comprises a nucleic acid molecule encoding a polypeptide comprising an antibody VL domain. In some embodiments, the host cell further comprises a nucleic acid molecule encoding a polypeptide comprising an antibody VL domain. In some embodiments, the host cell further comprises a nucleic acid molecule encoding a polypeptide comprising an antibody VL domain and an antibody CL domain. In some embodiments, the host cell further comprises a nucleic acid molecule encoding a polypeptide comprising an antibody VL domain and an antibody CL domain. In some embodiments, the host cell further comprises a nucleic acid molecule encoding a polypeptide comprising an IgG1 Fc domain monomer having no more than 10 single amino acid mutations. In some embodiments, the host cell further comprises a nucleic acid molecule encoding a polypeptide comprising IgG1 Fc domain monomer having no more than 10 single amino acid mutations. In some embodiments, the IgG1 Fc domain monomer comprises the amino acid sequence of any of SEQ ID Nos; 42, 43, 45 and 47 having no more than 10, 8, 6 or 4 single amino acid mutations in the CH3 domain.

In another aspect, the disclosure relates to a pharmaceutical composition comprising any of the foregoing polypeptides.

In some embodiments, less than 40%, 30%, 20%, 10%, 5%, 2% of the polypeptides of the pharmaceutical composition have at least one fucose modification on an Fc domain monomer.

In another aspect, the disclosure relates to an Fc-antigen binding domain construct comprising: a) a first polypeptide comprising i) a first Fc domain monomer, ii) a second Fc domain monomer, iii) a third Fc domain monomer, iii) a linker joining the first Fc domain monomer and the second Fc domain monomer; and iv) a linker joining the second Fc domain monomer to the third Fc domain monomer; b) a second polypeptide comprising a fourth Fc domain monomer; c) a third polypeptide comprising a fifth Fc domain monomer; and d) an antigen binding domain joined to the first polypeptide and to the third polypeptide; wherein the first Fc domain monomer and the fourth Fc domain monomer combine to form a first Fc domain; wherein the second Fc domain monomer and the fourth Fc domain monomer combine to form a second Fc domain; and wherein the third Fc domain monomer and the fifth Fc domain monomer combine to form a third Fc domain.

In some embodiments, the linker comprises or consists of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 249), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22).

In some embodiments, the first and second Fc domain monomers comprise mutations forming an engineered protuberance and the third Fc domain monomer comprises at least two reverse charge mutations. In some embodiments, the first and second Fc domain monomers further comprise at least one reverse charge mutation.

In some embodiments, the mutations are single amino acid changes. In some embodiments, each of the Fc domain monomers independently comprises the amino acid sequence of any of SEQ ID NOs:42, 43, 45, and 47 having up to 10 single amino acid substitutions. In some embodiments, up to 6 of the single amino acid substitutions are reverse charge mutations in the CH3 domain or are mutations forming an engineered protuberance. In some embodiments, the single amino acid substitutions are within the sequence from Eu position G341 to EU position K447, inclusive.

In some embodiments, at least one of the mutations forming an engineered protuberance is selected from the group consisting of S354C, T366Y, T366W, T394W, T394Y, F405W, F405A, Y407A, S354C, Y349T, and T394F. In some embodiments, at least one reverse charge mutation is selected from: K409D, K409E, K392D. K392E, K370D, K370E, D399K, D399R, E357K, E357R, and D356K.

In some embodiments, the first and second Fc domain monomers each comprise S354C, T366W, and E357K mutations and the third Fc domain monomer comprises D356K and D399K mutations. In some embodiments, the fourth Fc domain monomer comprises Y349C, T366S, L368A, Y407V, and K370D mutations. In some embodiments, the fifth Fc domain monomer comprises K392D and K409D mutations.

In some embodiments, the antigen binding domain is a Fab. In some embodiments, the antigen binding domain is a scFv. In some embodiments, the antigen binding domain comprises a VH domain and a CH1 domain. In some embodiments, the antigen binding domain further comprises a VL domain. In some embodiments, the Fc-antigen binding domain construct comprises a fourth polypeptide comprising the VL domain. In some embodiments, the VH domain comprises a set of CDR-H1, CDR-H2 and CDR-H3 sequences set forth in Table 1A and 1B. In some embodiments, the VH domain comprises CDR-H1, CDR-H2, and CDR-H3 of a VH domain comprising a sequence of an antibody set forth in Table 2. In some embodiments, the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% identical to the VH sequence of an antibody set forth in Table 2. In some embodiments, the VH domain comprises a VH sequence of an antibody set forth in Table 2.

In another aspect, the disclosure relates to an Fc-antigen binding domain construct comprising: a) a first polypeptide comprising i) a first Fc domain monomer, ii) a second Fc domain monomer, iii) a third Fc domain monomer, iii) a linker joining the first Fc domain monomer and the second Fc domain monomer; and iv) a linker joining the second Fc domain monomer to the third Fc domain monomer; b) a second polypeptide comprising a fourth Fc domain monomer; c) a third polypeptide comprising a fifth Fc domain monomer; and d) an antigen binding domain joined to the first polypeptide and to the second polypeptide; wherein the first Fc domain monomer and the fourth Fc domain monomer combine to form a first Fc domain; wherein the second Fc domain monomer and the fourth Fc domain monomer combine to form a second Fc domain; and wherein the third Fc domain monomer and the fifth Fc domain monomer combine to form a third Fc domain.

In some embodiments, the linker comprises or consists of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 249), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22).

In some embodiments, the first and second Fc domain monomers each comprise mutations forming an engineered protuberance and the third Fc domain monomer comprises at least two reverse charge mutations. In some embodiments, the first and second Fc domain monomers further comprise at least one reverse charge mutation.

In some embodiments, the mutations are single amino acid changes. In some embodiments, each of the Fc domain monomers independently comprises the amino acid sequence of any of SEQ ID NOs:42, 43, 45, and 47 having up to 10 single amino acid substitutions. In some embodiments, up to 6 of the single amino acid substitutions are reverse charge mutations in the CH3 domain or are mutations forming an engineered protuberance. In some embodiments, the single amino acid substitutions are within the sequence from EU position G341 to EU position K447, inclusive.

In some embodiments, at least one of the mutations forming an engineered protuberance is selected from the group consisting of S354C, T366Y, T366W, T394W, T394Y, F405W, F405A, Y407A, S354C, Y349T, and T394F. In some embodiments, at least one reverse charge mutation is selected from: K409D, K409E, K392D. K392E, K370D, K370E, D399K, D399R, E357K, E357R, and D356K. In some embodiments, the first and second Fc domain monomers each comprise S354C, T366W, and E357K mutations and the third Fc domain monomer comprises D356K and D399K mutations. In some embodiments, the fourth Fc domain monomer comprises Y349C, T366S, L368A, Y407V, and K370D mutations. In some embodiments, the fifth Fc domain monomer comprises K392D and K409D mutations.

In some embodiments, the antigen binding domain is a Fab. In some embodiments, the antigen binding domain is a scFv. In some embodiments, the antigen binding domain comprises a VH domain and a CH1 domain. In some embodiments, the antigen binding domain further comprises a VL domain.

In some embodiments, the Fc-antigen binding domain construct comprises a fourth polypeptide comprising the VL domain. In some embodiments, the VH domain comprises a set of CDR-H1, CDR-H2 and CDR-H3 sequences set forth in Table 1A and 1B. In some embodiments, the VH domain comprises CDR-H1, CDR-H2, and CDR-H3 of a VH domain comprising a sequence of an antibody set forth in Table 2. In some embodiments, the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% identical to the VH sequence of an antibody set forth in Table 2. In some embodiments, the VH domain comprises a VH sequence of an antibody set forth in Table 2.

In another aspect, the disclosure relates to an Fc-antigen binding domain construct comprising: a) a first polypeptide comprising i) a first Fc domain monomer, ii) a second Fc domain monomer, iii) a third Fc domain monomer, iv) a linker joining the first Fc domain monomer and the second Fc domain monomer; and v) a linker joining the second Fc domain monomer to the third Fc domain monomer; b) a second polypeptide comprising a fourth Fc domain monomer; c) a third polypeptide comprising a fifth Fc domain monomer; and d) an antigen binding domain joined to the third polypeptide; wherein the first Fc domain monomer and the fourth Fc domain monomer combine to form a first Fc domain; wherein the second Fc domain monomer and the fifth Fc domain monomer combine to form a second Fc domain; and wherein the third Fc domain monomer and the fifth Fc domain monomer combine to form a third Fc domain.

In some embodiments, the linker comprises or consists of an amino acid sequence selected from the group consisting of: GGGGGGGGGGGGGGGGGGGG (SEQ ID NO: 23), GGGGS (SEQ ID NO: 1), GGSG (SEQ ID NO: 2), SGGG (SEQ ID NO: 3), GSGS (SEQ ID NO: 4), GSGSGS (SEQ ID NO: 5), GSGSGSGS (SEQ ID NO: 6), GSGSGSGSGS (SEQ ID NO: 7), GSGSGSGSGSGS (SEQ ID NO: 8), GGSGGS (SEQ ID NO: 9), GGSGGSGGS (SEQ ID NO: 10), GGSGGSGGSGGS (SEQ ID NO: 11), GGSG (SEQ ID NO: 2), GGSG (SEQ ID NO: 2), GGSGGGSG (SEQ ID NO: 12), GGSGGGSGGGSGGGGGSGGGGSGGGGSGGGGS (SEQ ID NO: 249), GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), GGSGGGSEGGGSEGGGSEGGGSEGGGSEGGGSGGGS (SEQ ID NO: 34), GGGSGGGSGGGS (SEQ ID NO: 35), SGGGSGGGSGGGSGGGSGGG (SEQ ID NO: 18), GGSGGGSGGGSGGGSGGS (SEQ ID NO: 36), GGGG (SEQ ID NO: 19), GGGGGGGG (SEQ ID NO: 20), GGGGGGGGGGGG (SEQ ID NO: 21) and GGGGGGGGGGGGGGGG (SEQ ID NO: 22).

In some embodiments, the first Fc domain monomer comprises mutations forming an engineered protuberance and the second and third Fc domain monomers each comprise at least two reverse charge mutations. In some embodiments, the first Fc domain monomer further comprises at least one reverse charge mutation.

In some embodiments, the mutations are single amino acid changes. In some embodiments, each of the Fc domain monomers independently comprises the amino acid sequence of any of SEQ ID NOs:42, 43, 45, and 47 having up to 10 single amino acid substitutions. In some embodiments, up to 6 of the single amino acid substitutions are reverse charge mutations in the CH3 domain or are mutations forming an engineered protuberance. In some embodiments, the single amino acid substitutions are within the sequence from EU position G341 to EU position K447, inclusive.

In some embodiments, at least one of the mutations forming an engineered protuberance is selected from the group consisting of S354C, T366Y, T366W, T394W, T394Y, F405W, F405A, Y407A, S354C, Y349T, and T394F. In some embodiments, at least one reverse charge mutation is selected from: K409D, K409E, K392D. K392E, K370D, K370E, D399K, D399R, E357K, E357R, and D356K. In some embodiments, the first Fc domain monomer comprises S354C, T366W, and E357K mutations and the second and third Fc domain monomers each comprise D356K and D399K mutations. In some embodiments, the fourth Fc domain monomer comprises Y349C, T366S, L368A, Y407V, and K370D mutations. In some embodiments, the fifth Fc domain monomer comprises K392D and K409D mutations.

In some embodiments, the antigen binding domain is a Fab. In some embodiments, the antigen binding domain is a scFv. In some embodiments, the antigen binding domain comprises a VH domain and a CH1 domain. In some embodiments, the antigen binding domain further comprises a VL domain. In some embodiments, the Fc-antigen binding domain construct comprises a fourth polypeptide comprising the VL domain. In some embodiments, the VH domain comprises a set of CDR-H1, CDR-H2 and CDR-H3 sequences set forth in Table 1A and 1B. In some embodiments, the VH domain comprises CDR-H1, CDR-H2, and CDR-H3 of a VH domain comprising a sequence of an antibody set forth in Table 2. In some embodiments, the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% identical to the VH sequence of an antibody set forth in Table 2. In some embodiments, the VH domain comprises a VH sequence of an antibody set forth in Table 2.

In another aspect, the disclosure relates to a method of manufacturing an Fc-antigen binding domain construct, the method comprising: a) culturing a host cell expressing: (1) a first polypeptide comprising i) a first Fc domain monomer, ii) a second Fc domain monomer, iii) a third Fc domain monomer, iv) a linker joining the first Fc domain monomer and the second Fc domain monomer; v) a linker joining the second Fc domain monomer to the third Fc domain monomer; (2) a second polypeptide comprising a fourth Fc domain monomer; (3) a third polypeptide comprising a fifth Fc domain monomer; and (4) an antigen binding domain; wherein the first Fc domain monomer and the fourth Fc domain monomer combine to form a first Fc domain, the second Fc domain monomer and the fourth Fc domain monomer combine to form a second Fc domain, and the third Fc domain monomer and the fifth Fc domain monomer combine to form a third Fc domain; wherein the antigen binding domain is joined to the first polypeptide and to the third polypeptide, thereby forming an Fc-antigen binding domain construct; and b) purifying the Fc-antigen binding domain construct from the cell culture supernatant.

In some embodiments, at least 50% of the Fc-antigen binding domain constructs in the cell culture supernatant, on a molar basis, are structurally identical.

In another aspect, the disclosure relates to a method of manufacturing an Fc-antigen binding domain construct, the method comprising: a) culturing a host cell expressing: (1) a first polypeptide comprising i) a first Fc domain monomer, ii) a second Fc domain monomer, iii) a third Fc domain monomer, iv) a linker joining the first Fc domain monomer and the second Fc domain monomer; v) a linker joining the second Fc domain monomer to the third Fc domain monomer; (2) a second polypeptide comprising a fourth Fc domain monomer; (3) a third polypeptide comprising a fifth Fc domain monomer; and (4) an antigen binding domain; wherein the first Fc domain monomer and the fourth Fc domain monomer combine to form a first Fc domain, the second Fc domain monomer and the fourth Fc domain monomer combine to form a second Fc domain, and the third Fc domain monomer and the fifth Fc domain monomer combine to form a third Fc domain; wherein the antigen binding domain is joined to the first polypeptide and to the second polypeptide, thereby forming an Fc-antigen binding domain construct; and b) purifying the Fc-antigen binding domain construct from the cell culture supernatant.

In some embodiments, at least 50% of the Fc-antigen binding domain constructs in the cell culture supernatant, on a molar basis, are structurally identical.

In another aspect, the disclosure relates to a method of manufacturing an Fc-antigen binding domain construct, the method comprising: a) culturing a host cell expressing: (1) a first polypeptide comprising i) a first Fc domain monomer, ii) a second Fc domain monomer, iii) a third Fc domain monomer, iv) a linker joining the first Fc domain monomer and the second Fc domain monomer; v) a linker joining the second Fc domain monomer to the third Fc domain monomer; (2) a second polypeptide comprising a fourth Fc domain monomer; (3) a third polypeptide comprising a fifth Fc domain monomer; and (4) an antigen binding domain; wherein the first Fc domain monomer and the fourth Fc domain monomer combine to form a first Fc domain, the second Fc domain monomer and the fifth Fc domain monomer combine to form a second Fc domain, and the third Fc domain monomer and the fifth Fc domain monomer combine to form a third Fc domain; wherein the antigen binding domain is joined to the third polypeptide, thereby forming an Fc-antigen binding domain construct; and b) purifying the Fc-antigen binding domain construct from the cell culture supernatant.

In some embodiments, at least 50% of the Fc-antigen binding domain constructs in the cell culture supernatant, on a molar basis, are structurally identical.

In all aspects of the disclosure, some or all of the Fc domain monomers (e.g., an Fc domain monomer comprising the amino acid sequence of any of SEQ ID Nos; 42, 43, 45 and 47 having no more than 10, 8, 6 or 4 single amino acid substitutions (e.g., in the CH3 domain only) can have one or both of a E345K and E43G amino acid substitution in addition to other amino acid substitutions or modifications. The E345K and E43G amino acid substitutions can increase Fc domain multimerization.

### Definitions

As used herein, the term “Fc domain monomer” refers to a polypeptide chain that includes at least a hinge domain and second and third antibody constant domains (CH2 and CH3) or functional fragments thereof (e.g., at least a hinge domain or functional fragment thereof, a CH2 domain or functional fragment thereof, and a CH3 domain or functional fragment thereof) (e.g., fragments that that capable of (i) dimerizing with another Fc domain monomer to form an Fc domain, and (ii) binding to an Fc receptor). A preferred Fc domain monomer comprises, from amino to carboxy terminus, at least a portion of IgG1 hinge, an IgG1 CH2 domain and an IgG1 CH3 domain. Thus, an Fc domain monomer, e.g., aa human IgG1 Fc domain monomer can extend from E316 to G446 or K447, from P317 to G446 or K447, from K318 to G446 or K447, from K318 to G446 or K447, from S319 to G446 or K447, from C320 to G446 or K447, from D321 to G446 or K447, from K322 to G446 or K447, from T323 to G446 or K447, from K323 to G446 or K447, from H324 to G446 or K447, from T325 to G446 or K447, or from C326 to G446 or K447. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., human IgG1). The human IgG1 Fc domain monomer is used in the examples described herein. The full hinge domain of human IgG1 extends from EU Numbering E316 to P230 or L235, the CH2 domain extends from A231 or G236 to K340 and the CH3 domain extends from G341 to K447. There are differing views of the position of the last amino acid of the hinge domain. It is either P230 or L235. In many examples herein the CH3 domain does not include K347. Thus, a CH3 domain can be from G341 to G446. In many examples herein a hinge domain can include E216 to L235. This is true, for example, when the hinge is carboxy terminal to a CH1 domain or a CD38 binding domain. In some case, for example when the hinge is at the amino terminus of a polypeptide, the Asp at EU Numbering 221 is mutated to Gln. An Fc domain monomer does not include any portion of an immunoglobulin that is capable of acting as an antigen-recognition region, e.g., a variable domain or a complementarity determining region (CDR). Fc domain monomers can contain as many as ten changes from a wild-type (e.g., human) Fc domain monomer sequence (e.g., 1-10, 1-8, 1-6, 1-4 amino acid substitutions, additions, or deletions) that alter the interaction between an Fc domain and an Fc receptor. Fc domain monomers can contain as many as ten changes (e.g., single amino acid changes) from a wild-type Fc domain monomer sequence (e.g., 1-10, 1-8, 1-6, 1-4 amino acid substitutions, additions, or deletions) that alter the interaction between Fc domain monomers. In certain embodiments, there are up to 10, 8, 6 or 5 single amino acid substitution on the CH3 domain compared to the human IgG1 CH3 domain sequence:

GQPREPQVYTLPPSRDELTKNQVSLTCLVKGFYPSDIAVEWESNGQPENNYKTTPPVLDSDGSFFLYSKLTVDKSRWQQGNVFSCSV MHEALHNHYTQKSLSLSPG (SEQ ID NO: 260). Examples of suitable changes are known in the art.

As used herein, the term “Fc domain” refers to a dimer of two Fc domain monomers that is capable of binding an Fc receptor. In the wild-type Fc domain, the two Fc domain monomers dimerize by the interaction between the two CH3 antibody constant domains, as well as one or more disulfide bonds that form between the hinge domains of the two dimerizing Fc domain monomers.

In the present disclosure, the term “Fc-antigen binding domain construct” refers to associated polypeptide chains forming at least two Fc domains as described herein and including at least one “antigen binding domain.” Fc-antigen binding domain constructs described herein can include Fc domain monomers that have the same or different sequences. For example, an Fc-antigen binding domain construct can have three Fc domains, two of which includes IgG1 or IgG1-derived Fc domain monomers, and a third which includes IgG2 or IgG2-derived Fc domain monomers. In another non-limiting example, an Fc-antigen binding domain construct can have three Fc domains, two of which include a “protuberance-into-cavity pair” (also known as a “knobs-into-holes pair”) and a third which does not include a “protuberance-into-cavity pair,”, e.g., the third Fc domain includes one or more electrostatic steering mutations rather than a protuberance-into-cavity pair, or the third Fc domain has a wild type sequence (i.e., includes no mutations). An Fc domain forms the minimum structure that binds to an Fc receptor, e.g., FcγRI, FcγRIIa, FcγRIIb, FcγRIIIa, FcγRIIIb, or FcγRIV. In some cases, the Fc-antigen binding domain constructs are “orthogonal” Fc-antigen binding domain constructs that are formed by joining a first polypeptide containing multiple Fc domain monomers, in which at least two of the Fc monomers contain different heterodimerizing mutations (i.e., the Fc monomers each have different protuberance-forming mutations or each have different electrostatic steering mutations, or one monomer has protuberance-forming mutations and one monomer has electrostatic steering mutations), to at least two additional polypeptides that each contain at least one Fc monomer, wherein the Fc monomers of the additional polypeptides contain different heterodimerizing mutations from each other (i.e., the Fc monomers of the additional polypeptides have different protuberance-forming mutations or have different electrostatic steering mutations, or one monomer has protuberance-forming mutations and one monomer has electrostatic steering mutations). The heterodimerizing mutations of the additional polypeptides associate compatibly with the heterodimerizing mutations of at least of Fc monomer of the first polypeptide.

As used herein, the term “antigen binding domain” refers to a peptide, a polypeptide, or a set of associated polypeptides that is capable of specifically binding a target molecule. In some embodiments, the “antigen binding domain” is the minimal sequence of an antibody that binds with specificity to the antigen bound by the antibody. Surface plasmon resonance (SPR) or various immunoassays known in the art, e.g., Western Blots or ELISAs, can be used to assess antibody specificity for an antigen. In some embodiments, the “antigen binding domain” includes a variable domain or a complementarity determining region (CDR) of an antibody, e.g., one or more CDRs of an antibody set forth in Table 1, one or more CDRs of an antibody set forth in Table 2, or the VH and/or VL domains of an antibody set forth in Table 2. In some embodiments, the CD38 binding domain can include a VH domain and a CH1 domain, optionally with a VL domain. In other embodiments, the antigen (e.g., CD38) binding domain is a Fab fragment of an antibody or a scFv. Thus, a CD38 binding domain can include a “CD38 heavy chain binding domain” that comprises or consists of a VH domain and a CH1 domain and a “CD38 light chain binding domain” that comprises or consists of a VL domain and a C_{L} domain. A CD38 binding domain may also be a synthetically engineered peptide that binds a target specifically such as a fibronectin-based binding protein (e.g., a fibronectin type III domain (FN3) monobody).

As used herein, the term “Complementarity Determining Regions” (CDRs) refers to the amino acid residues of an antibody variable domain the presence of which are necessary for antigen binding. Each variable domain typically has three CDR regions identified as CDR-L1, CDR-L2 and CDR-L3, and CDR-H1, CDR-H2, and CDR-H3). Each complementarity determining region may include amino acid residues from a “complementarity determining region” as defined by Kabat (i.e., about residues 24-34 (CDR-L1), 50-56 (CDR-L2), and 89-97 (CDR-L3) in the light chain variable domain and 31-35 (CDR-H1), 50-65 (CDR-H2), and 95-102 (CDR-H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (CDR-L1), 50-52 (CDR-L2), and 91-96 (CDR-L3) in the light chain variable domain and 26-32 (CDR-H1), 53-55 (CDR-H2), and 96-101 (CDR-H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs include amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR includes amino acids from both a CDR as defined by Kabat and those of a hypervariable loop, the FR residues will be adjusted accordingly.

An “Fv” fragment is an antibody fragment which contains a complete antigen recognition and binding site. This region consists of a dimer of one heavy and one light chain variable domain in tight association, which can be covalent in nature, for example, in a scFv. It is in this configuration that the three CDRs of each variable domain interact to define an antigen binding site on the surface of the VH-VL dimer.

The “Fab” fragment contains a variable and constant domain of the light chain and a variable domain and the first constant domain (CH1) of the heavy chain. F(a13′)₂ antibody fragments include a pair of Fab fragments which are generally covalently linked near their carboxy termini by hinge cysteines.

“Single-chain Fv” or “scFv” antibody fragments include the VH and VL domains of antibody in a single polypeptide chain. Generally, the scFv polypeptide further includes a polypeptide linker between the VH and VL domains, which enables the scFv to form the desired structure for antigen binding.

As used herein, the term “antibody constant domain” refers to a polypeptide that corresponds to a constant region domain of an antibody (e.g., a CL antibody constant domain, a CH1 antibody constant domain, a CH2 antibody constant domain, or a CH3 antibody constant domain).

As used herein, the term “promote” means to encourage and to favor, e.g., to favor the formation of an Fc domain from two Fc domain monomers which have higher binding affinity for each other than for other, distinct Fc domain monomers. As is described herein, two Fc domain monomers that combine to form an Fc domain can have compatible amino acid modifications (e.g., engineered protuberances and engineered cavities, and/or electrostatic steering mutations) at the interface of their respective CH3 antibody constant domains. The compatible amino acid modifications promote or favor the selective interaction of such Fc domain monomers with each other relative to with other Fc domain monomers which lack such amino acid modifications or with incompatible amino acid modifications. This occurs because, due to the amino acid modifications at the interface of the two interacting CH3 antibody constant domains, the Fc domain monomers to have a higher affinity toward each other than to other Fc domain monomers lacking amino acid modifications.

As used herein, the term “dimerization selectivity module” refers to a sequence of the Fc domain monomer that facilitates the favored pairing between two Fc domain monomers. “Complementary” dimerization selectivity modules are dimerization selectivity modules that promote or favor the selective interaction of two Fc domain monomers with each other. Complementary dimerization selectivity modules can have the same or different sequences. Exemplary complementary dimerization selectivity modules are described herein, and can include complementary mutations selected from the engineered protuberance-forming and cavity-forming mutations of Table 3 or the electrostatic steering mutations of Table 4.

As used herein, the term “engineered cavity” refers to the substitution of at least one of the original amino acid residues in the CH3 antibody constant domain with a different amino acid residue having a smaller side chain volume than the original amino acid residue, thus creating a three dimensional cavity in the CH3 antibody constant domain. The term “original amino acid residue” refers to a naturally occurring amino acid residue encoded by the genetic code of a wild-type CH3 antibody constant domain. An engineered cavity can be formed by, e.g., any one or more of the cavity-forming substitution mutations of Table 3.

As used herein, the term “engineered protuberance” refers to the substitution of at least one of the original amino acid residues in the CH3 antibody constant domain with a different amino acid residue having a larger side chain volume than the original amino acid residue, thus creating a three dimensional protuberance in the CH3 antibody constant domain. The term “original amino acid residues” refers to naturally occurring amino acid residues encoded by the genetic code of a wild-type CH3 antibody constant domain. An engineered protuberance can be formed by, e.g., any one or more of the protuberance-forming substitution mutations of Table 3.

As used herein, the term “protuberance-into-cavity pair” describes an Fc domain including two Fc domain monomers, wherein the first Fc domain monomer includes an engineered cavity in its CH3 antibody constant domain, while the second Fc domain monomer includes an engineered protuberance in its CH3 antibody constant domain. In a protuberance-into-cavity pair, the engineered protuberance in the CH3 antibody constant domain of the first Fc domain monomer is positioned such that it interacts with the engineered cavity of the CH3 antibody constant domain of the second Fc domain monomer without significantly perturbing the normal association of the dimer at the inter-CH3 antibody constant domain interface. A protuberance-into-cavity pair can include, e.g., a complementary pair of any one or more cavity-forming substitution mutation and any one or more protuberance-forming substitution mutation of Table 3.

As used herein, the term “heterodimer Fc domain” refers to an Fc domain that is formed by the heterodimerization of two Fc domain monomers, wherein the two Fc domain monomers contain different reverse charge mutations (see, e.g., mutations in Table 4) that promote the favorable formation of these two Fc domain monomers.

As used herein, the term “structurally identical,” in reference to a population of Fc-antigen binding domain constructs, refers to constructs that are assemblies of the same polypeptide sequences in the same ratio and configuration and does not refer to any post-translational modification, such as glycosylation.

As used herein, the term “homodimeric Fc domain” refers to an Fc domain that is formed by the homodimerization of two Fc domain monomers, wherein the two Fc domain monomers contain the same reverse charge mutations (see, e.g., mutations in Tables 5 and 6).

As used herein, the term “heterodimerizing selectivity module” refers to engineered protuberances, engineered cavities, and certain reverse charge amino acid substitutions that can be made in the CH3 antibody constant domains of Fc domain monomers in order to promote favorable heterodimerization of two Fc domain monomers that have compatible heterodimerizing selectivity modules. Fc domain monomers containing heterodimerizing selectivity modules may combine to form a heterodimeric Fc domain. Examples of heterodimerizing selectivity modules are shown in Tables 3 and 4.

As used herein, the term “homodimerizing selectivity module” refers to reverse charge mutations in an Fc domain monomer in at least two positions within the ring of charged residues at the interface between CH3 domains that promote homodimerization of the Fc domain monomer to form a homodimeric Fc domain. For example, the reverse charge mutations that form a homodimerizing selectivity module can be in at least two amino acids from positions 357, 370, 399, and/or 409 (by EU numbering), which are within the ring of charged residues at the interface between CH3 domains. Examples of homodimerizing selectivity modules are shown in Tables 4 and 5.

As used herein, the term “joined” is used to describe the combination or attachment of two or more elements, components, or protein domains, e.g., polypeptides, by means including chemical conjugation, recombinant means, and chemical bonds, e.g., peptide bonds, disulfide bonds and amide bonds. For example, two single polypeptides can be joined to form one contiguous protein structure through chemical conjugation, a chemical bond, a peptide linker, or any other means of covalent linkage. In some embodiments, an antigen binding domain is joined to a Fc domain monomer by being expressed from a contiguous nucleic acid sequence encoding both the antigen binding domain and the Fc domain monomer. In other embodiments, an antigen binding domain is joined to a Fc domain monomer by way of a peptide linker, wherein the N-terminus of the peptide linker is joined to the C-terminus of the antigen binding domain through a chemical bond, e.g., a peptide bond, and the C-terminus of the peptide linker is joined to the N-terminus of the Fc domain monomer through a chemical bond, e.g., a peptide bond.

As used herein, the term “associated” is used to describe the interaction, e.g., hydrogen bonding, hydrophobic interaction, or ionic interaction, between polypeptides (or sequences within one single polypeptide) such that the polypeptides (or sequences within one single polypeptide) are positioned to form an Fc-antigen binding domain construct described herein (e.g., an Fc-antigen binding domain construct having three Fc domains). For example, in some embodiments, four polypeptides, e.g., two polypeptides each including two Fc domain monomers and two polypeptides each including one Fc domain monomer, associate to form an Fc construct that has three Fc domains (e.g., as depicted in FIGS. 50 and 51). The four polypeptides can associate through their respective Fc domain monomers. The association between polypeptides does not include covalent interactions.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer is part of the primary sequence of a polypeptide (e.g., joined to the spaced polypeptides or polypeptide domains via the polypeptide backbone). The formation of disulfide bonds, e.g., between two hinge regions or two Fc domain monomers that form an Fc domain, is not considered a linker.

As used herein, the term “glycine spacer” refers to a linker containing only glycines that joins two Fc domain monomers in tandem series. A glycine spacer may contain at least 4 (SEQ ID NO: 19), 8 (SEQ ID NO: 20), or 12 (SEQ ID NO: 21) glycines (e.g., 4-30 (SEQ ID NO: 250), 8-30 (SEQ ID NO: 252), or 12-30 (SEQ ID NO: 255) glycines; e.g., 12-30 (SEQ ID NO: 255), 4, 5, 6, 7, 8, 9, 10, 11, 12, 13, 14, 15, 16, 17, 18, 19, 20, 21, 22, 23, 24, 25, 26, 27, 28, 29, or 30 glycines (SEQ ID NO: 250)). In some embodiments, a glycine spacer has the sequence of GGGGGGGGGGGGGGGGGGGG (SEQ ID NO: 27). As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments of the present disclosure, an albumin-binding peptide is fused to the C-terminus of an Fc domain monomer to increase the serum half-life of the Fc-antigen binding domain construct. An albumin-binding peptide can be fused, either directly or through a linker, to the N- or C-terminus of an Fc domain monomer.

As used herein, the term “purification peptide” refers to a peptide of any length that can be used for purification, isolation, or identification of a polypeptide. A purification peptide may be joined to a polypeptide to aid in purifying the polypeptide and/or isolating the polypeptide from, e.g., a cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct are described in detail further herein.

As used herein, the term “multimer” refers to a molecule including at least two associated Fc constructs or Fc-antigen binding domain constructs described herein.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the term “polypeptide” describes a single polymer in which the monomers are amino acid residues which are joined together through amide bonds. A polypeptide is intended to encompass any amino acid sequence, either naturally occurring, recombinant, or synthetically produced.

As used herein, the term “amino acid positions” refers to the position numbers of amino acids in a protein or protein domain. The amino acid positions are numbered using the Kabat numbering system (Kabat et al., Sequences of Proteins of Immunological Interest, National Institutes of Health, Bethesda, Md., ed 5, 1991) where indicated (eg.g., for CDR and FR regions), otherwise the EU numbering is used.

FIGS. 17A-17D depict human IgG1 Fc domains numbered using the EU numbering system.

As used herein, the term “amino acid modification” or refers to an alteration of an Fc domain polypeptide sequence that, compared with a reference sequence (e.g., a wild-type, unmutated, or unmodified Fc sequence) may have an effect on the pharmacokinetics (PK) and/or pharmacodynamics (PD) properties, serum half-life, effector functions (e.g., cell lysis (e.g., antibody-dependent cell-mediated toxicity (ADCC) and/or complement dependent cytotoxicity activity (CDC)), phagocytosis (e.g., antibody dependent cellular phagocytosis (ADCP) and/or complement-dependent cellular cytotoxicity (CDCC)), immune activation, and T-cell activation), affinity for Fc receptors (e.g., Fc-gamma receptors (FcγR) (e.g., FcγRI (CD64), FcγRIIa (CD32), FcγRIIb (CD32), FcγRIIIa (CD16a), and/or FcγRIIIb (CD16b)), Fc-alpha receptors (FcaR), Fc-epsilon receptors (FcER), and/or to the neonatal Fc receptor (FcRn)), affinity for proteins involved in the compliment cascade (e.g., Clq), post-translational modifications (e.g., glycosylation, sialylation), aggregation properties (e.g., the ability to form dimers (e.g., homo- and/or heterodimers) and/or multimers), and the biophysical properties (e.g., alters the interaction between CH1 and C_{L}, alters stability, and/or alters sensitivity to temperature and/or pH) of an Fc construct, and may promote improved efficacy of treatment of immunological and inflammatory diseases. An amino acid modification includes amino acid substitutions, deletions, and/or insertions. In some embodiments, an amino acid modification is the modification of a single amino acid. In other embodiment, the amino acid modification is the modification of multiple (e.g., more than one) amino acids. The amino acid modification may include a combination of amino acid substitutions, deletions, and/or insertions. Included in the description of amino acid modifications, are genetic (i.e., DNA and RNA) alterations such as point mutations (e.g., the exchange of a single nucleotide for another), insertions and deletions (e.g., the addition and/or removal of one or more nucleotides) of the nucleotide sequence that codes for an Fc polypeptide.

In certain embodiments, at least one (e.g., one, two, or three) Fc domain within an Fc construct or Fc-antigen binding domain construct includes an amino acid modification. In some instances, the at least one Fc domain includes one or more (e.g., two, three, four, five, six, seven, eight, nine, ten, or twenty or more) amino acid modifications.

In certain embodiments, at least one (e.g., one, two, or three) Fc domain monomers within an Fc construct or Fc-antigen binding domain construct include an amino acid modification (e.g., substitution). In some instances, the at least one Fc domain monomers includes one or more (e.g., no more than two, three, four, five, six, seven, eight, nine, ten, or twenty) amino acid modifications (e.g., substitutions).

As used herein, the term “percent (%) identity” refers to the percentage of amino acid (or nucleic acid) residues of a candidate sequence, e.g., the sequence of an Fc domain monomer in an Fc-antigen binding domain construct described herein, that are identical to the amino acid (or nucleic acid) residues of a reference sequence, e.g., the sequence of a wild-type Fc domain monomer, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of a wild-type Fc domain monomer (e.g., SEQ ID NO: 42). In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of any one of SEQ ID NOs: 43-48, and 50-53. In certain embodiments, an Fc domain monomer in the Fc construct may have a sequence that is at least 95% identical (at least 97%, 99%, or 99.5% identical) to the sequence of SEQ ID NO: 48, 52, and 53.

In some embodiments, a spacer between two Fc domain monomers may have a sequence that is at least 75% identical (at least 75%, 77%, 79%, 81%, 83%, 85%, 87%, 89%, 91%, 93%, 95%, 97%, 99%, 99.5%, or 100% identical) to the sequence of any one of SEQ ID NOs: 1-36 (e.g., SEQ ID NOs: 17, 18, 26, and 27) described further herein.

In some embodiments, an Fc domain monomer in the Fc construct may have a sequence that differs from the sequence of any one of SEQ ID NOs: 42-48 and 50-53 by up to 10 amino acids, e.g., 1, 2, 3, 4, 5, 6, 7, 8, 9, or 10 amino acids. In some embodiments, an Fc domain monomer in the Fc construct has up to 10 amino acid substitutions relative to the sequence of any one of SEQ ID NOs: 42-48 and 50-53, e.g., 1, 2, 3, 4, 5, 6, 7, 8, 9, or 10 amino acid substitutions.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell). As described herein, a host cell is used to express one or more polypeptides encoding desired domains which can then combine to form a desired Fc-antigen binding domain construct.

As used herein, the term “pharmaceutical composition” refers to a medicinal or pharmaceutical formulation that contains an active ingredient as well as one or more excipients and diluents to enable the active ingredient to be suitable for the method of administration. The pharmaceutical composition of the present disclosure includes pharmaceutically acceptable components that are compatible with the Fc-antigen binding domain construct. The pharmaceutical composition is typically in aqueous form for intravenous or subcutaneous administration.

As used herein, a “substantially homogenous population” of polypeptides or of an Fc construct is one in which at least 50% of the polypeptides or Fc constructs in a composition (e.g., a cell culture medium or a pharmaceutical composition) have the same number of Fc domains, as determined by non-reducing SDS gel electrophoresis or size exclusion chromatography. A substantially homogenous population of polypeptides or of an Fc construct may be obtained prior to purification, or after Protein A or Protein G purification, or after any Fab or Fc-specific affinity chromatography only. In various embodiments, at least 55%, 60%, 65%, 70%, 75%, 80%, or 85% of the polypeptides or Fc constructs in the composition have the same number of Fc domains. In other embodiments, up to 85%, 90%, 92%, or 95% of the polypeptides or Fc constructs in the composition have the same number of Fc domains.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc-antigen binding domain construct. The nature of the carrier differs with the mode of administration. For example, for oral administration, a solid carrier is preferred; for intravenous administration, an aqueous solution carrier (e.g., WFI, and/or a buffered solution) is generally used.

As used herein, “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term fragment and the term portion can be used interchangeably.

### BRIEF DESCRIPTION OF THE DRAWINGS

FIG. 1 is a schematic showing a tandem construct with two Fc domains (formed by joining identical polypeptide chains together) and some of the resulting species generated by off-register association of the tandem Fc sequences. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, and the hinge disulfides are shown as pairs of parallel lines.

FIG. 2 is a schematic showing a tandem construct with three Fc domains connected by peptide linkers (formed by joining identical polypeptide chains together) and some of the resulting species generated by off-register association of the tandem Fc sequences. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, and the hinge disulfides are shown as pairs of parallel lines.

FIGS. 3A and 3B are schematics of Fc constructs with two Fc domains (FIG. 3A) or three Fc domains (FIG. 3B) connected by linkers and assembled using orthogonal heterodimerization domains. Each of the unique polypeptide chains is shaded differently. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, the linkers are shown as dashed lines, and the hinge disulfides are shown as pairs of parallel lines. CH3 ovals are shown with protuberances to depict knobs and cavities to depict holes for knob-into-holes pairs. Plus and/or minus signs are used to depict electrostatic steering mutations in the CH3 domain.

FIGS. 4A-H are schematics of Fc constructs with multiple Fc domains in tandem that are assembled using orthogonal heterodimerization domains. Each of the unique polypeptide chains is shaded differently. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, the linkers are shown as dashed lines, and the hinge disulfides are shown as pairs of parallel lines. The Fc domains utilizing a first set of heterodimerization mutations in the Fc monomers of the domains are denoted A and B. The Fc domains utilizing a second set of heterodimerization mutations in the Fc monomers of the domains are denoted C and D.

FIGS. 5A-F are schematics of branched Fc constructs with multiple symmetrically-distributed Fc domains that are assembled by an asymmetrical arrangement of polypeptide chains using orthogonal heterodimerization domains. Each of the unique polypeptide chains is shaded differently. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, the linkers are shown as dashed lines, and the hinge disulfides are shown as pairs of parallel lines. The Fc domains utilizing a first set of heterodimerization mutations in the Fc monomers of the domains are denoted A and B. The Fc domains utilizing a second set of heterodimerization mutations in the Fc monomers of the domains are denoted C and D.

FIGS. 6A-F are schematics of branched Fc constructs with multiple asymmetrically-distributed Fc domains that are assembled by an asymmetrical arrangement of polypeptide chains using orthogonal heterodimerization domains. Each of the unique polypeptide chains is shaded differently. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, the linkers are shown as dashed lines, and the hinge disulfides are shown as pairs of parallel lines. The Fc domains utilizing a first set of heterodimerization mutations in the Fc monomers of the domains are denoted A and B. The Fc domains utilizing a second set of heterodimerization mutations in the Fc monomers of the domains are denoted C and D.

FIGS. 7A-D are schematics of branched Fc constructs with symmetrically-distributed Fc domains and asymmetrically distributed Fab(s) that are assembled by an asymmetrical arrangement of polypeptide chains using orthogonal heterodimerization domains. Each of the unique polypeptide chains is shaded differently. The variable domains of the Fab portion (VH+VL) are depicted as parallelograms, the constant domains of the Fab portion (CH1+CL) are depicted as rectangles, the domains of the Fc portion (CH2 and CH3) are depicted as ovals, the linkers are shown as dashed lines, and the hinge disulfides are shown as pairs of parallel lines. The Fc domains utilizing a first set of heterodimerization mutations in the Fc monomers of the domains are denoted A and B. The Fc domains utilizing a second set of heterodimerization mutations in the Fc monomers of the domains are denoted C and D.

FIG. 8 is a schematic of a branched anti-CD20 construct with a single asymmetrically-distributed Fab used to demonstrate the expression of asymmetrically branched Fc constructs.

FIG. 9 is a schematic of a branched anti-CD20 construct with a single asymmetrically-distributed Fab used to demonstrate the expression of asymmetrically branched Fc constructs.

FIG. 10 shows the results of an SDS-PAGE analysis of cells transfected with genes encoding the polypeptides that assemble into the Fc construct of FIG. 8. The presence of a 200 kDa band in the leftmost lane (lane 1) demonstrates the formation of the intended Fc construct.

FIG. 11 shows the results of an SDS-PAGE analysis of cells transfected with genes encoding the polypeptides that assemble into the Fc construct of FIG. 9. The presence of a band in the leftmost lane (lane 1) with a molecular weight that is slightly higher than 200 kDa demonstrates the formation of the intended Fc construct.

FIG. 12 is an illustration of an Fc-antigen binding domain construct (construct 45) containing three Fc domains and two antigen binding domains. The construct is formed of four Fc domain monomer containing polypeptides. The first polypeptide (4502) contains one Fc domain monomer with a first set of CH3 charged amino acid substitutions (4510) and two Fc domain monomers, each with the same protuberance-forming amino acid substitutions optionally with a second set of CH3 charged amino acid substitution(s) (4508 and 4506), linked by spacers in a tandem series to an antigen binding domain containing a VH domain (4512) at the N-terminus. The second polypeptide (4524) contains one Fc domain monomer with a set of charged amino acid substitution(s) (4522) that promote favorable electrostatic interaction with the Fc domain monomer of the first polypeptide with the first set of charged amino acid substitutions (4510), joined in a tandem series to an antigen binding domain containing a VH domain (4518) at the N-terminus. The third and fourth polypeptides (4516 and 4514) each contain one Fc domain monomer with cavity-forming amino acid substitutions optionally with a set of CH3 charged amino acid substitution(s) that promote favorable electrostatic interaction with the Fc domai monomers of the first polypeptide with a second set of charged amino acid substitutions (4508 and 4506). A VL containing domain (4504, and 4520) is joined to each VH domain.

FIG. 13 is an illustration of an Fc-antigen binding domain construct (construct 46) containing three Fc domains and two antigen binding domains. The construct is formed of four Fc domain monomer containing polypeptides. The first polypeptide (4602) contains one Fc domain monomer with a first set of CH3 charged amino acid substitutions (4608) and two Fc domain monomers, each with the same protuberance-forming amino acid substitutions optionally with a second set of CH3 charged amino acid substitution(s) (4606 and 4604), linked by spacers in a tandem series. The second polypeptide (4618) contains one Fc domain monomer with a set of charged amino acid substitution(s) that promote favorable electrostatic interaction with the Fc domain monomer of the first polypeptide with the first set of charged amino acid substitutions (4608). The third and fourth polypeptides (4626 and 4624) each contain one Fc domain monomer with cavity-forming amino acid substitutions optionally with a set of CH3 charged amino acid substitution(s) that promote favorable electrostatic interaction with the Fc domain monomers of the first polypeptide with a second set of charged amino acid substitutions (4606 and 4604), joined in a tandem series to an antigen binding domain containing a V_{H} domain (4622 and 4620) at the N-terminus. A V_{L} containing domain (4614 and 4610) is joined to each V_{H} domain.

FIG. 14 is an illustration of an Fc-antigen binding domain construct (construct 47) containing three Fc domains and two antigen binding domains. The construct is formed of four Fc domain monomer containing polypeptides. The first polypeptide (4702) contains two Fc domain monomers, each with a first set of C_{H}3 charged amino acid substitutions (4708 and 4706) and one Fc domain monomer with protuberance-forming amino acid substitutions optionally with a second set of C_{H}3 charged amino acid substitution(s) (4704), linked by spacers in a tandem series. The second and third polypeptides (4726 and 4724) each contain one Fc domain monomer with a set of charged amino acid substitution(s) that promote favorable electrostatic interaction with the Fc domain monomers of the first polypeptide with the first set of charged amino acid substitutions (4708 and 4706), joined in a tandem series to an antigen binding domain containing a V_{H} domain (4722 and 4720) at the N-terminus. The fourth polypeptide (4710) contains one Fc domain monomer with cavity-forming amino acid substitutions optionally with a set of C_{H}3 charged amino acid substitution(s) that promote favorable electrostatic interaction with the Fc domain monomer of the first polypeptide with a second set of charged amino acid substitutions (4704). A V_{L} containing domain (4712 and 4716) is joined to each V_{H} domain.

FIG. 15 is an illustration of an Fc-antigen binding domain construct (construct 48) containing five Fc domains and four antigen binding domains. The construct is formed from six Fc domain monomer containing polypeptides. The first polypeptide (4802) contains four Fc domain monomers, each with the same protuberance-forming amino acid substitutions optionally with a first set of C_{H}3 charged amino acid substitution(s) (4812, 4810, 4808, and 4806) and one Fc domain monomer with a second set of C_{H}3 charged amino acid substitutions (4804), linked by spacers in a tandem series. The second, third, fourth, and fifth polypeptides (4846, 4844, 4842, and 4840) each contain one Fc domain monomer with cavity-forming amino acid substitutions optionally with a set of C_{H}3 charged amino acid substitution(s) (4830, 4826, 4822, and 4818) that promote favorable electrostatic interaction with the Fc domain monomers of the first polypeptide with a first set of charged amino acid substitutions (4812, 4810, 4808, and 4806), joined in a tandem series to an antigen binding domain containing a V_{H} domain (4838, 4836, 4834, and 4832) at the N-terminus. The sixth polypeptide (4814) contains one Fc domain monomer with a set of charged amino acid substitution(s) that promote favorable electrostatic interaction with the Fc domain monomer of the first polypeptide with the second set of charged amino acid substitutions (4804). A V_{L} containing domain (4816, 4820, 4824, and 4828) is joined to each V_{H} domain.

FIG. 16A-C is a schematic representation of three exemplary ways the antigen binding domain can be joined to the Fc domain of an Fc construct. FIG. 16A shows a heavy chain component of an antigen binding domain can be expressed as a fusion protein of an Fc chain and a light chain component can be expressed as a separate polypeptide. FIG. 16B shows an scFv expressed as a fusion protein of the long Fc chain. FIG. 16C shows heavy chain and light chain components expressed separately and exogenously added and joined to the Fc-antigen binding domain construct with a chemical bond.

FIG. 17A depicts the amino acid sequence of a human IgG1 (SEQ ID NO: 43) with EU numbering. The hinge region is indicated by a double underline, the CH2 domain is not underlined and the CH3 region is underlined.

FIG. 17B depicts the amino acid sequence of a human IgG1 (SEQ ID NO: 45) with EU numbering. The hinge region, which lacks E216-C220, inclusive, is indicated by a double underline, the CH2 domain is not underlined and the CH3 region is underlined and lacks K447.

FIG. 17C depicts the amino acid sequence of a human IgG1 (SEQ ID NO: 47) with EU numbering. The hinge region is indicated by a double underline, the CH2 domain is not underlined and the CH3 region is underlined and lacks 447K.

FIG. 17D depicts the amino acid sequence of a human IgG1 (SEQ ID NO: 42) with EU numbering. The hinge region, which lacks E216-C220, inclusive, is indicated by a double underline, the CH2 domain is not underlined and the CH3 region is underlined.

FIG. 18 is a schematic of a branched alternative anti-CD20 construct with a single asymmetrically-distributed Fab used to demonstrate the expression of asymmetrically branched Fc constructs.

FIG. 19 is a schematic of a branched alternative anti-CD20 construct with a single asymmetrically-distributed Fab used to demonstrate the expression of asymmetrically branched Fc constructs.

FIG. 20 depicts the amino acid sequences (SEQ ID NOS 325-326, 236, and 61, respectively, in order of appearance) of polypeptides that can be used to create a branched alternative anti-CD20 construct with a single asymmetrically-distributed Fab such as that depicted in FIG. 18.

FIG. 21 depicts the amino acid sequences (SEQ ID NOS 325, 327, 48, and 61, respectively, in order of appearance) of polypeptides that can be used to create a branched alternative anti-CD20 construct with a single asymmetrically-distributed Fab such as that depicted in FIG. 18.

### DETAILED DESCRIPTION

Many therapeutic antibodies function by recruiting elements of the innate immune system through the effector function of the Fc domains, such as antibody-dependent cytotoxicity (ADCC), antibody-dependent cellular phagocytosis (ADCP), and complement-dependent cytotoxicity (CDC). In some instances, the present disclosure contemplates combining an antigen binding domain with at least two Fc domains to generate a novel therapeutic. In some cases, the present disclosure contemplates combining an antigen binding domain of a single Fc-domain containing therapeutic, e.g., a known therapeutic antibody, with at least two Fc domains to generate a novel therapeutic with unique biological activity. In some instances, a novel therapeutic disclosed herein has a biological activity greater than that of the single Fc-domain containing therapeutic, e.g., a known therapeutic antibody. The presence of at least two Fc domains can enhance effector functions and to activate multiple effector functions, such as ADCC in combination with ADCP and/or CDC, thereby increasing the efficacy of the therapeutic molecules.

The methods and compositions described herein allow for the construction of antigen-binding proteins with multiple Fc domains by introducing multiple orthogonal heterodimerization technologies (e.g., two different sets of mutations selected from Tables 3 and 4) optionally with homodimerizing technologies (e.g., mutations selected from Tables 5 and 6) into the polypeptides that join together to form the same protein. The design principles described herein, which introduce multiple heterodimerizing mutations into the polypeptides that assemble into the same protein, allow for the creation of a great diversity of protein configurations, including, e.g., antibody-like proteins with tandem Fc domains, symmetrically branched proteins, and asymmetrically branched proteins. The design principles described herein allow for the controlled creation of complex protein configurations while disfavoring the formation of undesired higher-order structures or of uncontrolled complexes. The orthogonal Fc-antigen binding domain constructs described herein contain at least one antigen-binding domain and at least two Fc domains that are joined together by a linker, wherein at least two of the Fc domains differ from each other, e.g., at least one Fc domain of the construct is joined to an antigen-binding domain and at least one Fc domain of the construct is not joined to an antigen-binding domain, or two Fc domains of the construct are joined to different antigen-binding domains. The orthogonal Fc-antigen binding domain constructs are manufactured by expressing one long peptide chain containing two or more Fc monomers separated by linkers and expressing two or more different short peptide chains that each contain a single Fc monomer that is designed to bind preferentially to one or more particular Fc monomers on the long peptide chain. Any number of Fc domains can be connected in tandem in this fashion, allowing the creation of constructs with 2, 3, 4, 5, 6, 7, 8, 9, 10, or more Fc domains.

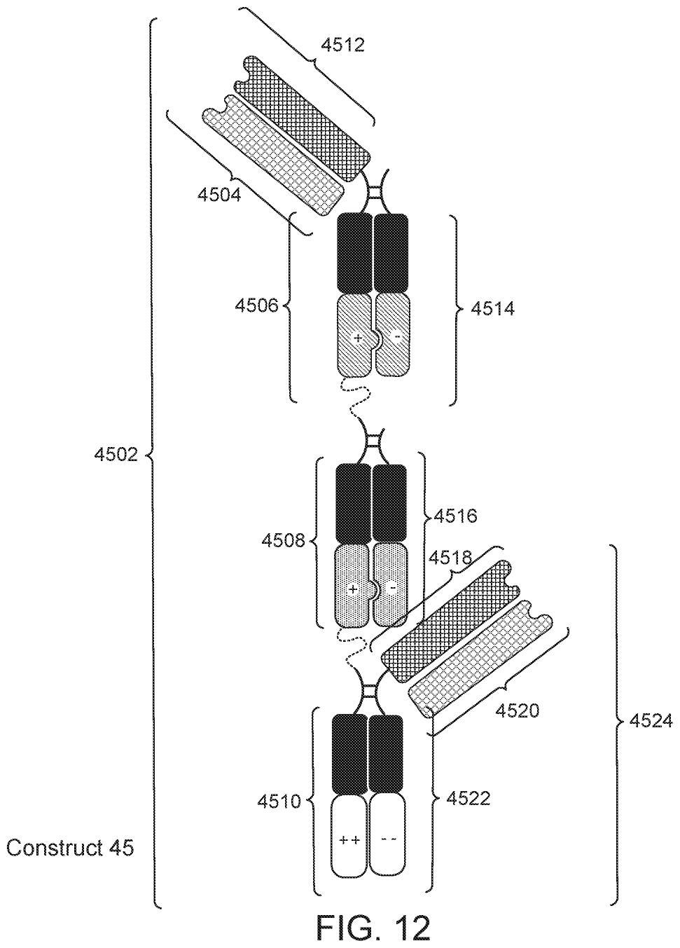

The orthogonal Fc-antigen binding domain constructs are created using the Fc engineering methods for assembling molecules with two or more Fc domains described in PCT/US2018/012689 and in International Publication Nos. WO/2015/168643, WO2017/151971, WO 2017/205436, and WO 2017/205434, which are herein incorporated by reference in their entirety. The engineering methods make use of two sets of heterodimerizing selectivity modules to accurately assemble orthogonal Fc-antigen binding domain constructs (constructs 45-48; FIG. 12-FIG. 15): (i) heterodimerizing selectivity modules having different reverse charge mutations (Table 4) and (ii) heterodimerizing selectivity modules having engineered cavities and protuberances (Table 3). Any heterodimerizing selectivity module can be incorporated into a pair of Fc monomers designed to assemble into a particular Fc domain of the construct by introducing specific amino acid substitutions into each Fc monomer polypeptide. The heterodimerizing selectivity modules are designed to encourage association between Fc monomers having the complementary amino acid substitutions of a particular heterodimerizing selectivity module, while disfavoring association with Fc monomers having the mutations of a different heterodimerizing selectivity module. These heterodimerizing mutations ensure the assembly of the different Fc monomer polypeptides into the desired tandem configuration of different Fc domains of a construct with minimal formation of smaller or larger complexes. The properties of these constructs allow for the efficient generation of substantially homogenous pharmaceutical compositions, which is desirable to ensure the safety, efficacy, uniformity, and reliability of the pharmaceutical compositions.

In some embodiments, assembly of an orthogonal Fc-antigen binding domain construct described herein can be accomplished using different electrostatic steering mutations between the two sets of heterodimerizing mutations as described herein. One example of orthogonal electrostatic steering mutations is E357K in a first knob of an Fc monomer and K370D in a first hole of an Fc monomer, wherein these Fc monomers associate to form a first Fc domain, and D399K in a second knob of an Fc monomer and K409D in a second hole of an Fc monomer, wherein these Fc monomers associate to form a second Fc domain.

In some embodiments, the Fc-antigen binding domain construct has at least two antigen-binding domains (e.g., two, three, four, five, or six antigen-binding domains) with different binding characteristics, such as different binding affinities (for the same or different targets) or specificities for different target molecules. Bispecific constructs may be generated from the above Fc scaffolds in which two or more of the polypeptides of the Fc-antigen binding domain construct include different antigen-binding domains, e.g., a long chain includes one antigen-binding domain of a first specificity and a short chain includes a different antigen-binding domain of a second specificity. The different antigen binding domains may use different light chains, or a common light chain, or may consist of scFv domains.

Bi-specific and tri-specific constructs may be generated by the use of two different sets of heterodimerizing mutations, i.e., orthogonal heterodimerizing mutations. Such heterodimerizing sequences need to be designed in such a way that they disfavor association with the other heterodimerizing sequences. Such designs can be accomplished using different electrostatic steering mutations between the two sets of heterodimerizing mutations, and/or different protuberance-into-cavity mutations between the two sets of heterodimerizing mutations, as described herein. One example of orthogonal electrostatic steering mutations is E357K in the first knob Fc, K370D in first hole Fc, D399K in the second knob Fc, and K409D in the second hole Fc.

### I. Fc Domain Monomers

An Fc domain monomer includes at least a portion of a hinge domain, a C_{H}2 antibody constant domain, and a C_{H}3 antibody constant domain (e.g., a human IgG1 hinge, a C_{H}2 antibody constant domain, and a C_{H}3 antibody constant domain with optional amino acid substitutions). The Fc domain monomer can be of immunoglobulin antibody isotype IgG, IgE, IgM, IgA, or IgD. The Fc domain monomer may also be of any immunoglobulin antibody isotype (e.g., IgG1, IgG2a, IgG2b, IgG3, or IgG4). The Fc domain monomers may also be hybrids, e.g., with the hinge and C_{H}2 from IgG1 and the C_{H}3 from IgA, or with the hinge and C_{H}2 from IgG1 but the C_{H}3 from IgG3. A dimer of Fc domain monomers is an Fc domain (further defined herein) that can bind to an Fc receptor, e.g., FcγRIIIa, which is a receptor located on the surface of leukocytes. In the present disclosure, the C_{H}3 antibody constant domain of an Fc domain monomer may contain amino acid substitutions at the interface of the C_{H}3-C_{H}3 antibody constant domains to promote their association with each other. In other embodiments, an Fc domain monomer includes an additional moiety, e.g., an albumin-binding peptide or a purification peptide, attached to the N- or C-terminus. In the present disclosure, an Fc domain monomer does not contain any type of antibody variable region, e.g., V_{H}, V_{L}, a complementarity determining region (CDR), or a hypervariable region (HVR).

In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of SEQ ID NO:42. In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of any one of SEQ ID NOs: 43, 44, 46, 47, 48, and 50-53. In certain embodiments, an Fc domain monomer in the Fc-antigen binding domain construct may have a sequence that is at least 95% identical (at least 97%, 99%, or 99.5% identical) to the sequence of any one of SEQ ID NOs: 48, 52, and 53.

SEQ ID NO: 42
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVYTLPPSRDELTKNQVSLTCLVKGFYPS
DIAVEWESNGQPENNYKTTPPVLDSDGSFFLYSKLTVDK
SRWQQGNVFSCSVMHEALHNHYTQKSLSLSPGK
SEQ ID NO: 44
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVCTLPPSRDELTKNQVSLSCAVKGFYPS
DIAVEWESNGQPENNYKTTPPVLDSDGSFFLVSKLTVDK
SRWQQGNVFSCSVMHEALHNHYTQKSLSLSPGK
SEQ ID NO: 46
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVCTLPPSRDELTKNQVSLSCAVKGFYPS
DIAVEWESNGQPENNYKTTPPVLDSDGSFFLVSKLTVDK
SRWQQGNVFSCSVMHEALHNHYTQKSLSLSPG
SEQ ID NO: 48
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVCTLPPSRDELTKNQVSLSCAVDGFYPS
DIAVEWESNGQPENNYKTTPPVLDSDGSFFLVSKLTVDK
SRWQQGNVFSCSVMHEALHNHYTQKSLSLSPG
SEQ ID NO: 50
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVYTLPPCRDELTKNQVSLWCLVKGFYPS
DIAVEVVESNGQPENNYKTTPPVLDSDGSFFLYSKLTVD
KSRWQQGNVFSCSVMHEALHNHYTQKSLSLSPGK
SEQ ID NO: 51
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVYTLPPSRDELTKNQVSLTCLVKGFYPS
DIAVEWESNGQPENNYKTTPPVLKSDGSFFLYSDLTVDK
SRWQQGNVFSCSVMHEALHNHYTQKSLSLSPGK
SEQ ID NO: 52
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVYTLPPSRDELTKNQVSLTCLVKGFYPS
DIAVEWESNGQPENNYKTTPPVLKSDGSFFLYSDLTVDK
SRWQQGNVFSCSVMHEALHNHYTQKSLSLSPG
SEQ ID NO: 53
DKTHTCPPCPAPELLGGPSVFLFPPKPKDTLMISRTPEV
TCVVVDVSHEDPEVKFNVVYVDGVEVHNAKTKPREEQYN
STYRVVSVLTVLHQDWLNGKEYKCKVSNKALPAPIEKTI
SKAKGQPREPQVYTLPPCRDKLTKNQVSLWCLVKGFYPS
DIAVEVVESNGQPENNYKTTPPVLDSDGSFFLYSKLTVD
KSRWQQGNVFSCSVMHEALHNHYTQKSLSLSPGK

### II. Fc Domains

As defined herein, an Fc domain includes two Fc domain monomers that are dimerized by the interaction between the C_{H}3 antibody constant domains. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

### III. Antigen Binding Domains

An antigen binding domain may be any protein or polypeptide that binds to a specific target molecule or set of target molecules. Antigen binding domains include one or more peptides or polypeptides that specifically bind a target molecule. Antigen binding domains may include the antigen binding domain of an antibody. In some embodiments, the antigen binding domain may be a fragment of an antibody or an antibody-construct, e.g., the minimal portion of the antibody that binds to the target antigen. An antigen binding domain may also be a synthetically engineered peptide that binds a target specifically such as a fibronectin-based binding protein (e.g., a FN3 monobody). In some embodiments, an antigen binding domain cab be a ligand or receptor. A fragment antigen-binding (Fab) fragment is a region on an antibody that binds to a target antigen. It is composed of one constant and one variable domain of each of the heavy and the light chain. A Fab fragment includes a V_{H}, V_{L}, C_{H}1 and C_{L} domains. The variable domains V_{H} and V_{L} each contain a set of 3 complementarity-determining regions (CDRs) at the amino terminal end of the monomer. The Fab fragment can be of immunoglobulin antibody isotype IgG, IgE, IgM, IgA, or IgD. The Fab fragment monomer may also be of any immunoglobulin antibody isotype (e.g., IgG1, IgG2a, IgG2b, IgG3, or IgG4). In some embodiments, a Fab fragment may be covalently attached to a second identical Fab fragment following protease treatment (e.g., pepsin) of an immunoglobulin, forming an F(ab′)₂ fragment. In some embodiments, the Fab may be expressed as a single polypeptide, which includes both the variable and constant domains fused, e.g. with a linker between the domains.

In some embodiments, only a portion of a Fab fragment may be used as an antigen binding domain. In some embodiments, only the light chain component (V_{L}+C_{L}) of a Fab may be used, or only the heavy chain component (V_{H}+C_{H}) of a Fab may be used. In some embodiments, a single-chain variable fragment (scFv), which is a fusion protein of the the V_{H} and V_{L} chains of the Fab variable region, may be used. In other embodiments, a linear antibody, which includes a pair of tandem Fd segments (V_{H}-C_{H}1-V_{H}-C_{H}1), which, together with complementary light chain polypeptides form a pair of antigen binding regions, may be used.

Antigen binding domains may be placed in various numbers and at various locations within the Fc-containing polypeptides described herein. In some embodiments, one or more antigen binding domains may be placed at the N-terminus, C-terminus, and/or in between the Fc domains of an Fc-containing polypeptide. In some embodiments, a polypeptide or peptide linker can be placed between an antigen binding domain, e.g., a Fab domain, and an Fc domain of an Fc-containing polypeptide. In some embodiments, multiple antigen binding domains (e.g., 2, 3, 4, or 5 or more antigen binding domains) joined in a series can be placed at any position along a polypeptide chain (Wu et al., Nat. Biotechnology, 25:1290-1297, 2007).

In some embodiments, two or more antigen binding domains can be placed at various distances relative to each other on an Fc-domain containing polypeptide or on a protein complex made of numerous Fc-domain containing polypeptides. In some embodiments, two or more antigen binding domains are placed near each other, e.g., on the same Fc domain, as in a monoclonal antibody). In some embodiments, two or more antigen binding domains are placed farther apart relative to each other, e.g., the antigen binding domains are separated from each other by 1, 2, 3, 4, or 5, or more Fc domains on the protein structure.

In some embodiments, an antigen binding domain of the present disclosure includes for a target or antigen listed in Table 1A and 1B, one, two, three, four, five, or all six of the CDR sequences listed in Table 1A and 1B for the listed target or antigen, as provided in further detail below Table 1A and 1B.

TABLE 1A
CDR Sequences

| | | CDR1- | CDR2- | CDR3- | CDR1- | CDR2- | CDR3- |
|---|---|---|---|---|---|---|---|
| | Antibody | IMGT | IMGT | IMGT | IMGT | IMGT | IMGT |
| Target | Name | (heavy) | (heavy) | (heavy) | (light) | (light) | (light) |
| B7-H3 | Enoblitzumab | GFTF | ISSD | GRGR | QNVDTN | SAS | QQYN |
| | | SSFG | SSAI | ENIY | (SEQ | | NYPF |
| | | (SEQ | (SEQ | YGSR | ID | | T |
| | | ID | ID | LDY | NO: | | (SEQ ID |
| | | NO: | NO: | (SEQ ID | 171) | | NO: |
| | | 76) | 106) | NO: | | | 201) |
| | | | | 137) | | | |
| beta- | Gantenerumab | GFTF | INAS | ARGK | QSVS | GAS | LQIYN |
| amyloid | | SSYA | GTRT | GNTH | SSY | | MPIT |
| | | (SEQ | (SEQ | KPYG | (SEQ | | (SEQ ID |
| | | ID | ID | YVRY | ID | | NO: |
| | | NO: | NO: | FDV | NO: | | 202) |
| | | 77) | 107) | (SEQ ID | 172) | | |
| | | | | NO: | | | |
| | | | | 138) | | | |
| CCR4 | Mogamulizumab | GFIFS | ISSA | GRHS | RNIVH | KVS | FQGSL |
| | | NYG | STYS | DGNF | INGDTY | | LPW |
| | | (SEQ | (SEQ | AFGY | (SEQ | | T |
| | | ID | ID | (SEQ ID | ID | | (SEQ ID |
| | | NO: | NO: | NO: | NO: | | NO: |
| | | 78) | 108) | 139) | 173) | | 203) |
| CD19 | Inebilizumab | GFTF | IYPG | ARSG | ESVDT | EAS | QQSK |
| | | SSSW | DGDT | FITTV | FGISF | | EVPF |
| | | (SEQ | (SEQ | RDFDY | (SEQ | | T |
| | | ID | ID | (SEQ ID | ID | | (SEQ ID |
| | | NO: | NO: | NO: | NO: | | NO: |
| | | 79) | 109) | 140) | 174) | | 204) |
| CD20 | Obinutuzumab | GYAF | IFPG | ARNV | KSLLH | QMS | AQNLE |
| | | SYSW | DGDT | FDGY | SNGITY | | LPYT |
| | | (SEQ | (SEQ | WLVY | (SEQ | | (SEQ ID |
| | | ID | ID | (SEQ ID | ID | | NO: |
| | | NO: | NO: | NO: | NO: | | 205) |
| | | 80) | 110) | 141) | 175) | | |
| CD20 | Ocaratuzumab | GRTF | AIYP | ARST | SSVPY | ATS | QQWL |
| | | TSYN | LTGD | YVGG | (SEQ | | SNPP |
| | | MH | T | DWQF | ID | | T |
| | | (SEQ | (SEQ | DV | NO: | | (SEQ ID |
| | | ID | ID | (SEQ | 176) | | NO: |
| | | NO: | NO: | ID | | | 206) |
| | | 81) | 111) | NO: | | | |
| | | | | 142) | | | |
| CD20 | Rituximab | GYTF | IYPG | CARST | SSVSY | ATS | QQWT |
| | | TSYN | NGDT | YYGGD | (SEQ | | SNPP |
| | | (SEQ | (SEQ | WYFNV | ID | | T |
| | | ID | ID | (SEQ | NO: | | (SEQ ID |
| | | NO: | NO: | ID | 177) | | NO: |
| | | 82) | 112) | NO: | | | 207) |
| | | | | 143) | | | |
| CD20 | Ublituximab | GYTF | IYPG | ARYDY | SSVSY | ATS | QQWT |
| | | TSYN | NGDT | NYAMDY | (SEQ | | FNPP |
| | | (SEQ | (SEQ | (SEQ | ID | | T |
| | | ID | ID | ID | NO: | | (SEQ ID |
| | | NO: | NO: | NO: | 177) | | NO: |
| | | 82) | 112) | 144) | | | 208) |
| CD20 | Veltuzumab | GYTF | IYPGN | ARSTY | SSVSY | ATS | QQWT |
| | | TSYN | GDT | YGGDW | (SEQ | | SNPP |
| | | (SEQ | (SEQ | YFDV | ID | | T |
| | | ID | ID | (SEQ | NO: | | (SEQ |
| | | NO: | NO: | ID | 177) | | ID |
| | | 82) | 112) | NO: | | | NO: |
| | | | | 145) | | | 207) |
| CD22 | Epratuzumab | GYTF | INPR | ARRDI | QSVLY | WAS | HQYLSS |
| | | TSYW | NDYT | TTFY | SANH | | (SEQ NO: |
| | | (SEQ | (SEQ | (SEQ | KNY | | 209) |
| | | ID | ID | ID | (SEQ | | |
| | | NO: | NO: | NO: | ID | | |
| | | 83) | 113) | 146) | NO: | | |
| | | | | | 178) | | |
| CD37 | Otlertuzumab | GYSF | IDPY | ARSV | ENVYSY | FAK | QHHS |
| | | TGYN | YGGT | GPFD | (SEQ | | DNPW |
| | | (SEQ | (SEQ | S | ID | | T |
| | | ID | ID | (SEQ | NO: | | (SEQ |
| | | NO: | NO: | ID | 179) | | ID |
| | | 84) | 114) | NO: | | | NO: |
| | | | | 147) | | | 210) |
| CD38 | Daratumumab | GFTF | ISGS | AKDK | QSVSSY | DAS | QQRS |
| | | NSFA | GGGT | ILWF | (SEQ | | NWPP |
| | | (SEQ | (SEQ | GEPV | ID | | T |
| | | ID | ID | FDY | NO: | | (SEQ |
| | | NO: | NO: | (SEQ | 180) | | ID |
| | | 85) | 115) | ID | | | NO: |
| | | | | NO: | | | 211) |
| | | | | 148) | | | |
| CD38 | Isatuximab | GYTF | IYPG | ARGD | QDVSTV | SAS | QQHY |
| | | TDYW | DGDT | YYGS | (SEQ | | SPPY |
| | | (SEQ | (SEQ | NSLD | ID | | T |
| | | ID | ID | Y | NO: | | (SEQ |
| | | NO: | NO: | (SEQ | 181) | | ID |
| | | 86) | 109) | ID | | | NO: |
| | | | | NO: | | | 212) |
| | | | | 149) | | | |
| CD3epsilon | Foralumab | GFKF | IWYD | ARQM | QSVSSY | DAS | QQRS |
| | | SGYG | GSKK | GYWH | (SEQ | | NWPP |
| | | (SEQ | (SEQ | FDLW | ID | | LT |
| | | ID | ID | (SEQ | NO: | | (SEQ |
| | | NO: | NO: | ID | 180) | | ID |
| | | 87) | 116) | NO: | | | NO: |
| | | | | 150) | | | 213) |
| CD52 | Alemtuzumab | GFTF | IRDK | AREG | QNIDKY | NTN | LQHI |
| | | TDFY | AKGY | HTAA | (SEQ | | SRPRT |
| | | (SEQ | TT | PFDY | ID | | (SEQ |
| | | ID | (SEQ | (SEQ | NO: | | ID |
| | | NO: | ID | ID | 182) | | NO: |
| | | 88) | NO: | NO: | | | 214) |
| | | | 117) | 151) | | | |
| CD105 | Carotuximab | GFTF | IRSK | TRWR | SSVSY | ATS | QQWS |
| | | SDAW | ASNH | RFFD | (SEQ | | SNPL |
| | | (SEQ | AT | S | ID | | T |
| | | ID | (SEQ | (SEQ | NO: | | (SEQ |
| | | NO: | ID | ID | 177) | | ID |
| | | 89) | NO: | NO: | | | NO: |
| | | | 118) | 152) | | | 215) |
| CD147 | cHAb18 | GFTF | IRSA | TRDS | QSVI | TAS | QQDT |
| | | SDAW | NNHA | TATH | ND | | SPP |
| | | (SEQ | PT | (SEQ | (SEQ | | (SEQ |
| | | ID | (SEQ | ID | ID | | ID |
| | | NO: | ID | NO: | NO: | | NO: |
| | | 89) | NO: | 153) | 183) | | 216) |
| | | | 119) | | | | |
| c-Met | ABT-700 | GYIF | IKPN | ARSE | ESVDS | RAS | QQSK |
| | | TAYT | NGLA | ITTE | YANSF | | EDPL |
| | | (SEQ | (SEQ | FDY | (SEQ | | T |
| | | ID | ID | (SEQ | ID | | (SEQ |
| | | NO: | NO: | ID | NO: | | ID |
| | | 90) | 120) | NO: | 184) | | NO: |
| | | | | 154) | | | 217) |
| CTLA-4 | Ipilimumab | GFTF | ISYD | ARTG | QSVG | GAF | QQYG |
| | | SSYT | GNNK | WLGP | SSY | | SSPW |
| | | (SEQ | (SEQ | FDY | (SEQ | | T |
| | | ID | ID | (SEQ | ID | | (SEQ |
| | | NO: | NO: | ID | NO: | | ID |
| | | 91) | 121) | NO: | 185) | | NO: |
| | | | | 155) | | | 218) |
| EGFR2 | Margetuximab | GFNI | IYPT | SRWG | QDVNTA | SAS | QQHY |
| | | KDTY | NGYT | GDGF | (SEQ | | TTPP |
| | | (SEQ | (SEQ | YAMD | ID | | T |
| | | ID | ID | Y | NO: | | (SEQ |
| | | NO: | NO: | (SEQ | 186) | | ID |
| | | 92) | 122) | ID | | | NO: |
| | | | | NO: | | | 219) |
| | | | | 156) | | | |
| EGFR3 | Lumretuzumab | GYTF | IYAG | ARHR | QSVL | WAS | QSDY |
| | | RSSY | TGSP | DYYS | NSGN | | SYPY |
| | | (SEQ | (SEQ | NSLT | QKNY | | T |
| | | ID | ID | Y | (SEQ | | (SEQ |
| | | NO: | NO: | (SEQ | ID | | ID |
| | | 93) | 123) | ID | NO: | | NO: |
| | | | | NO: | 187) | | 220) |
| | | | | 157) | | | |
| EphA3 | Ifabotuzumab | GYTF | IYPG | ARGG | QGIISY | AAS | GQYA |
| | | TGYW | SGNT | YYED | (SEQ | | NYPY |
| | | (SEQ | (SEQ | FDS | ID | | T |
| | | ID | ID | (SEQ | NO: | | (SEQ |
| | | NO: | NO: | ID | 188) | | ID |
| | | 94) | 124) | NO: | | | NO: |
| | | | | 158) | | | 221) |
| GD3 | Ecromeximab | GFAF | ISSG | TRVK | QDISNY | YSS | HQYS |
| | | SHYA | GSGT | LGTY | (SEQ | | KLP |
| | | (SEQ | (SEQ | YFDS | ID | | (SEQ |
| | | ID | ID | (SEQ | NO: | | ID |
| | | NO: | NO: | ID | 189) | | NO: |
| | | 95) | 125) | NO: | | | 222) |
| | | | | 159) | | | |
| GPC3 | Codrituzumab | GYTF | LDPK | TRFY | QSLV | KVS | SQNTH |
| | | TDYE | TGDT | SYTY | HSNR | | VPPT |
| | | (SEQ | (SEQ | (SEQ | NTY | | (SEQ |
| | | ID | ID | ID | (SEQ | | ID |
| | | NO: | NO: | NO: | ID | | NO: |
| | | 96) | 126) | 160) | NO: | | 223) |
| | | | | | 190) | | |
| KIR2DL1/2/3 | Lirilumab | GGTF | FIPI | ARIP | QSVSSY | DAS | QQRS |
| | | SFYA | FGAA | SGSY | (SEQ | | NWMY |
| | | (SEQ | (SEQ | YYDY | ID | | T |
| | | ID | ID | DMDV | NO: | | (SEQ |
| | | NO: | NO: | (SEQ | 180) | | ID |
| | | 97) | 127) | ID | | | NO: |
| | | | | NO: | | | 224) |
| | | | | 161) | | | |
| MUC5AC | Ensituximab | GFSL | IWGD | VKPG | SSISY | DTS | HQRD |
| | | SKFG | GST | GDY | (SEQ | | SYPW |
| | | (SEQ | (SEQ | (SEQ | ID | | T |
| | | ID | ID | ID | NO: | | (SEQ |
| | | NO: | NO: | NO: | 191) | | ID |
| | | 98) | 128) | 162) | | | NO: |
| | | | | | | | 225) |
| Phosphatidyl- | Bavituximab | GYSF | IDPY | VKGG | QDIGSS | ATS | LQYV |
| serine | | TGYN | YGDT | YYGH | (SEQ | | SSPP |
| | | (SEQ | (SEQ | WYFD | ID | | T |
| | | ID | ID | V | NO: | | (SEQ |
| | | NO: | NO: | (SEQ | 192) | | ID |
| | | 84) | 129) | ID | | | NO: |
| | | | | NO: | | | 226) |
| | | | | 163) | | | |
| RHD | Roledumab | GFTF | ISYD | ARPV | QDIRNY | AAS | QQYY |
| | | KNYA | GRNI | RSRW | (SEQ | | NSPP |
| | | (SEQ | (SEQ | LQLG | ID | | T |
| | | ID | ID | LEDA | NO: | | (SEQ |
| | | NO: | NO: | FHI | 193) | | ID |
| | | 99) | 130) | (SEQ | | | NO: |
| | | | | ID | | | 227) |
| | | | | NO: | | | |
| | | | | 164) | | | |
| SLAMF7 | Elotuzumab | GFDF | INPD | ARPD | QDVGIA | WAS | QQYS |
| | | SRYW | SSTI | GNYW | (SEQ | | SYPY |
| | | (SEQ | (SEQ | YFDV | ID | | T |
| | | ID | ID | (SEQ | NO: | | (SEQ |
| | | NO: | NO: | ID | 194) | | ID |
| | | 100) | 131) | NO: | | | NO: |
| | | | | 165) | | | 228) |
| HER2 | Trastuzumab | GFNI | IYPT | SRWG | QDVNTA | SAS | QQHY |
| | | KDTY | NGYT | GDGF | (SEQ | | TTPP |
| | | (SEQ | (SEQ | YAMD | ID | | T |
| | | ID | ID | Y | NO: | | (SEQ |
| | | NO: | NO: | (SEQ | 186) | | ID |
| | | 92) | 122) | ID | | | NO: |
| | | | | NO: | | | 219) |
| | | | | 156) | | | |
| OX40 | Oxelumab | GFTF | ISGS | AKDR | QGISSW | AAS | QQYN |
| | | NSYA | | LVAP | (SEQ | | SYPY |
| | | (SEQ | GGFT | GTFD | ID | | T |
| | | ID | (SEQ | Y | NO: | | (SEQ |
| | | NO: | ID | (SEQ | 195) | | ID |
| | | 101) | NO: | ID | | | NO: |
| | | | 132) | NO: | | | 229) |
| | | | | 166) | | | |
| PD-L1 | Avelumab | GFTF | IYPS | ARIK | SSDV | DVS | SSYT |
| | | SSYI | GGIT | LGTV | GGYN | | SSST |
| | | (SEQ | (SEQ | TTVD | Y | | RV |
| | | ID | ID | Y | (SEQ | | (SEQ |
| | | NO: | NO: | (SEQ | ID | | ID |
| | | 102) | 133) | ID | NO: | | NO: |
| | | | | NO: | 196) | | 230) |
| | | | | 167) | | | |
| CD135 | 4G8-SDIEM | SYWMH | EIDP | AITT | RASQS | YSQSIS | QQSN |
| | | (SEQ | SDSY | TPFD | ISNN | (SEQ | TWPY |
| | | ID | KDYN | F | LH | ID | T |
| | | NO: | QKFK | (SEQ | (SEQ | NO: | (SEQ |
| | | 103) | D | ID | ID | 200) | ID |
| | | | (SEQ | NO: | NO: | | NO: |
| | | | ID | 168) | 197) | | 231) |
| | | | NO: | | | | |
| | | | 134) | | | | |
| HIV1 | VRC01LS | GYTF | GWMK | ARYF | SQYG | GGS | QQYE |
| | | LNCPI | PRGG | FGSS | SLAW | | FFGQ |
| | | (SEQ | AVN | PNWY | (SEQ | | GT |
| | | ID | (SEQ | FD | ID | | (SEQ |
| | | NO: | ID | (SEQ | NO: | | ID |
| | | 104) | NO: | ID | 198) | | NO: |
| | | | 135) | NO: | | | 232) |
| | | | | 169) | | | |
| HER3 | KTN3379 | GFTF | IGSS | ARVG | SLSN | SRN | AAWD |
| | | SYYY | GGVT | LGDA | IGLN | | DSPP |
| | | MQ | N | FDIW | (SEQ | | G |
| | | (SEQ | (SEQ | QQ | ID | | (SEQ |
| | | ID | ID | (SEQ | NO: | | ID |
| | | NO: | NO: | ID | 199) | | NO: |
| | | 105) | 136) | NO: | | | 233) |
| | | | | 170) | | | |

TABLE 1B
Variable Domain Sequences

| Antibody | VH/CH1 | VL |
|---|---|---|
| Atezolizumab | EVQLVESGGGLVQPGGSL | DIQMTQSPSSLSASVGDR |
| PD-L1 | RLSCAASGFTFSDSWIHW | VTITCRASQDVSTAVAWY |
| | VRQAPGKGLEWVAWISPY | QQKPGKAPKLLIYSASFL |
| | GGSTYYADSVKGRFTISA | YSGVPSRFSGSGSGTDFT |
| | DTSKNTAYLQMNSLRAED | LTISSLQPEDFATYYCQQ |
| | TAVYCARRHWPGGFDYWG | YLYHPATFGQGTKVEIKR |
| | QGTLVTVSSASTKGPSVF | TVAAPSVFIFPPSDEQLK |
| | PLAPSSKSTSGGTAALGC | SGTASWCLLNNFYPREAK |
| | LVKDYFPEPVTVSWNSGA | VQWKVDNALQSGNSQESV |
| | LTSGVHTFPAVLQSSGLY | TEQDSKDSTYSLSSTLTL |
| | SLSSVVTVPSSSLGTQTYI | SKADYEKHKVYACEVTHQ |
| | CNVNHKPSNTKVDKKVEP | GLSSPVTKSFNRGEC |
| | KSCDKTHTCPPCPAPELL | (SEQ ID NO: 266) |
| | GGPSVFLFPPKPKDTLMI | |
| | SRTPEVTCVVVDVSHEDP | |
| | EVKFNWYVDGVEVHNAKT | |
| | KPREEQYASTYRVVSVLT | |
| | VLHQDWLNGKEYKCKVSN | |
| | KALPAPIEKTISKAKGQP | |
| | REPQVYTLPPSREEMTKN | |
| | QVSLTCLVKGFYPSDIAV | |
| | EWESNGQPENNYKTTPPV | |
| | LDSDGSFFLYSKLTVDKS | |
| | RWQQGNVFSCSVMHEALH | |
| | NHYTQKSLSLSPGK | |
| | (SEQ ID NO: 261) | |
| Durvalumab | EVQLVESGGGLVQPGGSL | EIVLTQSPGTLSLSPGER |
| PD-L1 | RLSCAASGFTFSRYWMSW | ATLSCRASQRVSSSYLAW |
| | VRQAPGKGLEWVANIKQD | YQQKPGQAPRLLIYDASS |
| | GSEKYYVDSVKGRFTISR | RATGIPDRFSGSGSGTDF |
| | DNAKNSLYLQMNSLRAED | TLTISRLEPEDFAVYYCQ |
| | TAVYYCAREGGWFGELAF | QYGSLPWTFGQGTKVEIK |
| | DYWGQGTLVTVSSASTKG | RTVAAPSVFIFPPSDEQL |
| | PSVFPLAPSSKSTSGGTA | KSGTASVVCLLNNFYPRE |
| | ALGCLVKDYFPEPVTVSW | AKVQWKVDNALQSGNSQE |
| | NSGALTSGVHTFPAVLQS | SVTEQDSKDSTYSLSSTL |
| | SGLYSLSSVVTVPSSSLGT | TLSKADYEKHKVYACEVT |
| | QTYICNVNHKPSNTKVDK | HQGLSSPVTKSFNRGEC |
| | RVEPKSCDKTHTCPPCPA | (SEQ ID NO: 267) |
| | PEFEGGPSVFLFPPKPKD | |
| | TLMISRTPEVTCWVDVSH | |
| | EDPEVKFNWYVDGVEVHN | |
| | AKTKPREEQYNSTYRVVS | |
| | VLTVLHQDWLNGKEYKCK | |
| | VSNKALPASIEKTISKAK | |
| | GQPREPQVYTLPPSREEM | |
| | TKNQVSLTCLVKGFYPSD | |
| | IAVEWESNGQPENNYKTT | |
| | PPVLDSDGSFFLYSKLTV | |
| | DKSRWQQGNVFSCSVMHE | |
| | ALHNHYTQKSLSLSPGK | |
| | (SEQ ID NO: 262) | |
| Tremelimumab | QVQLVESGGG WQPGRSLRL | DIQMTQSPSSLSASV |
| CTLA-4 | SCAASGFTFS SYGMHWVRQA | GDRVTITCRASQSIN |
| | PGKGLEWVAV IWYDGSNKYY | SYLDWYQQKPGKAPKLL |
| | ADSVKGRFTI SRDNSKNTLY | IYAASSLQSGVPSRFSG |
| | LQMNSLRAED TAVYYCARDP | SGSGTDFTLTISSLQPE |
| | RGATLYYYYY GMDVWGQGTT | DFATYYCQQYYSTPFTF |
| | VTVSSASTKG PSVFPLAPCS | GPGTKVEIKRTVAAPSV |
| | RSTSESTAAL GCLVKDYFPE | FIFPPSDEQLKSGTASW |
| | PVTVSWNSGA LTSGVHTFPA | CLLNNFYPREAKVQWKV |
| | VLQSSGLYSL SSVVTVPSSN | DNALQSGNSQESVTEQD |
| | FGTQTYTCNV DHKPSNTKVD | SKDSTYSLSSTLTLSKA |
| | KTVERKCCVE CPPCPAPPVA | DYEKHKVYACEVTHQGL |
| | GPSVFLFPPK PKDTLMISRT | SSPVTKSFNRGEC |
| | PEVTCVVVDV SHEDPEVQFN | (SEQ ID NO: 268) |
| | WYVDGVEVHN AKTKPREEQF | |
| | NSTFRVVSVL TVVHQDWLNG | |
| | KEYKCKVSNK GLPAPIEKTI | |
| | SKTKGQPREP QVYTLPPSRE | |
| | EMTKNQVSLT CLVKGFYPSD | |
| | IAVEWESNGQ PENNYKTTPP | |
| | MLDSDGSFFL YSKLTVDKSR | |
| | WQQGNVFSCS VMHEALHNHY | |
| | TQKSLSLSPG K | |
| | (SEQ ID NO: 263) | |
| Isatuximab CD38 | QVQLVQSGAEVAKPGTSVKL | DIVMTQSHLSMSTSLGDP |
| | SCKASGYTFTDYWMQWVKQR | VSITCKASQDVSTVVAWY |
| | PGQGLEWIGTIYPGDGDTGY | QQKPGQSPRRLIYSASYR |
| | AQKFQGKATLTADKSSKTVY | YIGVPDRFTGSGAGTDFT |
| | MHLSSLASEDSAVYYCARGD | FTISSVQAEDLAVYYCQQ |
| | YYGSNSLDYWGQGTSVTVSS | HYSPPYTFGGGTKLEIKR |
| | ASTKGPSVFPLAPSSKSTSG | TVAAPSVFIFPPSDEQLK |
| | GTAALGCLVKDYFPEPVTVS | SGTASVVCLLNNFYPREA |
| | WNSGALTSGVHTFPAVLQSS | KVQWKVDNALQSGNSQES |
| | GLYSLSSVVTVPSSSLGTQT | VTEQDSKDSTYSLSSTLT |
| | YICNVNHKPSNTKVDKKVEP | LSKADYEKHKVYACEVTH |
| | KSCDKTHTCPPCPAPELLGG | QGLSSPVTKSFNRGEC |
| | PSVFLFPPKPKDTLMISRTP | (SEQ ID NO: 269) |
| | EVTCVWDVSHEDPEVKFNWY | |
| | VDGVEVHNAKTKPREEQYNS | |
| | TYRVVSVLTVLHQDWLNGKE | |
| | YKCKVSNKALPAPIEKTISK | |
| | AKGQPREPQVYTLPPSRDEL | |
| | TKNQVSLTCLVKGFYPSDIA | |
| | VEWESNGQPENNYKTTPPVL | |
| | DSDGSFFLYSKLTVDKSRWQ | |
| | QGNVFSCSVMHEALHNHYTQ | |
| | KSLSLSPGK | |
| | (SEQ ID NO: 264) | |
| MOR 202 CD38 | QVQLVESGGGLVQPGGSLRLS | DIELTQPPSVSVAPGQTA |
| | CAASGFTFSSYYMNVWRQAPG | RISCSGDNLRHYYVYWYQ |
| | KGLEVWSGISGDPSNTYYADS | QKPGQAPVLVIYGDSKRP |
| | VKGRFTISRDNSKNTLYLQMN | SGIPERFSGSNSGNTATL |
| | SLRAEDTAVYYCARDLPLVYT | TISGTQAEDEADYYCQTY |
| | GFAYWGQGTLVTV | TGGASLVFGGGTKLTVLGQ |
| | (SEQ ID NO: 265) | (SEQ ID NO: 270) |
| | (VH Only) | |

The antigen binding domains of Fc-antigen binding domain construct 45 (4504/4512 and 4518/4520 in FIG. 12) each can include the three heavy chain and the three light chain CDR sequences of any one of the antibodies listed in Table 1A and 1B.

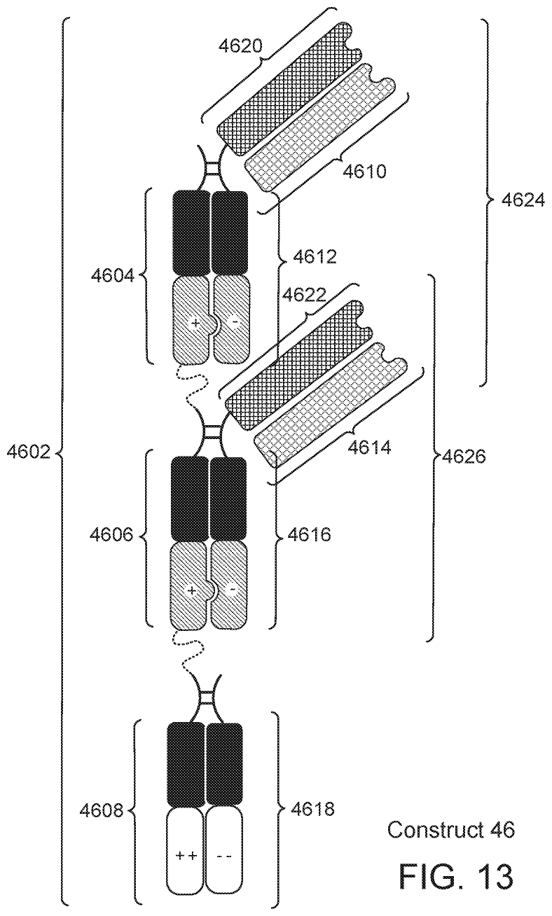

The antigen binding domains of Fc-antigen binding domain construct 46 (4610/4620 and 4614/4622 in FIG. 13) each can include the three heavy chain and the three light chain CDR sequences of any one of the antibodies listed in Table 1A and 1B.

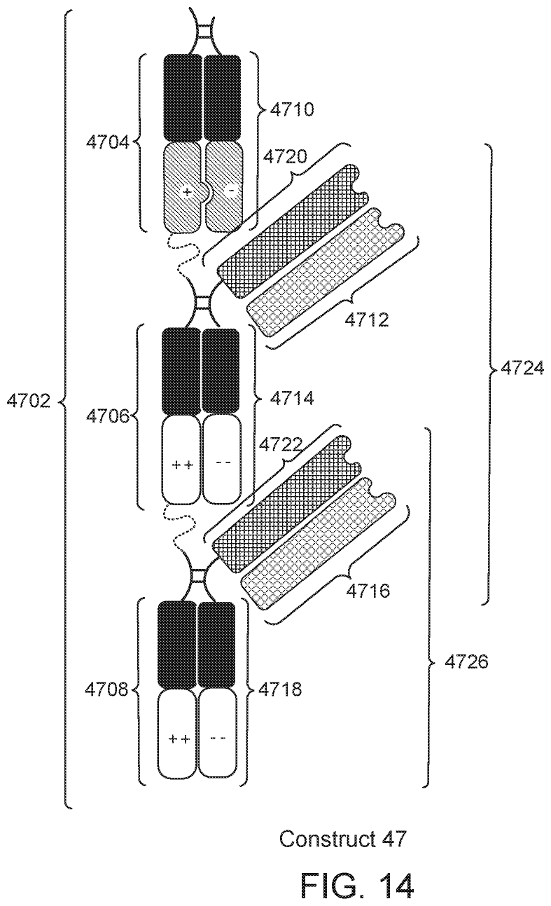

The antigen binding domains of Fc-antigen binding domain construct 47 (4712/4720 and 4716/4722 in FIG. 14) each can include the three heavy chain and the three light chain CDR sequences of any one of the antibodies listed in Table 1A and 1B.

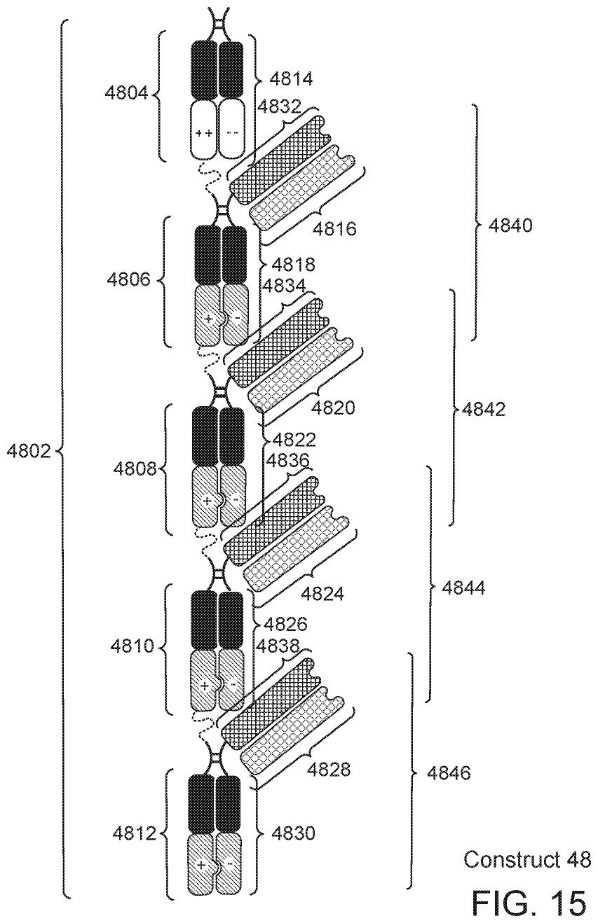

The antigen binding domains of Fc-antigen binding domain construct 48 (4816/4832, 4820/4834, 4824/4836, and 4828/4838 in FIG. 15) each can include the three heavy chain and the three light chain CDR sequences of any one of the antibodies listed in Table 1A and 1B.

In some embodiments, the antigen binding domain (e.g., a Fab or a scFv) includes the V_{H} and V_{L} chains of an antibody listed in Table 2 or Table 1B. In some embodiments, the Fab includes the CDRs contained in the V_{H} and V_{L} chains of an antibody listed in Table 2 or Table 1B. In some embodiments, the Fab includes the CDRs contained in the V_{H} and V_{L} chains of an antibody listed in Table 2 and the remainder of the V_{H} and V_{L} sequences are at least 95% identical, at least 97% identical, at least 99% identical, or at least 99.5% identical to the V_{H} and V_{L} sequences of an antibody in Table 2. In some embodiments, the Fab includes the CDRs contained in the V_{H} and V_{L} chains of an antibody listed in Table 1B and the remainder of the V_{H} and V_{L} sequences are at least 95% identical, at least 97% identical, at least 99% identical, or at least 99.5% identical to the V_{H} and V_{L} sequences of an antibody in Table 1B.

TABLE 2

| Target | Antibody Name |
|---|---|
| AbGn-7 antigen | AbGn-7 |
| AMHR2 | GM-102 |
| B7-H3 | DS-5573a |
| CA19-9 | MVT-5873 |
| CAIX | Anti-CAIX |
| CD19 | XmAb5871 |
| CD33 | BI-836858 |
| CD37 | BI-836826 |
| CD38 | MOR-202 |
| CD47 | Anti-CD47 |
| CD70 | ARGX-110 |
| CD70 | ARGX-110 |
| CD98 | IGN-523 |
| CD147 | Metuzumab |
| CD157 | MEN-1112 |
| c-Met | ARGX-111 |
| EGFR2 | GT-Mab 7.3-GEX |
| EphA2 | DS-8895a |
| FGFR2 | FPA-144 |
| GM2 | BIW-8962 |
| HPA-1a | NAITgam |
| ICAM-1 | BI-505 |
| IL-3Ralpha | Talacotuzumab |
| JL-1 | Leukotuximab |
| kappa myeloma | MDX-1097 |
| antigen | |
| KIR32DL2 | IPH-4102 |
| LAG-3 | GSK-2381781 |
| P. aeruginosa | AR-104 |
| serotype O1 | |
| pGlu-abeta | PBD-C06 |
| TA-MUC1 | GT-MAB 2.5-GEX |

The antigen binding domains of Fc-antigen binding domain construct 45 (4504/4512 and 4518/4520 in FIG. 12) each can include the V_{H} and V_{L} sequences of any one of the antibodies listed in Table 2 or Table 1B.

The antigen binding domains of Fc-antigen binding domain construct 46 (4610/4620 and 4614/4622 in FIG. 13) each can include the V_{H} and V_{L} sequences of any one of the antibodies listed in Table 2 or Table 1B.

The antigen binding domains of Fc-antigen binding domain construct 47 (4712/4720 and 4716/4722 in FIG. 14) each can include the V_{H} and V_{L} sequences of any one of the antibodies listed in Table 2 or Table 1B.

The antigen binding domains of Fc-antigen binding domain construct 48 (4816/4832, 4820/4834, 4824/4836, and 4828/4838 in FIG. 15) each can include the V_{H} and V_{L} sequences of any one of the antibodies listed in Table 2 or Table 1B.

The antigen binding domains of Fc-antigen binding domain construct 45 (4504/4512 and 4518/4520 in FIG. 12) each can include the CDR sequences contained in the V_{H} and V_{L} sequences of any one of the antibodies listed in Table 2 or Table 1B.

The antigen binding domains of Fc-antigen binding domain construct 46 (4610/4620 and 4614/4622 in FIG. 13) each can include the CDR sequences contained in the V_{H} and V_{L} sequences of any one of the antibodies listed in Table 2 or Table 1B.

The antigen binding domains of Fc-antigen binding domain construct 47 (4712/4720 and 4716/4722 in FIG. 14) each can include the CDR sequences contained in the V_{H} and V_{L} sequences of any one of the antibodies listed in Table 2 or Table 1B.

The antigen binding domains of Fc-antigen binding domain construct 48 (4816/4832, 4820/4834, 4824/4836, and 4828/4838 in FIG. 15) each can include the CDR sequences contained in the V_{H} and V_{L} sequences of any one of the antibodies listed in Table 2 or Table 1B.

The antigen binding domains of Fc-antigen binding domain construct 45 (4504/4512 and 4518/4520 in FIG. 12) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2 or Table 1B.

The antigen binding domains of Fc-antigen binding domain construct 46 (4610/4620 and 4614/4622 in FIG. 13) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2 or Table 1B.

The antigen binding domains of Fc-antigen binding domain construct 47 (4712/4720 and 4716/4722 in FIG. 14) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2 or Table 1B.

The antigen binding domains of Fc-antigen binding domain construct 48 (4816/4832, 4820/4834, 4824/4836, and 4828/4838 in FIG. 15) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2 or Table 1B.

### IV. Dimerization Selectivity Modules

In the present disclosure, a dimerization selectivity module includes components or select amino acids within the Fc domain monomer that facilitate the preferred pairing of two Fc domain monomers to form an Fc domain. Specifically, a dimerization selectivity module is that part of the C_{H}3 antibody constant domain of an Fc domain monomer which includes amino acid substitutions positioned at the interface between interacting C_{H}3 antibody constant domains of two Fc domain monomers. In a dimerization selectivity module, the amino acid substitutions make favorable the dimerization of the two C_{H}3 antibody constant domains as a result of the compatibility of amino acids chosen for those substitutions. The ultimate formation of the favored Fc domain is selective over other Fc domains which form from Fc domain monomers lacking dimerization selectivity modules or with incompatible amino acid substitutions in the dimerization selectivity modules. This type of amino acid substitution can be made using conventional molecular cloning techniques well-known in the art, such as QuikChange® mutagenesis.

In some embodiments, a dimerization selectivity module includes an engineered cavity (described further herein) in the C_{H}3 antibody constant domain. In other embodiments, a dimerization selectivity module includes an engineered protuberance (described further herein) in the C_{H}3 antibody constant domain. To selectively form an Fc domain, two Fc domain monomers with compatible dimerization selectivity modules, e.g., one C_{H}3 antibody constant domain containing an engineered cavity and the other C_{H}3 antibody constant domain containing an engineered protuberance, combine to form a protuberance-into-cavity pair of Fc domain monomers. Engineered protuberances and engineered cavities are examples of heterodimerizing selectivity modules, which can be made in the C_{H}3 antibody constant domains of Fc domain monomers in order to promote favorable heterodimerization of two Fc domain monomers that have compatible heterodimerizing selectivity modules.

In other embodiments, an Fc domain monomer with a dimerization selectivity module containing positively-charged amino acid substitutions and an Fc domain monomer with a dimerization selectivity module containing negatively-charged amino acid substitutions may selectively combine to form an Fc domain through the favorable electrostatic steering (described further herein) of the charged amino acids. In some embodiments, an Fc domain monomer may include one or more of the following positively-charged and negatively-charged amino acid substitutions: K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids. In another example, an Fc domain monomer containing E356K and D399K and an Fc domain monomer containing K392D and K409D may selectively combine to form an Fc domain through favorable electrostatic steering of the charged amino acids. In some embodiments, reverse charge amino acid substitutions may be used as heterodimerizing selectivity modules, wherein two Fc domain monomers containing different, but compatible, reverse charge amino acid substitutions combine to form a heterodimeric Fc domain. Specific dimerization selectivity modules are further listed, without limitation, in Tables 3 and 4 described further below.

In other embodiments, two Fc domain monomers include homodimerizing selectivity modules containing identical reverse charge mutations in at least two positions within the ring of charged residues at the interface between C_{H}3 domains. Homodimerizing selectivity modules are reverse charge amino acid substitutions that promote the homodimerization of Fc domain monomers to form a homodimeric Fc domain. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. In one embodiment, an Fc domain includes Fc domain monomers including the double mutants K409D/D399K, K392D/D399K, E357K/K370E, D356K/K439D, K409E/D399K, K392E/D399K, E357K/K370D, or D356K/K439E. In another embodiment, an Fc domain includes Fc domain monomers including quadruple mutants combining any pair of the double mutants, e.g., K409D/D399K/E357K/K370E. Examples of homodimerizing selectivity modules are further shown in Tables 5 and 6. Homodimerizing Fc domains can be used to create symmetrical branch points on an Fc-antigen binding domain construct. In one embodiment, an Fc-antigen binding domain construct described herein has one homodimerizing Fc domain. In one embodiment, an Fc-antigen binding domain construct has two or more homodimerizing Fc domains, e.g., two, three, four, or five or more homodimerizing domains. In one embodiment, an Fc-antigen binding domain construct has three homodimerizing Fc domains. In some embodiments, an Fc-antigen binding domain construct has one homodimerizing selectivity module. In some embodiments, an Fc-antigen binding domain construct has two or more homodimerizing selectivity modules, e.g., two, three, four, or five or more homodimerizing selectivity modules.

In further embodiments, an Fc domain monomer containing (i) at least one reverse charge mutation and (ii) at least one engineered cavity or at least one engineered protuberance may selectively combine with another Fc domain monomer containing (i) at least one reverse charge mutation and (ii) at least one engineered protuberance or at least one engineered cavity to form an Fc domain. For example, an Fc domain monomer containing reversed charge mutation K370D and engineered cavities Y349C, T366S, L368A, and Y407V and another Fc domain monomer containing reversed charge mutation E357K and engineered protuberances S354C and T366W may selectively combine to form an Fc domain.

The formation of such Fc domains is promoted by the compatible amino acid substitutions in the C_{H}3 antibody constant domains. Two dimerization selectivity modules containing incompatible amino acid substitutions, e.g., both containing engineered cavities, both containing engineered protuberances, or both containing the same charged amino acids at the C_{H}3-C_{H}3 interface, will not promote the formation of a heterodimeric Fc domain.

Multiple pairs of heterodimerizing Fc domains can be used to create Fc-antigen binding domain constructs with multiple asymmetrical branch points, multiple non-branching points, or both asymmetrical branch points and non-branching points. Multiple, distinct heterodimerization technologies (see, e.g., Tables 3 and 4) are incorporated into different Fc domains to assemble these Fc domain-containing constructs. The heterodimerization technologies have minimal association (orthogonality) for undesired pairing of Fc monomers. Two different Fc heterodimerization methods, such as knobs-into-holes (Table 3) and electrostatic steering (Table 4), can be used in different Fc domains to control the assembly of the polypeptide chains into the desired construct. Alternatively, two different variants of knobs-into-holes (e.g., two distinct sets of mutations selected from Table 3), or two different variants of electrostatic steering (e.g., two distinct sets of mutations selected from Table 4), can be used in different Fc domains to control the assembly of the polypeptide chains into the desired construct. Asymmetrical branches can be created by placing the Fc domain monomers of a heterodimerizing Fc domain on different polypeptide chains, polypeptide chain having multiple Fc domains. Non-branching points can be created by placing one Fc domain monomer of the heterodimerizing Fc domain on a polypeptide chain with multiple Fc domains and the other Fc domain monomer of the heterodimerizing Fc domain on a polypeptide chain with a single Fc domain.

In some embodiments, the Fc-antigen binding domain constructs described herein are linear. In some embodiments, the Fc-antigen binding domain constructs described herein do not have branch points. For example, an Fc-antigen binding domain construct can be assembled from one large peptide with two or more Fc domain monomers, wherein at least two Fc domain monomers are different (i.e., have different heterodimerizing mutations), and two or more smaller peptides, each having a different single Fc domain monomer (i.e., two or more small peptides with Fc domain monomers having different heterodimerizing mutations). The Fc-antigen binding domain constructs described herein can have two or more dimerization selectivity modules that are incompatible with each other, e.g., at least two incompatible dimerization selectivity modules selected from Tables 3 and/or 4, that promote or facilitate the proper formation of the Fc-antigen binding domain constructs, so that the Fc domain monomer of each smaller peptide associates with its compatible Fc domain monomer(s) on the large peptide. In some embodiments, a first Fc domain monomer or first subset of Fc domain monomers on a long peptide contains amino acids substitutions forming part of a first dimerization selectivity module that is compatible to a part of the first dimerization selectivity module formed by amino acid substitutions in the Fc domain monomer of a first short peptide. A second Fc domain monomer or second subset of Fc domain monomers on the long peptide contains amino acids substitutions forming part of a second dimerization selectivity module that is compatible to part of the second dimerization selectivity module formed by amino acid substitutions in the Fc domain monomer of a second short peptide. The first dimerization selectivity module favors binding of a first Fc domain monomer (or first subset of Fc domain monomers) on the long peptide to the Fc domain monomer of a first short peptide, while disfavoring binding between a first Fc domain monomer and the Fc domain monomer of the second short peptide. Similarly, the second dimerization selectivity module favors binding of a second Fc domain monomer (or second subset of Fc domain monomers) on the long peptide to the Fc domain monomer of the second short peptide, while disfavoring binding between a second Fc domain monomer and the Fc domain monomer of the first short peptide.

In certain embodiments, an Fc-antigen binding domain construct can have a first Fc domain with a first dimerization selectivity module, and a second Fc domain with a second dimerization selectivity module. In some embodiments, the first Fc domain is assembled from one Fc monomer with at least one protuberance-forming mutations selected from Table 3 and/or at least one reverse charge mutation selected from Table 4 (e.g., the Fc monomer can have S354C and T366W protuberance-forming mutations and an E357K reverse charge mutation), and one Fc monomer with at least one cavity-forming mutation from selected from Table 3 and/or at least one reverse charge mutation selected from Table 4 (e.g., the Fc monomer can have Y349C, T366S, L368A, and Y407V cavity-forming mutations and a K370D reverse charge mutation. In some embodiments, the second Fc domain is assembled from one Fc monomer with at least one protuberance-forming mutations selected from Table 3 and/or at least one reverse charge mutation selected from Table 4 (e.g., the Fc monomer can have D356K and D399K reverse charge mutations), and one Fc monomer with at least one cavity-forming mutation from selected from Table 3 and/or at least one reverse charge mutation selected from Table 4 (e.g., the Fc monomer can have K392D and K409D reverse charge mutations).

Furthermore, other methods used to promote the formation of Fc domains with defined Fc domain monomers include, without limitation, the LUZ-Y approach (U.S. Patent Application Publication No. WO2011034605) which includes C-terminal fusion of a monomer α-helices of a leucine zipper to each of the Fc domain monomers to allow heterodimer formation, as well as strand-exchange engineered domain (SEED) body approach (Davis et al., Protein Eng Des Sel. 23:195-202, 2010) that generates Fc domain with heterodimeric Fc domain monomers each including alternating segments of IgA and IgG C_{H}3 sequences.

### V. Engineered Cavities and Engineered Protuberances

The use of engineered cavities and engineered protuberances (or the “knob-into-hole” strategy) is described by Carter and co-workers (Ridgway et al., Protein Eng. 9:617-612, 1996; Atwell et al., J Mol Biol. 270:26-35, 1997; Merchant et al., Nat Biotechnol. 16:677-681, 1998). The knob and hole interaction favors heterodimer formation, whereas the knob-knob and the hole-hole interaction hinder homodimer formation due to steric clash and deletion of favorable interactions. The “knob-into-hole” technique is also disclosed in U.S. Pat. No. 5,731,168.

In the present disclosure, engineered cavities and engineered protuberances are used in the preparation of the Fc-antigen binding domain constructs described herein. An engineered cavity is a void that is created when an original amino acid in a protein is replaced with a different amino acid having a smaller side-chain volume. An engineered protuberance is a bump that is created when an original amino acid in a protein is replaced with a different amino acid having a larger side-chain volume. Specifically, the amino acid being replaced is in the C_{H}3 antibody constant domain of an Fc domain monomer and is involved in the dimerization of two Fc domain monomers. In some embodiments, an engineered cavity in one C_{H}3 antibody constant domain is created to accommodate an engineered protuberance in another C_{H}3 antibody constant domain, such that both C_{H}3 antibody constant domains act as dimerization selectivity modules (e.g., heterodimerizing selectivity modules) (described above) that promote or favor the dimerization of the two Fc domain monomers. In other embodiments, an engineered cavity in one C_{H}3 antibody constant domain is created to better accommodate an original amino acid in another C_{H}3 antibody constant domain. In yet other embodiments, an engineered protuberance in one C_{H}3 antibody constant domain is created to form additional interactions with original amino acids in another C_{H}3 antibody constant domain.

An engineered cavity can be constructed by replacing amino acids containing larger side chains such as tyrosine or tryptophan with amino acids containing smaller side chains such as alanine, valine, or threonine. Specifically, some dimerization selectivity modules (e.g., heterodimerizing selectivity modules) (described further above) contain engineered cavities such as Y407V mutation in the C_{H}3 antibody constant domain. Similarly, an engineered protuberance can be constructed by replacing amino acids containing smaller side chains with amino acids containing larger side chains. Specifically, some dimerization selectivity modules (e.g., heterodimerizing selectivity modules) (described further above) contain engineered protuberances such as T366W mutation in the C_{H}3 antibody constant domain. In the present disclosure, engineered cavities and engineered protuberances are also combined with inter-C_{H}3 domain disulfide bond engineering to enhance heterodimer formation. In one example, an Fc domain monomer containing engineered cavities Y349C, T366S, L368A, and Y407V may selectively combine with another Fc domain monomer containing engineered protuberances S354C and T366W to form an Fc domain. In another example, an Fc domain monomer containing an engineered cavity with the addition of Y349C and an Fc domain monomer containing an engineered protuberance with the addition of S354C may selectively combine to form an Fc domain. Other engineered cavities and engineered protuberances, in combination with either disulfide bond engineering or structural calculations (mixed HA-TF) are included, without limitation, in Table 3.

TABLE 3
Fc heterodimerization methods (Knobs-into-holes)

| | Mutations | Mutations | |
|---|---|---|---|
| | (Chain A) | (Chain B) | |
| | (CH3 domain | (CH₃ domain | |
| | of Fc domain | of Fc domain | |
| Method | monomer 1 | monomer 2 | Reference |
| Knobs-into- | Y407T | T336Y | US Pat. # |
| Holes (Y-T) | | | 8,216,805 |
| Knobs-into- | Y407A | T336W | US Pat. # |
| Holes | | | 8,216,805 |
| Knobs-into- | F405A | T394W | US Pat. # |
| Holes | | | 8,216,805 |
| Knobs-into- | Y407T | T366Y | US Pat. # |
| Holes | | | 8,216,805 |
| Knobs-into- | T394S | F405W | US Pat. # |
| Holes | | | 8,216,805 |
| Knobs-into- | T394W, Y407T | T366Y, F406A | US Pat. # |
| Holes | | | 8,216,805 |
| Knobs-into- | T394S, Y407A | T366W, F405W | US Pat. # |
| Holes | | | 8,216,805 |
| Knobs-into- | T366W, T394S | F405W, T407A | US Pat. # |
| Holes | | | 8,216,805 |
| Knobs-into- | F405T | T394Y | |
| Holes | | | |
| Knobs-into- | S354C, T366W | Y349C, T366S, | |
| Holes | | L368A, Y407V | |
| Knobs-into- | Y349C, T366S, | S354C, T366W | Merchant et al., |
| Holes (CW- | L368A, Y407A | | Nat. Biotechnol. |
| CSAV) | | | 16(7): 677-81, |
| | | | 1998 |
| HA-TF | S364H, F405A | Y349T, T394F | WO2011028952 |

Note:
All residues numbered per the EU numbering scheme (Edelman et al, Proc Natl Acad Sci USA, 63: 78-85, 1969)

Replacing an original amino acid residue in the C_{H}3 antibody constant domain with a different amino acid residue can be achieved by altering the nucleic acid encoding the original amino acid residue. The upper limit for the number of original amino acid residues that can be replaced is the total number of residues in the interface of the C_{H}3 antibody constant domains, given that sufficient interaction at the interface is still maintained.

Combining Engineered Cavities and Engineered Protuberances with Electrostatic Steering

Electrostatic steering can be combined with knob-in-hole technology to favor heterominerization, for example, between Fc domain monomers in two different polypeptides. Electrostatic steering, described in greater detail below, is the utilization of favorable electrostatic interactions between oppositely charged amino acids in peptides, protein domains, and proteins to control the formation of higher ordered protein molecules. Electrostatic steering can be used to promote either homodimerization or heterodimerization, the latter of which can be usefully combined with knob-in-hole technology. In the case of heterodimerization, different, but compatible, mutations are introduced in each of the Fc domain monomers which are to heterodimerize. Thus, an Fc domain monomer can be modified to include one of the following positively-charged and negatively-charged amino acid substitutions: D356K, D356R, E357K, E357R, K370D, K370E, K392D, K392E, D399K, K409D, K409E, K439D, and K439E. For example, one Fc domain monomer, for example, an Fc domain monomer having a cavity (Y349C, T366S, L368A and Y407V), can also include K370D mutation and the other Fc domain monomer, for example, an Fc domain monomer having a protuberance (S354C and T366W) can include E357K.

More generally, any of the cavity mutations (or mutation combinations): Y407T, Y407A, F405A, Y407T, T394S, T394W:Y407A, T366W:T394S, T366S:L368A:Y407V:Y349C, and S3364H:F405 can be combined with a mutation in Table 4 and any of the protuberance mutations (or mutation combinations): T366Y, T366W, T394W, F405W, T366Y:F405A, T366W:Y407A, T366W:S354C, and Y349T:T394F can be combined with a mutation in Table 4 that is paired with the Table 4 mutation used in combination with the cavity mutation (or mutation combination).

More generally, any of the cavity mutations (or mutation combinations): Y407T, Y407A, F405A, Y407T, T394S, T394W:Y407A, T366W:T394S, T366S:L368A:Y407V:Y349C, and S3364H:F405 can be combined with an electrostatic steering mutation in Table 3 and any of the protuberance mutations (or mutation combinations): T366Y, T366W, T394W, F405W, T366Y:F405A, T366W:Y407A, T366W:S354C, and Y349T:T394F can be combined with an electrostatic steering mutation in Table 3.

### VI. Electrostatic Steering

Electrostatic steering is the utilization of favorable electrostatic interactions between oppositely charged amino acids in peptides, protein domains, and proteins to control the formation of higher ordered protein molecules. A method of using electrostatic steering effects to alter the interaction of antibody domains to reduce for formation of homodimer in favor of heterodimer formation in the generation of bi-specific antibodies is disclosed in U.S. Patent Application Publication No. 2014-0024111.

In the present disclosure, electrostatic steering is used to control the dimerization of Fc domain monomers and the formation of Fc-antigen binding domain constructs. In particular, to control the dimerization of Fc domain monomers using electrostatic steering, one or more amino acid residues that make up the C_{H}3-C_{H}3 interface are replaced with positively- or negatively-charged amino acid residues such that the interaction becomes electrostatically favorable or unfavorable depending on the specific charged amino acids introduced. In some embodiments, a positively-charged amino acid in the interface, such as lysine, arginine, or histidine, is replaced with a negatively-charged amino acid such as aspartic acid or glutamic acid. In other embodiments, a negatively-charged amino acid in the interface is replaced with a positively-charged amino acid. The charged amino acids may be introduced to one of the interacting C_{H}3 antibody constant domains, or both. By introducing charged amino acids to the interacting C_{H}3 antibody constant domains, dimerization selectivity modules (described further above) are created that can selectively form dimers of Fc domain monomers as controlled by the electrostatic steering effects resulting from the interaction between charged amino acids.

In some embodiments, to create a dimerization selectivity module including reversed charges that can selectively form dimers of Fc domain monomers as controlled by the electrostatic steering effects, the two Fc domain monomers may be selectively formed through heterodimerization or homodimerization.

Heterodimerization of Fc Domain Monomers

Heterodimerization of Fc domain monomers can be promoted by introducing different, but compatible, mutations in the two Fc domain monomers, such as the charge residue pairs included, without limitation, in Table 4. In some embodiments, an Fc domain monomer may include one or more of the following positively-charged and negatively-charged amino acid substitutions: D356K, D356R, E357K, E357R, K370D, K370E, K392D, K392E, D399K, K409D, K409E, K439D, and K439E, e.g., 1, 2, 3, 4, or 5 or more of D356K, D356R, E357K, E357R, K370D, K370E, K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids. In another example, an Fc domain monomer containing E356K and D399K and an Fc domain monomer containing K392D and K409D may selectively combine to form an Fc domain through favorable electrostatic steering of the charged amino acids.

A “heterodimeric Fc domain” refers to an Fc domain that is formed by the heterodimerization of two Fc domain monomers, wherein the two Fc domain monomers contain different reverse charge mutations (heterodimerizing selectivity modules) (see, e.g., mutations in Table 4) that promote the favorable formation of these two Fc domain monomers. In one example, in an Fc-antigen binding domain construct having three Fc domains, two of the three Fc domains may be formed by the heterodimerization of two Fc domain monomers, as promoted by the electrostatic steering effects.

TABLE 4
Fc heterodimerization methods (electrostatic steering)

| | Mutations | Mutations | |
|---|---|---|---|
| | (Chain A) | (Chain B) | |
| | (CH3 domain | (CH₃ domain | |
| | of Fc domain | of Fc domain | |
| Method | monomer 1 | monomer 2 | Reference |
| Electrostatic | K409D | D399K | US 2014/0024111 |
| Steering | | | |
| Electrostatic | K409D | D399R | US 2014/0024111 |
| Steering | | | |
| Electrostatic | K409E | D399K | US 2014/0024111 |
| Steering | | | |
| Electrostatic | K409E | D399R | US 2014/0024111 |
| Steering | | | |
| Electrostatic | K392D | D399K | US 2014/0024111 |
| Steering | | | |
| Electrostatic | K392D | D399R | US 2014/0024111 |
| Steering | | | |
| Electrostatic | K392E | D399K | US 2014/0024111 |
| Steering | | | |
| Electrostatic | K392E | D399R | US 2014/0024111 |
| Steering | | | |
| Electrostatic | K392D, | E356K, | Gunasekaran et |
| Steering (DD- | K409D | D399K | al., J Biol Chem. |
| KK) | | | 285: 19637-46, |
| | | | 2010 |
| Electrostatic | K370E, | E356K, | WO 2006/106905 |
| Steering | K409D, | E357K, | |
| | K439E | D399K | |
| Knobs-into- | S354C, | Y349C, | WO 2015/168643 |
| Holes plus | E357K, | T366S, | |
| Electrostatic | T366W | L368A, | |
| Steering | | K370D, | |
| | | Y407V | |
| Electrostatic | K370D | E357K | US 2014/0024111 |
| Steering | | | |
| Electrostatic | K370D | E357R | US 2014/0024111 |
| Steering | | | |
| Electrostatic | K370E | E357K | US 2014/0024111 |
| Steering | | | |
| Electrostatic | K370E | E357R | US 2014/0024111 |
| Steering | | | |
| Electrostatic | K370D | D356K | US 2014/0024111 |
| Steering | | | |
| Electrostatic | K370D | D356R | US 2014/0024111 |
| Steering | | | |
| Electrostatic | K370E | D356K | US 2014/0024111 |
| Steering | | | |
| Electrostatic | K370E | D356R | US 2014/0024111 |
| Steering | | | |
| Electrostatic | K370E, | E356K, | US 2014/0024111 |
| Steering | K409D, | E357K, | |
| | K439E | D399K | |

Note:
All residues numbered per the EU numbering scheme (Edelman et al, Proc Natl Acad Sci USA, 63: 78-85, 1969)

Homodimerization of Fc Domain Monomers

Homodimerization of Fc domain monomers can be promoted by introducing the same electrostatic steering mutations (homodimerizing selectivity modules) in both Fc domain monomers in a symmetric fashion. In some embodiments, two Fc domain monomers include homodimerizing selectivity modules containing identical reverse charge mutations in at least two positions within the ring of charged residues at the interface between C_{H}3 domains. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. Electrostatic steering mutations that may be introduced into an Fc domain monomer to promote its homodimerization are shown, without limitation, in Tables 5 and 6. In one embodiment, an Fc domain includes two Fc domain monomers each including the double reverse charge mutants (Table 5), e.g., K409D/D399K. In another embodiment, an Fc domain includes two Fc domain monomers each including quadruple reverse mutants (Table 6), e.g., K409D/D399K/K370D/E357K.

For example, in an Fc-antigen binding domain construct having three Fc domains, one of the three Fc domains may be formed by the homodimerization of two Fc domain monomers, as promoted by the electrostatic steering effects. A “homodimeric Fc domain” refers to an Fc domain that is formed by the homodimerization of two Fc domain monomers, wherein the two Fc domain monomers contain the same reverse charge mutations (see, e.g., mutations in Tables 5 and 6). In an Fc-antigen binding domain construct having three Fc domains—one carboxyl terminal “stem” Fc domain and two amino terminal “branch” Fc domains—the carboxy terminal “stem” Fc domain may be a homodimeric Fc domain (also called a “stem homodimeric Fc domain”). A stem homodimeric Fc domain may be formed by two Fc domain monomers each containing the double mutants K409D/D399K.

TABLE 5
Fc homodimerization methods

| | Mutations | |
|---|---|---|
| | (Chains A and B) | |
| | (CH3 domain of | |
| | Fc domain | |
| Method | monomers 1 and 2) | Reference |
| Wild Type | None | |
| Electrostatic | D399K, K409D | Gunasekaran et al., J Biol |
| Steering (KD) | | Chem. 258: 19637-46, 2010, |
| | | WO 2015/168643 |
| Electrostatic | D399K, K409E | Gunasekaran et al., J Biol |
| Steering | | Chem. 285: 19637-46, 2010, |
| | | WO 2015/168643 |
| Electrostatic | E357K, K370D | Gunasekaran et al., J Biol |
| Steering | | Chem. 285: 19637-46, 2010, |
| | | WO 2015/168643 |
| Electrostatic | E357K, K370E | Gunasekaran et al., J Biol |
| Steering | | Chem. 285: 19637-46, 2010, |
| | | WO 2015/168643 |
| Electrostatic | D356K, K439D | Gunasekaran et al., J Biol |
| Steering | | Chem. 285: 19637-46, 2010, |
| | | WO 2015/168643 |
| Electrostatic | D356K, K439E | Gunasekaran et al., J Biol |
| Steering | | Chem. 285: 19637-46, 2010, |
| | | WO 2015/168643 |
| Electrostatic | K392D, D399K | Gunasekaran et al., J Biol |
| Steering | | Chem. 285: 19637-46, 2010, |
| | | WO 2015/168643 |
| Electrostatic | K392E, D399K | Gunasekaran et al., J Biol |
| Steering | | Chem. 285: 19637-46, 2010, |
| | | WO 2015/168643 |
| Electrostatic | D399R, K409D | |
| Steering | | |
| Electrostatic | D399R, K409E | |
| Steering | | |
| Electrostatic | D399R, K392D | |
| Steering | | |
| Electrostatic | D399R, K392E | |
| Steering | | |
| Electrostatic | E357K, K370D | |
| Steering | | |
| Electrostatic | E357R, K370D | |
| Steering | | |
| Electrostatic | E357K, K370E | |
| Steering | | |
| Electrostatic | E357R, K370E | |
| Steering | | |
| Electrostatic | D356K, K370D | |
| Steering | | |
| Electrostatic | D356R, K370D | |
| Steering | | |
| Electrostatic | D356K, K370E | |
| Steering | | |
| Electrostatic | D356R, K370E | |
| Steering | | |

Note:
All residues numbered per the EU numbering scheme (Edelman et al, Proc Natl Acad Sci USA, 63: 78-85, 1969)

TABLE 6
Fc homodimerization mutation-Four reverse charge

| Reverse charge mutations in C_{H}3 | Reverse charge mutations in C_{H}3 |
|---|---|
| constant domain of each of the | domain of each of the two Fc |
| two Fc domain monomers in a | domain monomers in a |
| homodimeric Fc domain | homodimeric Fc domain |
| K409D/D399K/K370D/E357K | K392D/D399K/K370D/E357K |
| K409D/D399K/K370D/E357R | K392D/D399K/K370D/E357R |
| K409D/D399K/K370E/E357K | K392D/D399K/K370E/E357K |
| K409D/D399K/K370E/E357R | K392D/D399K/K370E/E357R |
| K409D/D399K/K370D/D356K | K392D/D399K/K370D/D356K |
| K409D/D399K/K370D/D356R | K392D/D399K/K370D/D356R |
| K409D/D399K/K370E/D356K | K392D/D399K/K370E/D356K |
| K409D/D399K/K370E/D356R | K392D/D399K/K370E/D356R |
| K409D/D399R/K370D/E357K | K392D/D399R/K370D/E357K |
| K409D/D399R/K370D/E357R | K392D/D399R/K370D/E357R |
| K409D/D399R/K370E/E357K | K392D/D399R/K370E/E357K |
| K409D/D399R/K370E/E357R | K392D/D399R/K370E/E357R |
| K409D/D399R/K370D/D356K | K392D/D399R/K370D/D356K |
| K409D/D399R/K370D/D356R | K392D/D399R/K370D/D356R |
| K409D/D399R/K370E/D356K | K392D/D399R/K370E/D356K |
| K409D/D399R/K370E/D356R | K392D/D399R/K370E/D356R |
| K409E/D399K/K370D/E357K | K392E/D399K/K370D/E357K |
| K409E/D399K/K370D/E357R | K392E/D399K/K370D/E357R |
| K409E/D399K/K370E/E357K | K392E/D399K/K370E/E357K |
| K409E/D399K/K370E/E357R | K392E/D399K/K370E/E357R |
| K409E/D399K/K370D/D356K | K392E/D399K/K370D/D356K |
| K409E/D399K/K370D/D356R | K392E/D399K/K370D/D356R |
| K409E/D399K/K370E/D356K | K392E/D399K/K370E/D356K |
| K409E/D399K/K370E/D356R | K392E/D399K/K370E/D356R |
| K409E/D399R/K370D/E357K | K392E/D399R/K370D/E357K |
| K409E/D399R/K370D/E357R | K392E/D399R/K370D/E357R |
| K409E/D399R/K370E/E357K | K392E/D399R/K370E/E357K |
| K409E/D399R/K370E/E357R | K392E/D399R/K370E/E357R |
| K409E/D399R/K370D/D356K | K392E/D399R/K370D/D356K |
| K409E/D399R/K370D/D356R | K392E/D399R/K370D/D356R |
| K409E/D399R/K370E/D356K | K392E/D399R/K370E/D356K |
| K409E/D399R/K370E/D356R | K392E/D399R/K370E/D356R |

Note:
All residues numbered per the EU numbering scheme (Edelman et al, Proc Natl Acad Sci USA, 63: 78-85, 1969)

Other Heterodimerization Methods

Numerous other heterodimerization technologies have been described. Any one or more of these technologies (Table 7) can be combined with any knobs-into-holes and/or electrostatic steering heterodimerization and/or homodimerization technology described herein to make an Fc-antigen binding domain construct.

TABLE 7
Other Fc heterodimerization methods

| | Mutations | Mutations | |
|---|---|---|---|
| Method | (Chain A) | (Chain B) | Reference |
| ZW1 | T350V, L351Y, | T350V, T366L, | Von Kreudenstein |
| (VYAV- | F405A, Y407V | K392L, T394W | et al, MAbs, 5: |
| VLLW) | | | 646-54, 2013 |
| IgG1 | D221E, P228E, | D221R, P228R, | Strop et al, J Mol |
| hinge/CH3 | L368E | K409R | Biol, 420: 204-19, |
| charge pairs | | | 2012 |
| (EEE-RRR) | | | |
| EW-RVT | K360E, K409W | Q347R, D399V, | Choi et al, Mol |
| | | F405T | Cancer Ther, |
| | | | 12: 2748-59, |
| | | | 2013 |
| EW-RVT_{S-S} | K360E, K409W, | Q347R, D399V, | Choi et al, Mol |
| | Y349C | F405T, S354C | Immunol, 65: |
| | | | 377-83, 2015 |
| Charge | L351D | T366K | De Nardis, J Biol |
| Introduction | | | Chem, 292: |
| (DK | | | 14706-17, 2017 |
| Biclonic) | | | |
| Charge | L361D, L368E | L351K, T366K | De Nardis, J Biol |
| Introduction | | | Chem, 292: |
| (DEKK | | | 14706-17, 2017 |
| Biclonic) | | | |
| DuoBody | F405L | K409R | Labrijn et al, |
| (L-R) | | | Proc Natl Acad |
| | | | Sci USA, 110: |
| | | | 5145-50, 2013 |
| SEEDbody | IgG/A chimera | IgG/A chimera | Davis et al, |
| | | | Protein Eng Des |
| | | | Sel, 23: 195-202, |
| | | | 2010 |
| BEAT | S364K, T366V, | Q347E, Y349A, | Skegro et al, J |
| (A/B) | K370T, K392Y, | L351F, S364T, | Biol Chem, 292: |
| | F405S, Y407V, | T366V, K370T, | 9745-59, 2017 |
| | K409W, T411N | T394D, V397L, | |
| | | D399E, F405A, | |
| | | Y407S, K409R, | |
| | | T411R | |
| BEAT | S364K, T366V, | F405A, Y407S | Skegro et al, J |
| (A/B min) | K370T, K392Y, | | Biol Chem, 292: |
| | K409W, T411N | | 9745-59, 2017 |
| BEAT | Q347A, S364K, | Q347E, Y349A, | Skegro et al, J |
| (A/B + Q) | T366V, K370T, | L351F, S364T, | Biol Chem, 292: |
| | K392Y, F405S, | T366V, K370T, | 9745-59, 2017 |
| | Y407V, K409W, | T394D, V397L, | |
| | T411N | D399E, F405A, | |
| | | Y407S, K409R, | |
| | | T411R | |
| BEAT | S364T, T366V, | Q347E, Y349A, | Skegro et al, J |
| (A/B − T) | K370T, K392Y, | L351F, S364T, | Biol Chem, 292: |
| | F405S, Y407V, | T366V, K370T, | 9745-59, 2017 |
| | K409W, T411N | T394D, V397L, | |
| | | D399E, F405A, | |
| | | Y407S, K409R | |
| 7.8.60 | K360D, D399M, | E345R, Q347R, | Leaver-Fay et al, |
| (DMA- | Y407A | T366V, K409V | Structure, 24: |
| RRVV) | | | 641-51, 2016 |
| 20.8.34 | Y349S, K370Y, | E356G, E357D, | Leaver-Fay et al, |
| (SYMV- | T366M, K409V | S364Q, Y407A | Structure, 24: |
| GDQA) | | | 641-51, 2016 |

Note:
All residues numbered per the EU numbering scheme (Edelman et al, Proc Natl Acad Sci USA, 63: 78-85, 1969)

### VII. Linkers

In the present disclosure, a linker is used to describe a linkage or connection between polypeptides or protein domains and/or associated non-protein moieties. In some embodiments, a linker is a linkage or connection between at least two Fc domain monomers, for which the linker connects the C-terminus of the C_{H}3 antibody constant domain of a first Fc domain monomer to the N-terminus of the hinge domain of a second Fc domain monomer, such that the two Fc domain monomers are joined to each other in tandem series. In other embodiments, a linker is a linkage between an Fc domain monomer and any other protein domains that are attached to it. For example, a linker can attach the C-terminus of the C_{H}3 antibody constant domain of an Fc domain monomer to the N-terminus of an albumin-binding peptide.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two Fc domain monomer, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

Spacer

In the present disclosure, a linker between two Fc domain monomers can be an amino acid spacer including 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids). In some embodiments, a linker between two Fc domain monomers is an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between two Fc domain monomers is an amino acid spacer containing 12-30 amino acids (e.g., 12, 13, 14, 15, 16, 17, 18, 19, 20, 21, 22, 23, 24, 25, 26, 27, 28, 29, or 30 amino acids). Suitable peptide spacers are known in the art, and include, for example, peptide linkers containing flexible amino acid residues such as glycine and serine. In certain embodiments, a spacer can contain motifs, e.g., multiple or repeating motifs, of GS, GGS, GGGGS (SEQ ID NO: 1), GGSG (SEQ ID NO: 2), or SGGG (SEQ ID NO: 3). In certain embodiments, a spacer can contain 2 to 12 amino acids including motifs of GS, e.g., GS, GSGS (SEQ ID NO: 4), GSGSGS (SEQ ID NO: 5), GSGSGSGS (SEQ ID NO: 6), GSGSGSGSGS (SEQ ID NO: 7), or GSGSGSGSGSGS (SEQ ID NO: 8). In certain other embodiments, a spacer can contain 3 to 12 amino acids including motifs of GGS, e.g., GGS, GGSGGS (SEQ ID NO: 9), GGSGGSGGS (SEQ ID NO: 10), and GGSGGSGGSGGS (SEQ ID NO: 11). In yet other embodiments, a spacer can contain 4 to 20 amino acids including motifs of GGSG (SEQ ID NO: 2), e.g., GGSGGGSG (SEQ ID NO: 12), GGSGGGSGGGSG (SEQ ID NO: 13), GGSGGGSGGGSGGGSG (SEQ ID NO: 14), or GGSGGGSGGGSGGGSGGGSG (SEQ ID NO: 15). In other embodiments, a spacer can contain motifs of GGGGS (SEQ ID NO: 1), e.g., GGGGSGGGGS (SEQ ID NO: 16) or GGGGSGGGGSGGGGS (SEQ ID NO: 17). In certain embodiments, a spacer is SGGGSGGGSGGGSGGGSGGG (SEQ ID NO: 18).

In some embodiments, a spacer between two Fc domain monomers contains only glycine residues, e.g., at least 4 glycine residues (e.g., 4-200 (SEQ ID NO: 271), 4-180 (SEQ ID NO: 272), 4-160 (SEQ ID NO: 273), 4-140 (SEQ ID NO: 274), 4-40 (SEQ ID NO: 275), 4-100 (SEQ ID NO: 276), 4-90 (SEQ ID NO: 277), 4-80 (SEQ ID NO: 278), 4-70 (SEQ ID NO: 279), 4-60 (SEQ ID NO: 280), 4-50 (SEQ ID NO: 281), 4-40 (SEQ ID NO: 275), 4-30 (SEQ ID NO: 250), 4-20 (SEQ ID NO: 251), 4-19 (SEQ ID NO: 282), 4-18 (SEQ ID NO: 283), 4-17 (SEQ ID NO: 284), 4-16 (SEQ ID NO: 285), 4-15 (SEQ ID NO: 286), 4-14 (SEQ ID NO: 287), 4-13 (SEQ ID NO: 288), 4-12 (SEQ ID NO: 289), 4-11 (SEQ ID NO: 290), 4-10 (SEQ ID NO: 291), 4-9 (SEQ ID NO: 292), 4-8 (SEQ ID NO: 293), 4-7 (SEQ ID NO: 294), 4-6 (SEQ ID NO: 295) or 4-5 (SEQ ID NO: 296) glycine residues) (e.g., 4-200 (SEQ ID NO: 271), 6-200 (SEQ ID NO: 297), 8-200 (SEQ ID NO: 298), 10-200 (SEQ ID NO: 299), 12-200 (SEQ ID NO: 300), 14-200 (SEQ ID NO: 301), 16-200 (SEQ ID NO: 302), 18-200 (SEQ ID NO: 303), 20-200 (SEQ ID NO: 304), 30-200 (SEQ ID NO: 305), 40-200 (SEQ ID NO: 306), 50-200 (SEQ ID NO: 307), 60-200 (SEQ ID NO: 308), 70-200 (SEQ ID NO: 309), 80-200 (SEQ ID NO: 310), 90-200 (SEQ ID NO: 311), 100-200 (SEQ ID NO: 312), 120-200 (SEQ ID NO: 313), 140-200 (SEQ ID NO: 314), 160-200 (SEQ ID NO: 315), 180-200 (SEQ ID NO: 316), or 190-200 (SEQ ID NO: 317) glycine residues). In certain embodiments, a spacer has 4-30 (SEQ ID NO: 250) glycine residues (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues (SEQ ID NO: 250)). In some embodiments, a spacer containing only glycine residues may not be glycosylated (e.g., O-linked glycosylation, also referred to as O-glycosylation) or may have a decreased level of glycosylation (e.g., a decreased level of 0-glycosylation) (e.g., a decreased level of O-glycosylation with glycans such as xylose, mannose, sialic acids, fucose (Fuc), and/or galactose (Gal) (e.g., xylose)) as compared to, e.g., a spacer containing one or more serine residues (e.g., SGGGSGGGSGGGSGGGSGGG (SEQ ID NO: 18)).

In some embodiments, a spacer containing only glycine residues may not be O-glycosylated (e.g., O-xylosylation) or may have a decreased level of O-glycosylation (e.g., a decreased level of O-xylosylation) as compared to, e.g., a spacer containing one or more serine residues (e.g., SGGGSGGGSGGGSGGGSGGG (SEQ ID NO: 18)).

In some embodiments, a spacer containing only glycine residues may not undergo proteolysis or may have a decreased rate of proteolysis as compared to, e.g., a spacer containing one or more serine residues (e.g., SGGGSGGGSGGGSGGGSGGG (SEQ ID NO: 18)).

In certain embodiments, a spacer can contain motifs of GGGG (SEQ ID NO: 19), e.g., GGGGGGGG (SEQ ID NO: 20), GGGGGGGGGGGG (SEQ ID NO: 21), GGGGGGGGGGGGGGGG (SEQ ID NO: 22), or GGGGGGGGGGGGGGGGGGGG (SEQ ID NO: 23). In certain embodiments, a spacer can contain motifs of GGGGG (SEQ ID NO: 24), e.g., GGGGGGGGGG (SEQ ID NO: 25), or GGGGGGGGGGGGGGG (SEQ ID NO: 26). In certain embodiments, a spacer is GGGGGGGGGGGGGGGGGGGG (SEQ ID NO: 27).

In other embodiments, a spacer can also contain amino acids other than glycine and serine, e.g., GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), or GGSGGGSEGGGSEGGGSEGGGSEGGGSEGGGSGGGS (SEQ ID NO: 34).

In certain embodiments in the present disclosure, a 12- or 20-amino acid peptide spacer is used to connect two Fc domain monomers in tandem series, the 12- and 20-amino acid peptide spacers consisting of sequences GGGSGGGSGGGS (SEQ ID NO: 35) and SGGGSGGGSGGGSGGGSGGG (SEQ ID NO: 18), respectively. In other embodiments, an 18-amino acid peptide spacer consisting of sequence GGSGGGSGGGSGGGSGGS (SEQ ID NO: 36) may be used.

In some embodiments, a spacer between two Fc domain monomers may have a sequence that is at least 75% identical (e.g., at least 77%, 79%, 81%, 83%, 85%, 87%, 89%, 91%, 93%, 95%, 97%, 99%, or 99.5% identical) to the sequence of any one of SEQ ID NOs: 1-36 described above. In certain embodiments, a spacer between two Fc domain monomers may have a sequence that is at least 80% identical (e.g., at least 82%, 85%, 87%, 90%, 92%, 95%, 97%, 99%, or 99.5% identical) to the sequence of any one of SEQ ID NOs: 17, 18, 26, and 27. In certain embodiments, a spacer between two Fc domain monomers may have a sequence that is at least 80% identical (e.g., at least 82%, 85%, 87%, 90%, 92%, 95%, 97%, 99%, or 99.5%) to the sequence of SEQ ID NO: 18 or 27.

In certain embodiments, the linker between the amino terminus of the hinge of an Fc domain monomer and the carboxy terminus of a Fc monomer that is in the same polypeptide (i.e., the linker connects the C-terminus of the C_{H}3 antibody constant domain of a first Fc domain monomer to the N-terminus of the hinge domain of a second Fc domain monomer, such that the two Fc domain monomers are joined to each other in tandem series) is a spacer having 3 or more amino acids rather than a covalent bond (e.g., 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids) or an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids)). A spacer can also be present between the N-terminus of the hinge domain of a Fc domain monomer and the carboxy terminus of a CD38 binding domain (e.g., a CH1 domain of a CD38 heavy chain binding domain or the CL domain of a CD38 light chain binding domain) such that the domains are joined by a spacer of 3 or more amino acids (e.g., 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids) or an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids)).

### VII. Serum Protein-Binding Peptides

Binding to serum protein peptides can improve the pharmacokinetics of protein pharmaceuticals, and in particular the Fc-antigen binding domain constructs described here may be fused with serum protein-binding peptides

As one example, albumin-binding peptides that can be used in the methods and compositions described here are generally known in the art. In one embodiment, the albumin binding peptide includes the sequence DICLPRWGCLW (SEQ ID NO: 37). In some embodiments, the albumin binding peptide has a sequence that is at least 80% identical (e.g., 80%, 90%, or 100% identical) to the sequence of SEQ ID NO: 37.

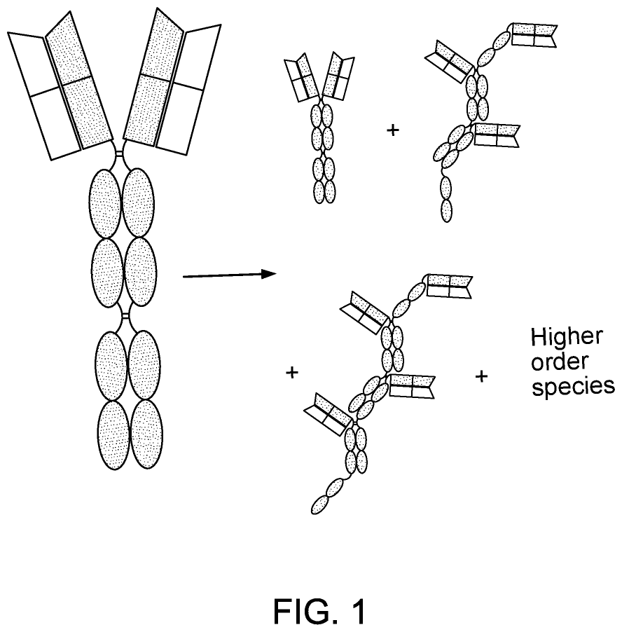

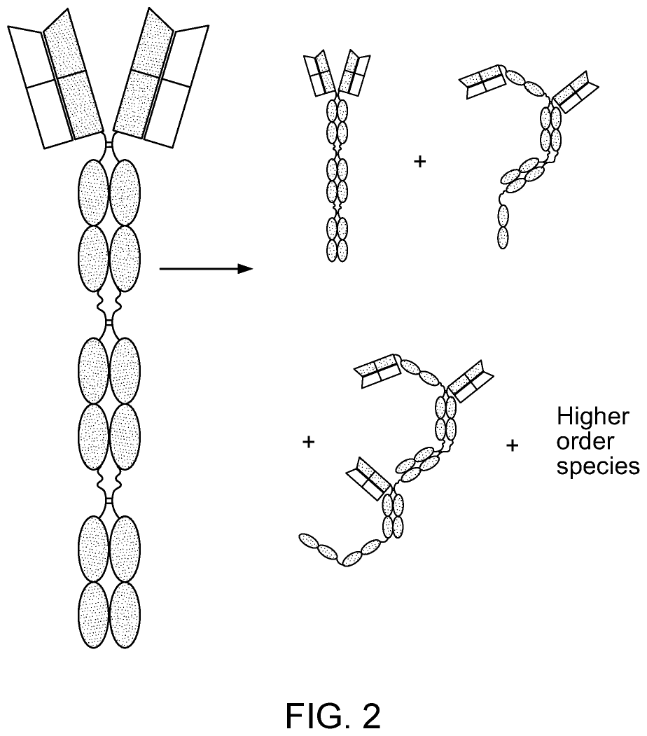

In the present disclosure, albumin-binding peptides may be attached to the N- or C-terminus of certain polypeptides in the Fc-antigen binding domain construct. In one embodiment, an albumin-binding peptide may be attached to the C-terminus of one or more polypeptides in Fc constructs containing an antigen binding domain. In another embodiment, an albumin-binding peptide can be fused to the C-terminus of the polypeptide encoding two Fc domain monomers linked in tandem series in Fc constructs containing an antigen binding domain. In yet another embodiment, an albumin-binding peptide can be attached to the C-terminus of Fc domain monomer (e.g., Fc domain monomers 114 and 116 in FIG. 1; Fc domain monomers 214 and 216 in FIG. 2) which is joined to the second Fc domain monomer in the polypeptide encoding the two Fc domain monomers linked in tandem series. Albumin-binding peptides can be fused genetically to Fc-antigen binding domain constructs or attached to Fc-antigen binding domain constructs through chemical means, e.g., chemical conjugation. If desired, a spacer can be inserted between the Fc-antigen binding domain construct and the albumin-binding peptide. Without being bound to a theory, it is expected that inclusion of an albumin-binding peptide in an Fc-antigen binding domain construct of the disclosure may lead to prolonged retention of the therapeutic protein through its binding to serum albumin.

### VIII. Fc-Antigen Binding Domain Constructs

In general, the disclosure features Fc-antigen binding domain constructs having 2-10 Fc domains and one or more antigen binding domains attached. These may have greater binding affinity and/or avidity than a single wild-type Fc domain for an Fc receptor, e.g., FcγRIIIa. The disclosure discloses methods of engineering amino acids at the interface of two interacting C_{H}3 antibody constant domains such that the two Fc domain monomers of an Fc domain selectively form a dimer with each other, thus preventing the formation of unwanted multimers or aggregates. An Fc-antigen binding domain construct includes an even number of Fc domain monomers, with each pair of Fc domain monomers forming an Fc domain. An Fc-antigen binding domain construct includes, at a minimum, two functional Fc domains formed from dimer of four Fc domain monomers and one antigen binding domain. The antigen binding domain may be joined to an Fc domain e.g., with a linker, a spacer, a peptide bond, a chemical bond or chemical moiety. In some embodiments, the disclosure relates to methods of engineering one set of amino acid substitutions selected from Tables 3 and 4 at the interface of a first pair of two interacting CH3 antibody constant domains, and engineering a second set of amino acid substitutions selected from Tables 3 and 4, different from the first set of amino acid substitutions, at the interface of a second pair of two interacting CH3 antibody constant domains, such that the first pair of two Fc domain monomers of an Fc domain selectively form a dimer with each other and the second pair of two Fc domain monomers of an Fc domain selectively form a dimer with each other, thus preventing the formation of unwanted multimers or aggregates.

The Fc-antigen binding domain constructs can be assembled in many ways. The Fc-antigen binding domain constructs can be assembled from asymmetrical tandem Fc domains. The Fc-antigen binding domain constructs can be assembled from singly branched Fc domains, where the branch point is at the N-terminal Fc domain. The Fc-antigen binding domain constructs can be assembled from singly branched Fc domains, where the branch point is at the C-terminal Fc domain. The Fc-antigen binding domain constructs can be assembled from singly branched Fc domains, where the branch point is neither at the N- or C-terminal Fc domain. The Fc-antigen binding domain constructs can be assembled to form bispecific constructs using long and short chains with different antigen binding domain sequences. The Fc-antigen binding domain constructs can be assembled to form bispecific and trispecific constructs using chains with different sets of heterodimerization mutations and different antigen binding domains. A bispecific Fc-antigen binding domain construct includes two different antigen binding domains. A trispecific Fc-antigen binding domain construct includes three different antigen binding domains.

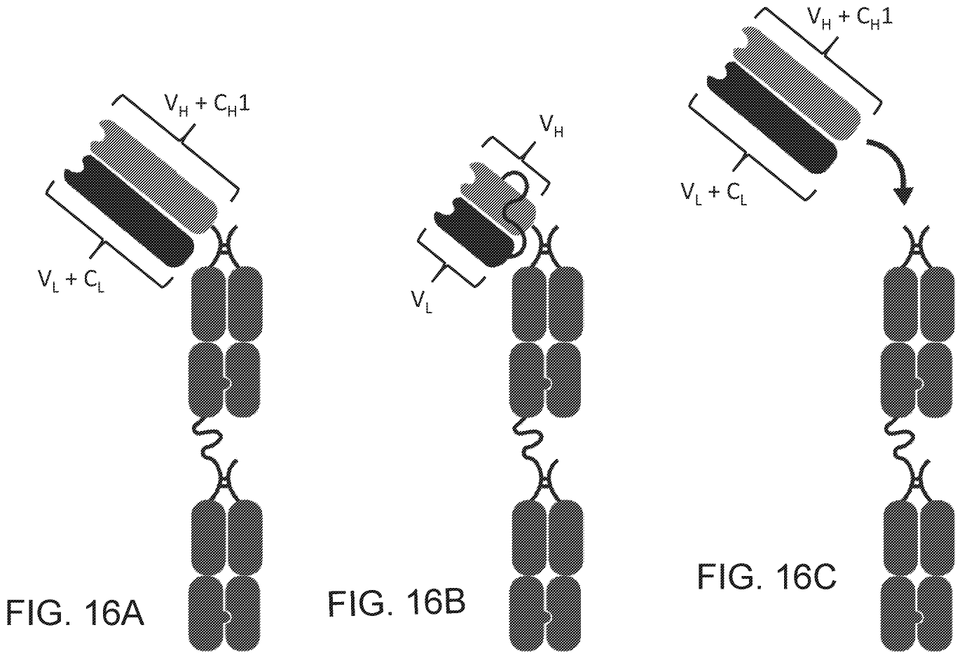

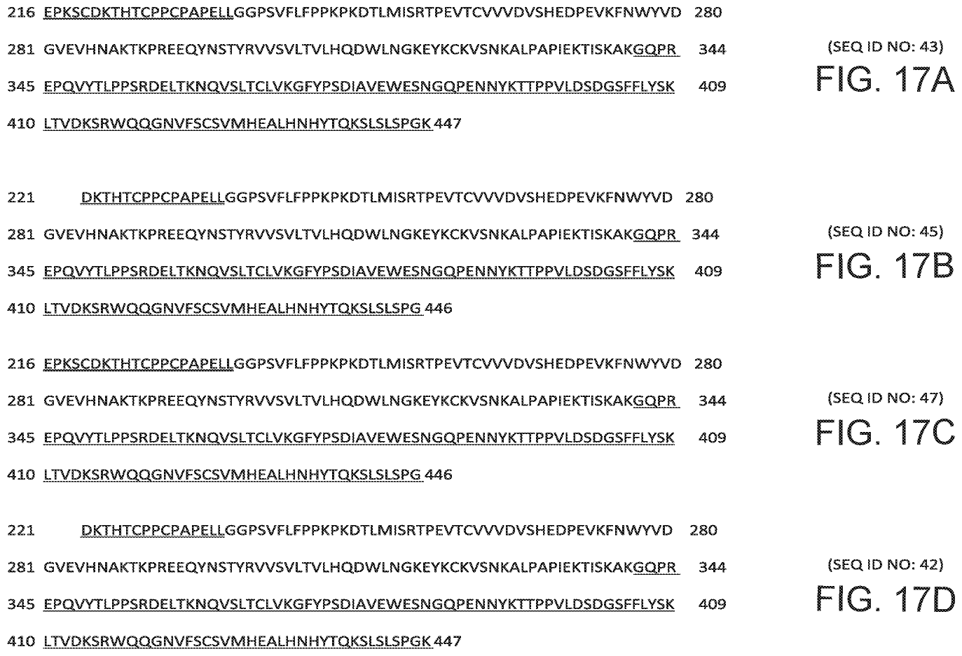

The antigen binding domain can be joined to the Fc-antigen binding domain construct in many ways. The antigen binding domain can be expressed as a fusion protein of an Fc chain. The heavy chain component of the antigen can be expressed as a fusion protein of an Fc chain and the light chain component can be expressed as a separate polypeptide (FIG. 16A). In some embodiments, a scFv is used as an antigen binding domain. The scFv can be expressed as a fusion protein of the long Fc chain (FIG. 16B). In some embodiments the heavy chain and light chain components are expressed separately and exogenously added to the Fc-antigen binding domain construct. In some embodiments, the antigen binding domain is expressed separately and later joined to the Fc-antigen binding domain construct with a chemical bond (FIG. 16C).

In some embodiments, one or more Fc polypeptides in an Fc-antigen binding domain construct lack a C-terminal lysine residue. In some embodiments, all of the Fc polypeptides in an Fc-antigen binding domain construct lack a C-terminal lysine residue. In some embodiments, the absence of a C-terminal lysine in one or more Fc polypeptides in an Fc-antigen binding domain construct may improve the homogeneity of a population of an Fc-antigen binding domain construct (e.g., an Fc-antigen binding domain construct having three Fc domains), e.g., a population of an Fc-antigen binding domain construct having three Fc domains that is at least 85%, 90%, 95%, 98%, or 99% homogeneous.

In some embodiments, the N-terminal Asp in an Fc-antigen binding domain construct described herein is mutated to Gln.

For the exemplary Fc-antigen binding domain constructs described in the Examples herein, Fc-antigen binding domain constructs 1-28 may contain the E357K and K370D charge pairs in the Knobs and Holes subunits, respectively. Fc-antigen binding domain constructs 29-42 can use orthogonal electrostatic steering mutations that may contain E357K and K370D pairings, and also could include additional steering mutations. For Fc-antigen binding constructs 29-42 with orthogonal knobs and holes electrostatic steering mutations are required all but one of the orthogonal pairs, and may be included in all of the orthogonal pairs.

In some embodiments, if two orthogonal knobs and holes are required, the electrostatic steering modification for Knob1 may be E357K and the electrostatic steering modification for Hole1 may be K370D, and the electrostatic steering modification for Knob2 may be K370D and the electrostatic steering modification for Hole2 may be E357K. If a third orthogonal knob and hole is needed (e.g. for a tri-specific antibody) electrostatic steering modifications E357K and D399K may be added for Knob3 and electrostatic steering modifications K370D and K409D may be added for Hole3 or electrostatic steering modifications K370D and K409D may be added for Knob3 and electrostatic steering modifications E357K and D399K may be added for Hole3.

Any one of the exemplary Fc-antigen binding domain constructs described herein (e.g. Fc-antigen binding domain constructs 1-42) can have enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain, or can include a biological activity that is not exhibited by a construct having a single Fc domain and the antigen binding domain.

### IX. Host Cells and Protein Production

In the present disclosure, a host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (transformation, transfection, electroporation, calcium phosphate precipitation, direct microinjection, etc.). Host cells can be of mammalian, bacterial, fungal or insect origin. Mammalian host cells include, but are not limited to, CHO (or CHO-derived cell strains, e.g., CHO-K1, CHO-DXB11 CHO-DG44), murine host cells (e.g., NS0, Sp2/0), VERY, HEK (e.g., HEK293), BHK, HeLa, COS, MDCK, 293, 3T3, W138, BT483, Hs578T, HTB2, BT20 and T47D, CRL7O3O and HsS78Bst cells. Host cells can also be chosen that modulate the expression of the protein constructs, or modify and process the protein product in the specific fashion desired. Different host cells have characteristic and specific mechanisms for the post-translational processing and modification of protein products. Appropriate cell lines or host systems can be chosen to ensure the correct modification and processing of the protein expressed.

For expression and secretion of protein products from their corresponding DNA plasmid constructs, host cells may be transfected or transformed with DNA controlled by appropriate expression control elements known in the art, including promoter, enhancer, sequences, transcription terminators, polyadenylation sites, and selectable markers. Methods for expression of therapeutic proteins are known in the art. See, for example, Paulina Balbas, Argelia Lorence (eds.) Recombinant Gene Expression: Reviews and Protocols (Methods in Molecular Biology), Humana Press; 2nd ed. 2004 edition (Jul. 20, 2004); Vladimir Voynov and Justin A. Caravella (eds.) Therapeutic Proteins: Methods and Protocols (Methods in Molecular Biology) Humana Press; 2nd ed. 2012 edition (Jun. 28, 2012).

In some embodiments, at least 50% of the Fc-antigen binding domain constructs that are produced by a host cell transfected with DNA plasmid constructs encoding the polypeptides that assemble into the Fc construct, e.g., in the cell culture supernatant, are structurally identical (on a molar basis), e.g., 50%, 60%, 70%, 80%, 90%, 95%, 100% of the Fc constructs are structurally identical.

### X. Afucosylation

Each Fc monomer includes an N-glycosylation site at Asn 297. The glycan can be present in a number of different forms on a given Fc monomer. In a composition containing antibodies or the antigen-binding Fc constructs described herein, the glycans can be quite heterogeneous and the nature of the glycan present can depend on, among other things, the type of cells used to produce the antibodies or antigen-binding Fc constructs, the growth conditions for the cells (including the growth media) and post-production purification. In various instances, compositions containing a construct described herein are afucosylated to at least some extent. For example, at least 5%, 10%, 15%, 20%, 25%, 30%, 35%, 40%, 45%, 50%, 60%, 70%, 80%, 90% or 95% of the glycans (e.g., the Fc glycans) present in the composition lack a fucose residue. Thus, 5%-60%, 5%-50%, 5%-40%, 10%-50%, 10%-50%, 10%-40%, 20%-50%, or 20%-40% of the glycans lack a fucose residue. Compositions that are afucosylated to at least some extent can be produced by culturing cells producing the antibody in the presence of 1,3,4-Tri-O-acetyl-2-deoxy-2-fluoro-L-fucose inhibitor. Relatively afucosylated forms of the constructs and polypeptides described herein can be produced using a variety of other methods, including: expressing in cells with reduced or no expression of FUT8 and expressing in cells that overexpress beta-1,4-mannosyl-glycoprotein 4-beta-N-acetylglucosaminyltransferase (GnT-III).

### XI. Purification

An Fc-antigen binding domain construct can be purified by any method known in the art of protein purification, for example, by chromatography (e.g., ion exchange, affinity (e.g., Protein A affinity), and size-exclusion column chromatography), centrifugation, differential solubility, or by any other standard technique for the purification of proteins. For example, an Fc-antigen binding domain construct can be isolated and purified by appropriately selecting and combining affinity columns such as Protein A column with chromatography columns, filtration, ultra filtration, salting-out and dialysis procedures (see, e.g., Process Scale Purification of Antibodies, Uwe Gottschalk (ed.) John Wiley & Sons, Inc., 2009; and Subramanian (ed.) Antibodies-Volume I-Production and Purification, Kluwer Academic/Plenum Publishers, New York (2004)).

In some instances, an Fc-antigen binding domain construct can be conjugated to one or more purification peptides to facilitate purification and isolation of the Fc-antigen binding domain construct from, e.g., a whole cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct include, but are not limited to, a hexa-histidine peptide (SEQ ID NO: 38), a FLAG peptide, a myc peptide, and a hemagglutinin (HA) peptide. A hexa-histidine peptide (SEQ ID NO: 38) (HHHHHH (SEQ ID NO: 38)) binds to nickel-functionalized agarose affinity column with micromolar affinity. In some embodiments, a FLAG peptide includes the sequence DYKDDDDK (SEQ ID NO: 39). In some embodiments, a FLAG peptide includes integer multiples of the sequence DYKDDDDK (SEQ ID NO: 39) in tandem series, e.g., 3×DYKDDDDK (SEQ ID NO: 318). In some embodiments, a myc peptide includes the sequence EQKLISEEDL (SEQ ID NO: 40). In some embodiments, a myc peptide includes integer multiples of the sequence EQKLISEEDL (SEQ ID NO: 40) in tandem series, e.g., 3×EQKLISEEDL (SEQ ID NO: 319). In some embodiments, an HA peptide includes the sequence YPYDVPDYA (SEQ ID NO: 41). In some embodiments, an HA peptide includes integer multiples of the sequence YPYDVPDYA (SEQ ID NO: 41) in tandem series, e.g., 3×YPYDVPDYA (SEQ ID NO: 320). Antibodies that specifically recognize and bind to the FLAG, myc, or HA purification peptide are well-known in the art and often commercially available. A solid support (e.g., a matrix, a resin, or agarose beads) functionalized with these antibodies may be used to purify an Fc-antigen binding domain construct that includes a FLAG, myc, or HA peptide.

For the Fc-antigen binding domain constructs, Protein A column chromatography may be employed as a purification process. Protein A ligands interact with Fc-antigen binding domain constructs through the Fc region, making Protein A chromatography a highly selective capture process that is able to remove most of the host cell proteins. In the present disclosure, Fc-antigen binding domain constructs may be purified using Protein A column chromatography as described in Examples 4-8. In some embodiments, use of the heterodimerizing and/or homodimerizing domains described herein allow for the preparation of an Fc-antigen binding domain construct with 60% or more purity, i.e., wherein 60% or more of the protein construct material produced in cells is of the desired Fc construct structure, e.g., 60%, 65%, 70%, 75%, 80%, 85%, 90%, 95%, 96%, 97%, 98%, 99%, or 100% of the protein construct material in a preparation is of the desired Fc construct structure. In some embodiments, less than 30% of the protein construct material in a preparation of an Fc-antigen binding domain construct is of an undesired Fc construct structure (e.g., a higher order species of the construct, as described in Example 1), e.g., 30%, 25%, 20%, 15%, 10%, 5%, 4%, 3%, 2%, 1%, or less of the protein construct material in a preparation is of an undesired Fc construct structure. In some embodiments, the final purity of an Fc-antigen binding domain construct, after further purification using one or more known methods of purification (e.g., Protein A affinity purification), can be 80% or more, i.e., wherein 80% or more of the purified protein construct material is of the desired Fc construct structure, e.g., 80%, 85%, 90%, 95%, 96%, 97%, 98%, 99%, or 100% of the protein construct material in a preparation is of the desired Fc construct structure. In some embodiments, less than 15% of protein construct material in a preparation of an Fc-antigen binding domain construct that is further purified using one or more known methods of purification (e.g., Protein A affinity purification) is of an undesired Fc construct structure (e.g., a higher order species of the construct, as described in Example 1), e.g., 15%, 10%, 5%, 4%, 3%, 2%, 1%, or less of the protein construct material in the preparation is of an undesired Fc construct structure.

### XII. Pharmaceutical Compositions/Preparations

The disclosure features pharmaceutical compositions that include one or more Fc-antigen binding domain constructs described herein. In one embodiment, a pharmaceutical composition includes a substantially homogenous population of Fc-antigen binding domain constructs that are identical or substantially identical in structure. In various examples, the pharmaceutical composition includes a substantially homogenous population of any one of Fc-antigen binding domain constructs 1-42.

A therapeutic protein construct, e.g., an Fc-antigen binding domain construct described herein (e.g., an Fc-antigen binding domain construct having three Fc domains), of the present disclosure can be incorporated into a pharmaceutical composition. Pharmaceutical compositions including therapeutic proteins can be formulated by methods know to those skilled in the art. The pharmaceutical composition can be administered parenterally in the form of an injectable formulation including a sterile solution or suspension in water or another pharmaceutically acceptable liquid. For example, the pharmaceutical composition can be formulated by suitably combining the Fc-antigen binding domain construct with pharmaceutically acceptable vehicles or media, such as sterile water for injection (WFI), physiological saline, emulsifier, suspension agent, surfactant, stabilizer, diluent, binder, excipient, followed by mixing in a unit dose form required for generally accepted pharmaceutical practices. The amount of active ingredient included in the pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50, and the like commonly known in the art. Formulation methods for therapeutic protein products are known in the art, see e.g., Banga (ed.) Therapeutic Peptides and Proteins: Formulation, Processing and Delivery Systems (2d ed.) Taylor & Francis Group, CRC Press (2006).

### XIII. Methods of Treatment and Dosage

The Fc antigen binding domain constructs described here in can be used to treat a variety of cancers (e.g., hematologic malignancies and solid tumors) and autoimmune diseases.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, oral dosage forms such as ingestible solutions, drug release capsules, and the like. The appropriate dosage for the individual subject depends on the therapeutic objectives, the route of administration, and the condition of the patient. Generally, recombinant proteins are dosed at 1-200 mg/kg, e.g., 1-100 mg/kg, e.g., 20-100 mg/kg. Accordingly, it will be necessary for a healthcare provider to tailor and titer the dosage and modify the route of administration as required to obtain the optimal therapeutic effect.

### XIV. Complement-Dependent Cytotoxicity (CDC)

Fc-antigen binding domain constructs described in this disclosure are able to activate various Fc receptor mediated effector functions. One component of the immune system is the complement-dependent cytotoxicity (CDC) system, a part of the innate immune system that enhances the ability of antibodies and phagocytic cells to clear foreign pathogens. Three biochemical pathways activate the complement system: the classical complement pathway, the alternative complement pathway, and the lectin pathway, all of which entail a set of complex activation and signaling cascades.

In the classical complement pathway, IgG or IgM trigger complement activation. The C1q protein binds to these antibodies after they have bound an antigen, forming the C1 complex. This complex generates C1s esterase, which cleaves and activates the C4 and C2 proteins into C4a and C4b, and C2a and C2b. The C2a and C4b fragments then form a protein complex called C3 convertase, which cleaves C3 into C3a and C3b, leading to a signal amplification and formation of the membrane attack complex.

The Fc-antigen binding domain constructs of this disclosure are able to enhance CDC activity by the immune system.

CDC may be evaluated by using a colorimetric assay in which antigen-expressing cells (e.g., Raji cells (ATCC)) are coated with a serially diluted antibody, Fc-antigen binding domain construct, or IVIg. Human serum complement (Quidel) can be added to all wells at 25% v/v and incubated for 2 h at 37° C. Cells can be incubated for 12 h at 37° C. after addition of WST-1 cell proliferation reagent (Roche Applied Science). Plates can then be placed on a shaker for 2 min and absorbance at 450 nm can be measured.

### XV. Antibody-Dependent Cell-Mediated Cytotoxicity (ADCC)

The Fc-antigen binding domain constructs of this disclosure are also able to enhance antibody-dependent cell-mediated cytotoxicity (ADCC) activity by the immune system. ADCC is a part of the adaptive immune system where antibodies bind surface antigens of foreign pathogens and target them for death. ADCC involves activation of natural killer (NK) cells by antibodies. NK cells express Fc receptors, which bind to Fc portions of antibodies such as IgG and IgM. When the antibodies are bound to the surface of a pathogen-infected target cell, they then subsequently bind the NK cells and activate them. The NK cells release cytokines such as IFN-γ, and proteins such as perforin and granzymes. Perforin is a pore forming cytolysin that oligomerizes in the presence of calcium. Granzymes are serine proteases that induce programmed cell death in target cells. In addition to NK cells, macrophages, neutrophils and eosinophils can also mediate ADCC.

ADCC may be evaluated using a luminescence assay. Human primary NK effector cells (Hemacare) are thawed and rested overnight at 37° C. in lymphocyte growth medium-3 (Lonza) at 5×10⁵/mL. The next day, the human lymphoblastoid cell line Raji target cells (ATCC CCL-86) are harvested, resuspended in assay media (phenol red free RPMI, 10% FBSΔ, GlutaMAX™), and plated in the presence of various concentrations of each probe of interest for 30 minutes at 37° C. The rested NK cells are then harvested, resuspended in assay media, and added to the plates containing the anti-CD20 coated Raji cells. The plates are incubated at 37° C. for 6 hours with the final ratio of effector-to-target cells at 5:1 (5×10⁴ NK cells: 1×10⁴ Raji).

The CytoTox-Glo™ Cytotoxicity Assay kit (Promega) is used to determined ADCC activity. The CytoTox-Glo™ assay uses a luminogenic peptide substrate to measure dead cell protease activity which is released by cells that have lost membrane integrity e.g. lysed Raji cells. After the 6 hour incubation period, the prepared reagent (substrate) is added to each well of the plate and placed on an orbital plate shaker for 15 minutes at room temperature. Luminescence is measured using the PHERAstar F5 plate reader (BMG Labtech). The data is analyzed after the readings from the control conditions (NK cells+Raji only) are subtracted from the test conditions to eliminate background.

### XVI. Antibody-Dependent Cellular Phagocytosis (ADCP)

The Fc-antigen binding domain constructs of this disclosure are also able to enhance antibody-dependent cellular phagocytosis (ADCP) activity by the immune system. ADCP, also known as antibody opsonization, is the process by which a pathogen is marked for ingestion and elimination by a phagocyte. Phagocytes are cells that protect the body by ingesting harmful foreign pathogens and dead or dying cells. The process is activated by pathogen-associated molecular patterns (PAMPS), which leads to NF-κB activation. Opsonins such as C3b and antibodies can then attach to target pathogens. When a target is coated in opsonin, the Fc domains attract phagocytes via their Fc receptors. The phagocytes then engulf the cells, and the phagosome of ingested material is fused with the lysosome. The subsequent phagolysosome then proteolytically digests the cellular material.

ADCP may be evaluated using a bioluminescence assay. Antibody-dependent cell-mediated phagocytosis (ADCP) is an important mechanism of action of therapeutic antibodies. ADCP can be mediated by monocytes, macrophages, neutrophils and dendritic cells via FcγRIIa (CD32a), FcγRI (CD64), and FcγRIIIa (CD16a). All three receptors can participate in antibody recognition, immune receptor clustering, and signaling events that result in ADCP; however, blocking studies suggest that FcγRIIa is the predominant Fcγ receptor involved in this process.

The FcγRIIa-H ADCP Reporter Bioassay is a bioluminescent cell-based assay that can be used to measure the potency and stability of antibodies and other biologics with Fc domains that specifically bind and activate FcγRIIa. The assay consists of a genetically engineered Jurkat T cell line that expresses the high-affinity human FcγRIIa-H variant that contains a Histidine (H) at amino acid 131 and a luciferase reporter driven by an NFAT-response element (NFAT-RE).

When co-cultured with a target cell and relevant antibody, the FcγRIIa-H effector cells bind the Fc domain of the antibody, resulting in FcγRIIa signaling and NFAT-RE-mediated luciferase activity. The bioluminescent signal is detected and quantified with a Luciferase assay and a standard luminometer.

### EXAMPLES

The following examples are put forth so as to provide those of ordinary skill in the art with a complete disclosure and description of how the methods and compounds claimed herein are performed, made, and evaluated, and are intended to be purely exemplary of the disclosure and are not intended to limit the scope of what the inventors regard as their disclosure.

### Example 1. Use of Orthogonal Heterodimerizing Domains to Control the Assembly of Linear Fc-Antigen Domain Containing Polypeptides

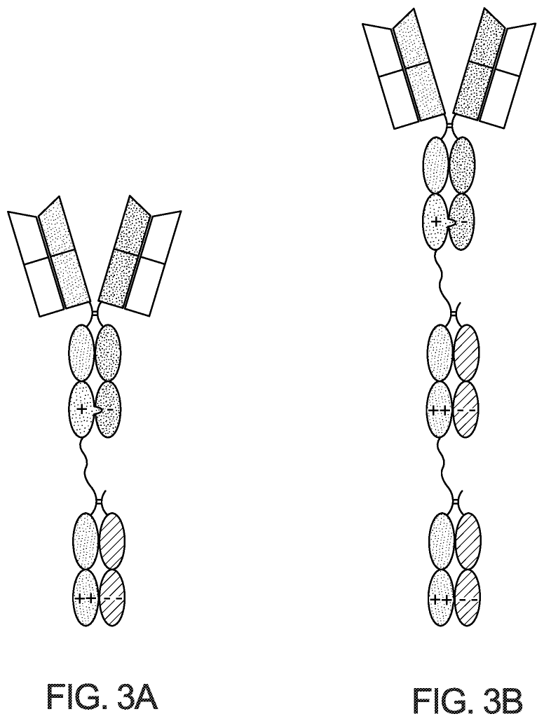

A variety of approaches to appending Fc domains to the C-termini of antibodies have been described, including in the production of tandem Fc constructs with and without peptide linkers between Fc domains (see, e.g., Nagashima et al., Mol Immunol, 45:2752-63, 2008, and Wang et al. MAbs, 9:393-403, 2017). However, methods described in the scientific literature for making antibody constructs with multiple Fc domains are limited in their effectiveness because these methods result in the production of numerous undesired species of Fc domain containing proteins. These species have different molecular weights that result from uncontrolled off-register association of polypeptide chains during product production, resulting in a ladder of molecular weights (see, e.g., Nagashima et al., Mol Immunol, 45:2752-63, 2008, and Wang et al. MAbs, 9:393-403, 2017). FIG. 1 and FIG. 2 schematically depict some examples of the protein species with multiple Fc domains of various molecular weights that can be produced by the off register association of polypeptides containing two tandem Fc monomers (FIG. 1) or three tandem Fc monomers (FIG. 3). Consistently achieving a desired Fc-antigen binding domain construct with multiple Fc domains having a defined molecular weight using these existing approaches requires the removal of higher order species (HOS) with larger molecular weights, which greatly reduces the yield of the desired construct.

The use of orthogonal heterodimerization domains allowed for the production of structures with tandem Fc extensions without also generating large amounts of higher order species (HOS). FIGS. 3A and 3B depict examples of orthogonal linear Fc-antigen domain binding constructs with two Fc domains (FIG. 3A) or 3 Fc domains (FIG. 3B) that are produced by joining one long polypeptide with multiple Fc domain monomers to two different short polypeptides, each with a single Fc monomer. In these examples, one Fc domain of each construct includes knobs-into-holes mutations in combination with a reverse charge mutation in the CH3-CH3 interface of the Fc domain, and two reverse charge mutations in the CH3-CH3 interface of either 1 other Fc domain (FIG. 3A) or 2 other Fc domains (FIG. 3B). Short polypeptide chains with Fc monomers having the two reverse charge mutations have a lower affinity for the long chain Fc monomer having protuberance-forming mutations and a single reverse charge mutation, and are much more likely to bind to the long chain Fc monomer(s) having 2 compatible reverse charge mutations. The short polypeptide chains with Fc monomers having cavity-forming mutations in combination with a reverse charge mutation are much more likely to bind to the long chain Fc monomer having protuberance-forming mutations in combination with a compatible reverse charge mutation.

### Example 2. Types of Fc Construct Structures that can be Generated Using Orthogonal Heterodimerizing Domains

Orthogonal heterodimerization domains having different knob-into-hole and/or electrostatic reverse charge mutations selected from Tables 3 and 4 can be integrated into different polypeptide chains to control the positioning of multiple antigen binding domains and Fc domains during assembly of Fc-antigen binding domain constructs. A large variety of Fc-antigen binding domain constructs of varying structures can be generated using design principles that incorporate at least two orthogonal heterodimerization domains into the polypeptide chains that assemble into the constructs.

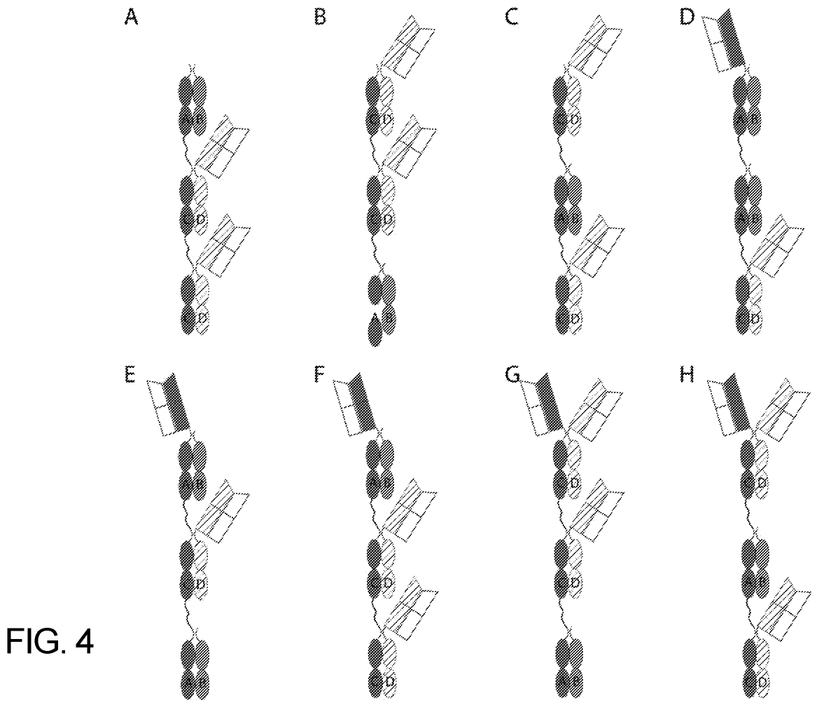

FIG. 4 depicts some examples of linear tandem Fc constructs that are assembled using orthogonal heterodimerization technologies. These structural examples demonstrate the use of two different sets of heterodimerizing mutations (a first set of heterodimerization mutations in the Fc monomers of one group of Fc domains (A and B) and a second set of heterodimerization mutations in the Fc monomers of another group of Fc domains (C and D)) to control the positioning of multiple antigen binding domains at various particular locations along a construct with three tandem Fc domains. Examples 4, 5, and 6 describe the production of orthogonal linear Fc-antigen domain binding constructs that correspond to the structures depicted in the schematics of FIGS. 4A, 4B, and 4D. Constructs 45, 46, and 47, having either anti-CD20 or anti-PD-L1 domains, were produced with minimal undesired higher order species, and tested for functionality using CDC, ADCP, and ADCC assays.

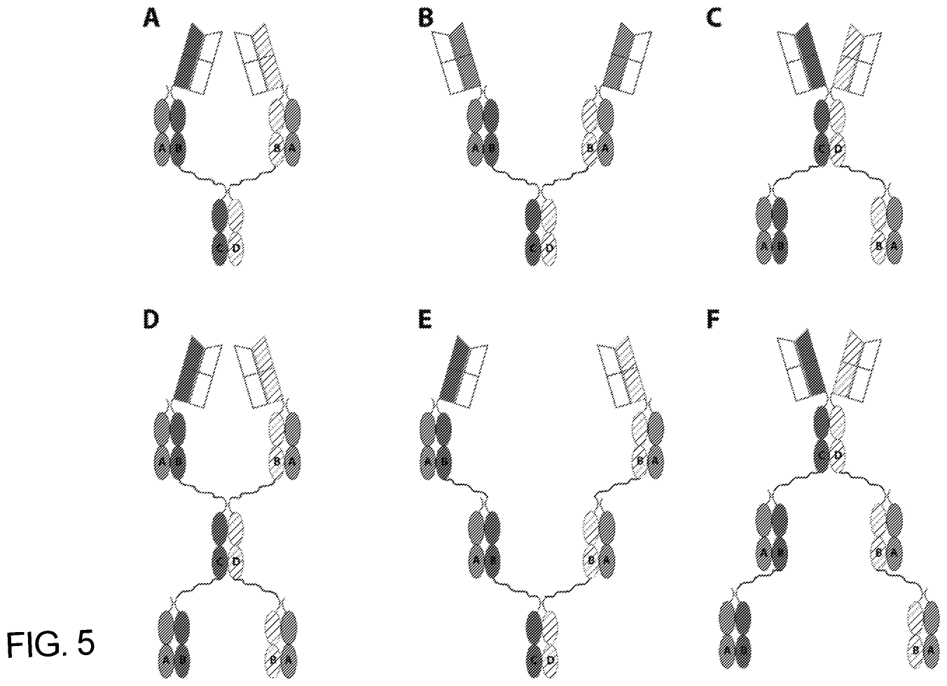

Orthogonal heterodimerization technologies can also be used to produce branched Fc-antigen binding domain constructs that have a symmetrical distribution of antigen-binding domains and Fc domains using an asymmetrical arrangement of polypeptide chains. FIG. 5 depicts some examples of these Fc constructs. The constructs have two long polypeptide chains joined together at one Fc domain using a set of heterodimerization mutations (the C and D heterodimerization pair). Another set of heterodimerization mutations (the A and B heterodimerization pair) promotes the association of additional Fc domain monomers of the long chain polypeptide with a compatible Fc domain monomer on a small chain polypeptide. These branched constructs are structurally similar to the symmetrical branched constructs than can be produced using a single homodimerized Fc domain.

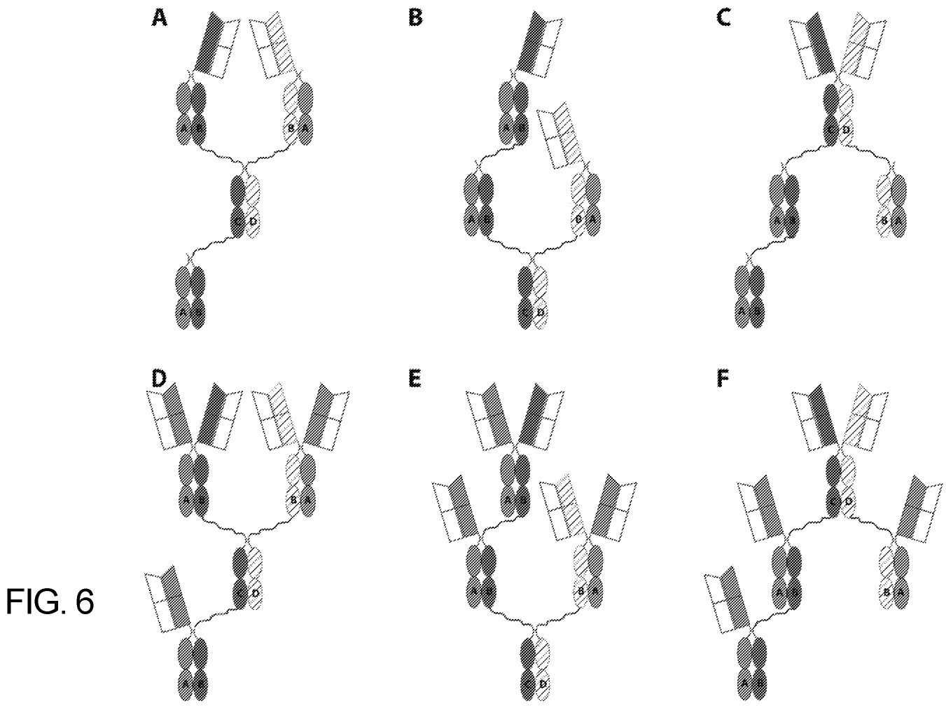

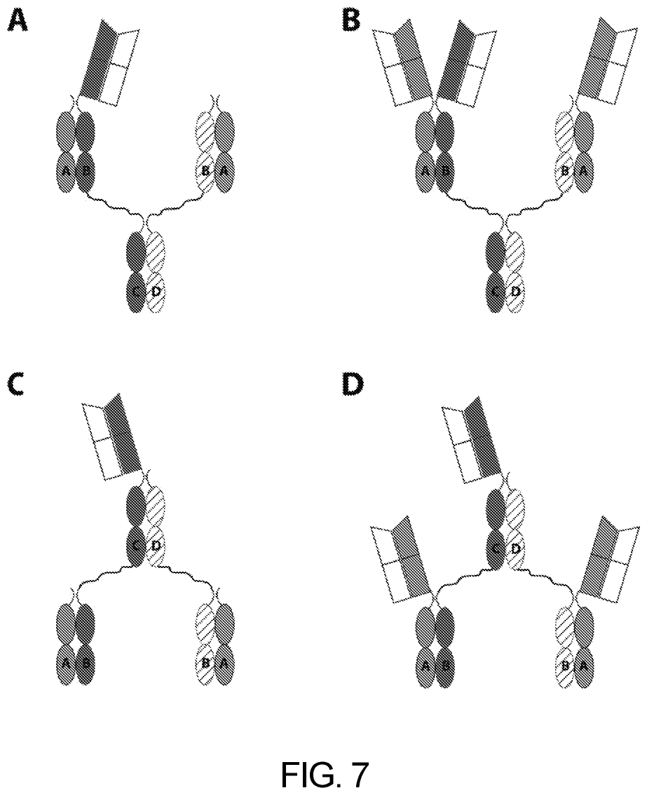

Asymmetrically branched Fc-antigen binding domain constructs can also be produced using orthogonal heterodimerization technologies. FIG. 6 depicts some examples of asymmetrically branched Fc constructs. The constructs are produced by joining two polypeptide chains of different length that have a different number of Fc domains (e.g., polypeptide chains with 3 Fc domains and 2 Fc domains) at one Fc domain using a one set of heterodimerizing mutations (the C and D heterodimerization pair). A different set of heterodimerization mutations (the A and B heterodimerization pair) promotes the association of additional Fc domain monomers on these polypeptide chains with a compatible Fc domain monomer on a small chain polypeptide. Alternatively, FIG. 7 depicts examples of asymmetrically branched Fc constructs produced by joining two long polypeptide chains (having an equal number of Fc domains) at one Fc domain using a one set of heterodimerizing mutations (the C and D heterodimerization pair), with an odd number of antigen binding domains distributed asymmetrically on the molecule.

### Example 3. Preparation of Asymmetrically Branched Fc-Antigen Binding Domain Constructs

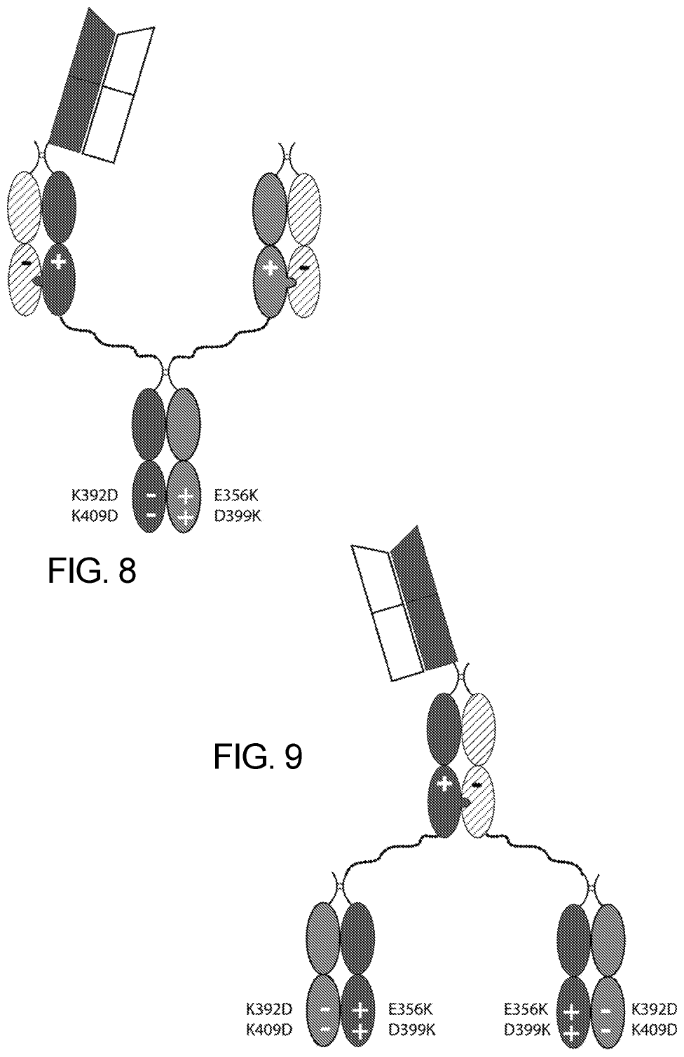

Two different Fc-containing constructs were designed and produced in cells to test whether asymmetrically branched Fc-antigen binding domain constructs could be effectively produced using orthogonal heterodimerizing technologies. The two Fc constructs (FIG. 8 and FIG. 0) each had three Fc domains and were assembled from three different polypeptides using two sets of heterodimerization domain mutations. Both constructs were branched Fc constructs with a symmetrical distribution of Fc domains using an asymmetrical arrangement of polypeptide chains, and each had a single anti-CD20 Fab domain that was asymmetrically distributed on the construct. FIG. 8 depicts an Fc construct with three Fc domains, wherein two of the Fc domains had knobs-into-holes mutations in combination with an electrostatic steering mutation (one Fc monomer having S354C and T366W protuberance-forming mutations and a E357K reverse charge mutation and the other Fc monomer having Y349C, T366S, L368A, and Y407V cavity-forming mutations in combination with a K370D reverse charge mutation), and one of the Fc domains had electrostatic steering mutations (one Fc monomer having D356K and D399K reverse charge mutations and the other Fc monomer having K392D and K409D reverse charge mutations). FIG. 9 depicts an Fc construct with an inverse structure relative to the structure of FIG. 8, that is assembled using the same heterodimerizing mutations, except that the FIG. 9 Fc structure had one Fc domain with knobs-into-holes mutations in combination with an electrostatic steering mutation and two Fc domains with only electrostatic steering mutations. Table 8 depicts the sequences for these constructs.

TABLE 8
Sequences for the constructs depicted in FIGs. 8 and 9

| | | | Second | |
|---|---|---|---|---|
| | | Long Fc | Long Fc | |
| | | chain | chain | |
| | | (with | (no | |
| | | anti-CD20 | anti-CD20 | |
| | | VH and | VH and | Short Fc |
| Construct | Light chain | CH1) | CH1) | chain |
| FIG. 8 | SEQ ID | SEQ ID | SEQ ID | SEQ ID |
| construct | NO: 61 | NO: 321 | NO: 322 | NO: 48 |
| (CD20) | DIVMTQTPLSL | QVQLVQSGAEVK | DKTHTCPPCPA | DKTHTCPPCPA |
| | PVTPGEPASIS | KPGSSVKVSCKA | PELLGGPSVFL | PELLGGPSVFL |
| | CRSSKSLLHSN | SGYAFSYSWINW | FPPKPKDTLMI | FPPKPKDTLMI |
| | GITYLYWYLQK | VRQAPGQGLEWM | SRTPEVTCVVV | SRTPEVTCVVV |
| | PGQSPQLLIYQ | GRIFPGDGDTDY | DVSHEDPEVKF | DVSHEDPEVKF |
| | MSNLVSGVPDR | NGKFKGRVTITA | NWYVDGVEVHN | NWYVDGVEVHN |
| | FSGSGSGTDFT | DKSTSTAYMELS | AKTKPREEQYN | AKTKPREEQYN |
| | LKISRVEAEDV | SLRSEDTAVYYC | STYRWSVLTVL | STYRVVSVLTV |
| | GVYYCAQNLEL | ARNVFDGYWLVY | HQDWLNGKEYK | LHQDWLNGKEY |
| | PYTFGGGTKVE | WGQGTLVTVSSA | CKVSNKALPAP | KCKVSNKALPA |
| | IKRTVAAPSVF | STKGPSVFPLAP | IEKTISKAKGQ | PIEKTISKAKG |
| | IFPPSDEQLKS | SSKSTSGGTAAL | PREPQVYTLPP | QPREPQVCTLP |
| | GTASVVCLLNN | GCLVKDYFPEPV | CRDKLTKNQVS | PSRDELTKNQV |
| | FYPREAKVQWK | TVSWNSGALTSG | LWCLVKGFYPS | SLSCAVDGFYP |
| | VDNALQSGNSQ | VHTFPAVLQSSG | DIAVEWESNGQ | SDIAVEWESNG |
| | ESVTEQDSKDS | LYSLSSVVTVPSS | PENNYKTTPPV | QPENNYKTTPP |
| | TYSLSSTLTLS | SLGTQTYICNVN | LDSDGSFFLYS | VLDSDGSFFLV |
| | KADYEKHKVYA | HKPSNTKVDKKV | KLTVDKSRWQQ | SKLTVDKSRWQ |
| | CEVTHQGLSSP | EPKSCDKTHTCP | GNVFSCSVMHE | QGNVFSCSVMH |
| | VTKSFNRGEC | PCPAPELLGGPS | ALHNHYTQKSL | EALHNHYTQKS |
| | | VFLFPPKPKDTL | SLSPGKGGGGG | LSLSPG |
| | | MISRTPEVTCVV | GGGGGGGGGGG | |
| | | VDVSHEDPEVKF | GGGGDKTHTCP | |
| | | NWYVDGVEVHNA | PCPAPELLGGP | |
| | | KTKPREEQYNST | SVFLFPPKPKD | |
| | | YRVVSVLTVLHQ | TLMISRTPEVT | |
| | | DWLNGKEYKCKV | CVVVDVSHEDP | |
| | | SNKALPAPIEKT | EVKFNWYVDGV | |
| | | ISKAKGQPREPQ | EVHNAKTKPRE | |
| | | VYTLPPCRDKLT | EQYNSTYRVVS | |
| | | KNQVSLWCLVKG | VLTVLHQDWLN | |
| | | FYPSDIAVEWES | GKEYKCKVSNK | |
| | | NGQPENNYKTTP | ALPAPIEKTIS | |
| | | PVLDSDGSFFLY | KAKGQPREPQV | |
| | | SKLTVDKSRWQQ | YTLPPSRDELT | |
| | | GNVFSCSVMHEA | KNQVSLTCLVK | |
| | | LHNHYTQKSLSL | GFYPSDIAVEW | |
| | | SPGKGGGGGGGG | ESNGQPENNYD | |
| | | GGGGGGGGGGGG | TTPPVLDSDGS | |
| | | DKTHTCPPCPAP | FFLYSDLTVDK | |
| | | ELLGGPSVFLFP | SRWQQGNVFSC | |
| | | PKPKDTLMISRT | SVMHEALHNHY | |
| | | PEVTCVVVDVSH | TQKSLSLSPG | |
| | | EDPEVKFNWYVD | | |
| | | GVEVHNAKTKPR | | |
| | | EEQYNSTYRVVS | | |
| | | VLTVLHQDWLNG | | |
| | | KEYKCKVSNKAL | | |
| | | PAPIEKTISKAK | | |
| | | GQPREPQVYTLP | | |
| | | PSRKELTKNQVS | | |
| | | LTCLVKGFYPSD | | |
| | | IAVEWESNGQPE | | |
| | | NNYKTTPPVLKS | | |
| | | DGSFFLYSKLTV | | |
| | | DKSRWQQGNVFS | | |
| | | CSVMHEALHNHY | | |
| | | TQKSLSLSPGQ | | |
| FIG. 9 | SEQ ID | SEQ ID | SEQ ID | SEQ ID |
| construct | NO: 61 | NO: 321 | NO: 323 | NO: 236 |
| (CD-20) | DIVMTQTPLSL | QVQLVQSGAEVK | DKTHTCPPCPAP | DKTHTCPPCP |
| | PVTPGEPASIS | KPGSSVKVSCKA | ELLGGPSVFLFP | APELLGGPSV |
| | CRSSKSLLHSN | SGYAFSYSWINW | PKPKDTLMISRT | FLFPPKPKDT |
| | GITYLYWYLQK | VRQAPGQGLEWM | PEVTCVVVDVSH | LMISRTPEVT |
| | PGQSPQLLIYQ | GRIFPGDGDTDY | EDPEVKFNWYVD | CVVVDVSHED |
| | MSNLVSGVPDR | NGKFKGRVTITA | GVEVHNAKTKPR | PEVKFNWYVD |
| | FSGSGSGTDFT | DKSTSTAYMELS | EEQYNSTYRWSV | GVEVHNAKTK |
| | LKISRVEAEDV | SLRSEDTAVYYC | LTVLHQDWLNGK | PREEQYNSTY |
| | GVYYCAQNLEL | ARNVFDGYWLVY | EYKCKVSNKALP | RVVSVLTVLH |
| | PYTFGGGTKVE | WGQGTLVTVSSA | APIEKTISKAKG | QDWLNGKEYK |
| | IKRTVAAPSVF | STKGPSVFPLAP | QPREPQVCTLPP | CKVSNKALPA |
| | IFPPSDEQLKS | SSKSTSGGTAAL | SRDELTKNQVSL | PIEKTISKAK |
| | GTASVVCLLNN | GCLVKDYFPEPV | SCAVDGFYPSDI | GQPREPQVYT |
| | FYPREAKVQWK | TVSWNSGALTSG | AVEWESNGQPEN | LPPSRDELTK |
| | VDNALQSGNSQ | VHTFPAVLQSSG | NYKTTPPVLDSD | NQVSLTCLVK |
| | ESVTEQDSKDS | LYSLSSVVTVPSS | GSFFLVSKLTVD | GFYPSDIAVE |
| | TYSLSSTLTLS | SLGTQTYICNVN | KSRWQQGNVFSC | WESNGQPENN |
| | KADYEKHKVYA | HKPSNTKVDKKV | SVMHEALHNHYT | YDTTPPVLDS |
| | CEVTHQGLSSP | EPKSCDKTHTCP | QKSLSLSPGKGG | DGSFFLYSDL |
| | VTKSFNRGEC | PCPAPELLGGPS | GGGGGGGGGGGG | TVDKSRWQQG |
| | | VFLFPPKPKDTL | GGGGGGDKTHTC | NVFSCSVMHE |
| | | MISRTPEVTCVV | PPCPAPELLGGP | ALHNHYTQKS |
| | | VDVSHEDPEVKF | SVFLFPPKPKDT | LSLSPG |
| | | NWYVDGVEVHNA | LMISRTPEVTCV | |
| | | KTKPREEQYNST | VVDVSHEDPEVK | |
| | | YRVVSVLTVLHQ | FNWYVDGVEVHN | |
| | | DWLNGKEYKCKV | AKTKPREEQYNS | |
| | | SNKALPAPIEKT | TYRVVSVLTVLH | |
| | | ISKAKGQPREPQ | QDWLNGKEYKCK | |
| | | VYTLPPCRDKLT | VSNKALPAPIEK | |
| | | KNQVSLWCLVKG | TISKAKGQPREP | |
| | | FYPSDIAVEWES | QVYTLPPSRKEL | |
| | | NGQPENNYKTTP | TKNQVSLTCLVK | |
| | | PVLDSDGSFFLY | GFYPSDIAVEWE | |
| | | SKLTVDKSRWQQ | SNGQPENNYKTT | |
| | | GNVFSCSVMHEA | PPVLKSDGSFFL | |
| | | LHNHYTQKSLSL | YSKLTVDKSRWQ | |
| | | SPGKGGGGGGGG | QGNVFSCSVMHE | |
| | | GGGGGGGGGGGG | ALHNHYTQKSLS | |
| | | DKTHTCPPCPAP | LSPGQ | |
| | | ELLGGPSVFLFP | | |
| | | PKPKDTLMISRT | | |
| | | PEVTCVVVDVSH | | |
| | | EDPEVKFNWYVD | | |
| | | GVEVHNAKTKPR | | |
| | | EEQYNSTYRVVS | | |
| | | VLTVLHQDWLNG | | |
| | | KEYKCKVSNKAL | | |
| | | PAPIEKTISKAK | | |
| | | GQPREPQVYTLP | | |
| | | PSRKELTKNQVS | | |
| | | LTCLVKGFYPSD | | |
| | | IAVEWESNGQPE | | |
| | | NNYKTTPPVLKS | | |
| | | DGSFFLYSKLTV | | |
| | | DKSRWQQGNVFS | | |
| | | CSVMHEALHNHY | | |
| | | TQKSLSLSPGQ | | |

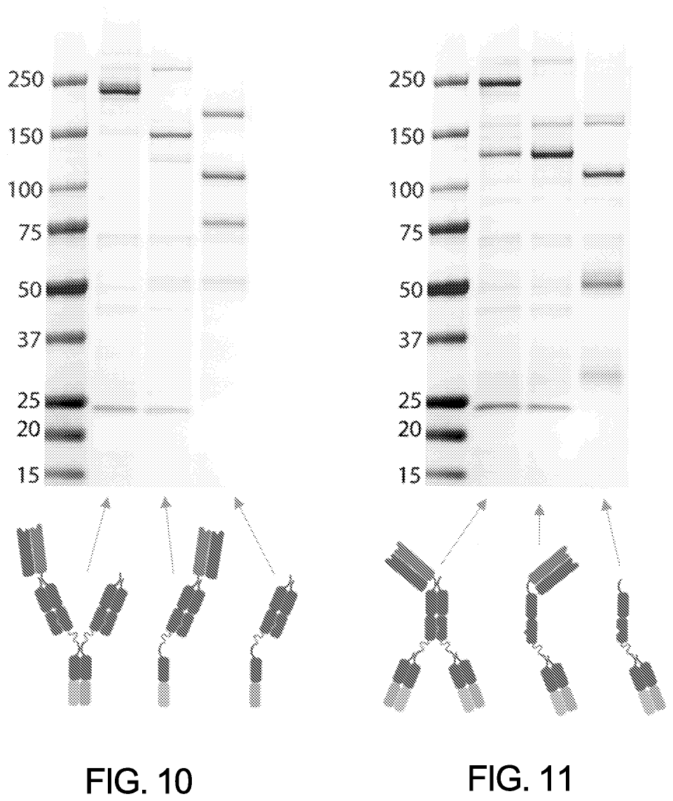

Each construct was expressed in HEK cells and the media was analyzed by SDS-PAGE. FIG. 10 shows that the predominant protein band for the construct depicted in FIG. 8 was at 200 kDa, as expected for the desired product. The only other combination of the four amino acid sequences used to produce this construct that could produce a 200 kDa product would be the combination of two copies of the Fab light chain with two copies of the long chain containing two Fc domains in tandem with the Fab VH and CH1 domains with failure of both heterodimerization mutants in the chain from self-associating. However, this self-association of heterodimerizing Fc sequences was not observed for the corresponding Fab-less construct (data not shown). Similarly, FIG. 11 shows that the predominant protein band for the construct depicted in FIG. 9 had a molecular weight that was slightly higher than 200 kDa, the expected weight for this product. The only other combination of the four amino acid sequences used to produce this construct that could produce a 200 kDa product would be the combination of two copies of the Fab light chain with two copies of the long chain containing two Fc domains in tandem with the Fab VH and CH1 domains with failure of both heterodimerization mutants in the chain from self-associating. However, this self-association of heterodimerizing Fc sequences was not observed for the corresponding Fab-less construct (data not shown).

### Example 4. Design and Purification of Fc-Antigen Binding Domain Construct 45 with an Anti-CD20 Antigen Binding Domain or an Anti-PD-L1 Antigen Binding Domain

Fc-antigen binding domain constructs are designed to increase folding efficiencies, to minimize uncontrolled association of subunits, which may create unwanted high molecular weight oligomers and multimers, and to generate compositions for pharmaceutical use that are substantially homogenous (e.g., at least 85%, 90%, 95%, 98%, or 99% homogeneous). With these goals in mind, an unbranched construct formed from tandem Fc domains (FIG. 12) was made as described below. Fc-antigen binding domain construct 45 (CD20) and construct 45 (PD-L1) each include three distinct Fc monomer containing polypeptides (either an anti-CD20 long Fc chain (SEQ ID NO: 239) or an anti-PD-L1 long Fc chain (SEQ ID NO: 240); a copy of a first short Fc chain that is an anti-CD20 short Fc chain (SEQ ID NO: 247) or an anti-PD-L1 Fc short chain (SEQ ID NO: 248); and two copies of a second short Fc chain (SEQ ID NO: 63)), and two copies of either an anti-CD20 light chain polypeptide (SEQ ID NO: 61) or an anti-PD-L1 light chain polypeptide (SEQ ID NO: 49), respectively. The long Fc chain contains three Fc domain monomers, each with a set of protuberance-forming mutations selected from Table 3 and/or one or more reverse charge mutation selected from Table 4, (the first Fc domain monomer with a different set of heterodimerization mutations than the second and third Fc domain monomers) in a tandem series with an antigen binding domain at the N-terminus. The first short Fc chain contains an Fc domain monomer with a first set of cavity-forming mutations selected from Table 3 and/or one or more reverse charge mutation selected from Table 4 (wherein the mutations are different from a second set of mutations in the second short Fc chain), and an antigen binding domain at the N-terminus. The second short Fc chain contains an Fc domain monomer with a second set of cavity-forming mutations selected from Table 3, and/or one or more reverse charge mutation selected from Table 4 (wherein the mutations are different from the first set off mutations in the first short Fc chain).

In this case, the long Fc chain contains one Fc domain monomer with D356K and D399K charge mutations in a tandem series with two Fc domain monomers with S354C and T366W protuberance-forming mutations and a E357K charge mutation, and either anti-CD20 VH and CH1 domains (EU positions 1-220) at the N-terminus (construct 45 (CD20) or anti-PD-L1 VH and CH1 domains (EU positions 1-220) at the N-terminus (construct 45 (PD-L1)). The first short Fc chain contains an Fc domain monomer with a K392D and K409D charge mutations, and either anti-CD20 VH and CH1 domains (EU positions 1-220) at the N-terminus (construct 45 (CD20)) or anti-PD-L1 VH and CH1 domains (EU positions 1-220) at the N-terminus (construct 45 (PD-L1)). The second short Fc chain contains an Fc domain monomer with Y349C, T366S, L368A, and Y407V cavity-forming mutations and a K370D charge mutation.

TABLE 9
Construct 45 (CD20) and Construct 45 (PD-L1) sequences

| | | | First | |
|---|---|---|---|---|
| | | Long Fc | Short | |
| | | chain | Fc chain | |
| | | (with | (with | |
| | | anti-CD20 | anti-CD20 | Second |
| | | or anti- | or anti- | Short |
| | | PD-L1 VH | PD-L1 VH | Fc |
| Construct | Light chain | and CH1) | and CH1) | chain |
| Construct | SEQ ID NO: 61 | SEQ ID NO: 239 | SEQ ID NO: 247 | SEQ ID NO: 63 |
| 45 (CD20) | DIVMTQTPLS | QVQLVQSGAEV | QVQLVQSGAE | DKTHTCPPCP |
| | LPVTPGEPAS | KKPGSSVKVSC | VKKPGSSVKV | APELLGGPSV |
| | ISCRSSKSLL | KASGYAFSYSW | SCKASGYAFS | FLFPPKPKDT |
| | HSNGITYLYW | INWVRQAPGQG | YSWINWVRQA | LMISRTPEVT |
| | YLQKPGQSPQ | LEWMGRIFPGD | PGQGLEWMGR | CVVVDVSHED |
| | LLIYQMSNLV | GDTDYNGKFKG | IFPGDGDTDY | PEVKFNWYVD |
| | SGVPDRFSGS | RVTITADKSTS | NGKFKGRVTI | GVEVHNAKTK |
| | GSGTDFTLKI | TAYMELSSLRS | TADKSTSTAY | PREEQYNSTY |
| | SRVEAEDVGV | EDTAVYYCARN | MELSSLRSED | RVVSVLTVLH |
| | YYCAQNLELP | VFDGYWLVYWG | TAVYYCARNV | QDWLNGKEYK |
| | YTFGGGTKVE | QGTLVTVSSAS | FDGYWLVYWG | CKVSNKALPA |
| | IKRTVAAPSV | TKGPSVFPLAP | QGTLVTVSSA | PIEKTISKAK |
| | FIFPPSDEQL | SSKSTSGGTAA | STKGPSVFPL | GQPREPQVCT |
| | KSGTASVVCL | LGCLVKDYFPE | APSSKSTSGG | LPPSRDELTK |
| | LNNFYPREAK | PVTVSWNSGAL | TAALGCLVKD | NQVSLSCAVD |
| | VQWKVDNALQ | TSGVHTFPAVL | YFPEPVTVSW | GFYPSDIAVE |
| | SGNSQESVTE | QSSGLYSLSSW | NSGALTSGVH | WESNGQPENN |
| | QDSKDSTYSL | TVPSSSLGTQT | TFPAVLQSSG | YKTTPPVLDS |
| | SSTLTLSKAD | YICNVNHKPSN | LYSLSSVVTVP | DGSFFLVSKL |
| | YEKHKVYACE | TKVDKKVEPKS | SSSLGTQTYI | TVDKSRWQQG |
| | VTHQGLSSPV | CDKTHTCPPCP | CNVNHKPSNT | NVFSCSVMHE |
| | TKSFNRGEC | APELLGGPSVF | KVDKKVEPKS | ALHNHYTQKS |
| | | LFPPKPKDTLM | CDKTHTCPPC | LSLSPG |
| | | ISRTPEVTCVV | PAPELLGGPS | |
| | | VDVSHE | VFLFPPKPKD | |
| | | DPEVKFNWYV | TLMISRTP | |
| | | DGVEVHNAKTK | EVTCVVVDV | |
| | | PREEQYNSTYR | SHEDPEVKF | |
| | | VVSVLTVLHQD | NWYVDGVEV | |
| | | WLNGKEYKCKV | HNAKTKPRE | |
| | | SNKALPAPIEK | EQYNSTYRV | |
| | | TISKAKGQPRE | VSVLTVLHQ | |
| | | PQVYTLPPCRD | DWLNGKEYK | |
| | | KLTKNQVSLWC | CKVSNKALP | |
| | | LVKGFYPSDIA | APIEKTISK | |
| | | VEWESNGQPEN | AKGQPREPQ | |
| | | NYKTTPPVLDS | VYTLPPSRD | |
| | | DGSFFLYSKLT | ELTKNQVSL | |
| | | VDKSRWQQGNV | TCLVKGFYP | |
| | | FSCSVMHEALH | SDIAVEWES | |
| | | NHYTQKSLSLS | NGQPENNYD | |
| | | PGKGGGGGGGG | TTPPVLDSD | |
| | | GGGGGGGGGGG | GSFFLYSDL | |
| | | GDKTHTCPPCP | TVDKSRWQQ | |
| | | APELLGGPSVF | GNVFSCSVM | |
| | | LFPPKPKDTLM | HEALHNHYT | |
| | | ISRTPEVTCVV | QKSLSLSPG | |
| | | VDVSHEDPEVK | | |
| | | FNWYVDGVEVH | | |
| | | NAKTKPREEQY | | |
| | | NSTYRVVSVLT | | |
| | | VLHQDWLNGKE | | |
| | | YKCKVSNKALP | | |
| | | APIEKTISKAK | | |
| | | GQPREPQVYTL | | |
| | | PPCRDKLTKNQ | | |
| | | VSLWCLVKGFY | | |
| | | PSDIAVEWESN | | |
| | | GQPENNYKTTP | | |
| | | PVLDSDGSFFL | | |
| | | YSKLTVDKSRW | | |
| | | QQGNVFSCSVM | | |
| | | HEALHNHYTQK | | |
| | | SLSLSPGKGGG | | |
| | | GGGGGGGGGGG | | |
| | | GGGGGGDKTHT | | |
| | | CPPCPAPELLG | | |
| | | GPSVFLFPPKP | | |
| | | KDTLMISRTPE | | |
| | | VTCVWDVSHED | | |
| | | PEVKFNWYVDG | | |
| | | VEVHNAKTKPR | | |
| | | EEQYNSTYRVV | | |
| | | SVLTVLHQDWL | | |
| | | NGKEYKCKVSN | | |
| | | KALPAPIEKTI | | |
| | | SKAKGQPREPQ | | |
| | | VYTLPPSRKEL | | |
| | | TKNQVSLTCLV | | |
| | | KGFYPSDIAVE | | |
| | | WESNGQPENNY | | |
| | | KTTPPVLKSDG | | |
| | | SFFLYSKLTVD | | |
| | | KSRWQQGNVFS | | |
| | | CSVMHEALHNH | | |
| | | YTQKSLSLSPG | | |
| | | Q | | |
| Construct | SEQ ID NO: 49 | SEQ ID NO: 240 | SEQ ID NO: 248 | SEQ ID NO: 63 |
| 45 (PD-L1) | | EVQLLESGGG | EVQLLESGGG | DKTHTCPPCP |
| | QSALTQPASVS | LVQPGGSLRL | LVQPGGSLRLS | APELLGGPSV |
| | GSPGQSITISC | SCAASGFTFS | CAASGFTFSSY | FLFPPKPKDT |
| | TGTSSDVGGYN | SYIMMWVRQA | IMMWVRQAPGK | LMISRTPEVT |
| | YVSWYQQHPGK | PGKGLEWVSS | GLEWVSSIYPS | CVVVDVSHED |
| | APKLMIYDVSN | IYPSGGITFY | GGITFYADTVK | PEVKFNWYVD |
| | RPSGVSNRFSG | ADTVKGRFTI | GRFTISRDNSK | GVEVHNAKTK |
| | SKSGNTASLTI | SRDNSKNTLY | NTLYLQMNSLR | PREEQYNSTY |
| | SGLQAEDEADY | LQMNSLRAED | AEDTAVYYCAR | RVVSVLTVLH |
| | YCSSYTSSSTR | TAVYYCARIK | IKLGTVTTVDY | QDWLNGKEYK |
| | VFGTGTKVTVL | LGTVTTVDYW | WGQGTLVTVSS | CKVSNKALPA |
| | GQPKANPTVTL | GQGTLVTVSS | ASTKGPSVFPL | PIEKTISKAK |
| | FPPSSEELQAN | ASTKGPSVFP | APSSKSTSGGT | GQPREPQVCT |
| | KATLVCLISDF | LAPSSKSTSG | AALGCLVKDYF | LPPSRDELTK |
| | YPGAVTVAWKA | GTAALGCLVK | PEPVTVSWNSG | NQVSLSCAVD |
| | DGSPVKAGVET | DYFPEPVTVS | ALTSGVHTFPA | GFYPSDIAVE |
| | TKPSKQSNNKY | WNSGALTSGV | VLQSSGLYSLS | WESNGQPENN |
| | AASSYLSLTPE | HTFPAVLQSS | SVVTVPSSSLGT | YKTTPPVLDS |
| | QWKSHRSYSCQ | GLYSLSSVVTV | QTYICNVNHKP | DGSFFLVSKL |
| | VTHEGSTVEKT | PSSSLGTQTY | SNTKVDKKVEP | TVDKSRWQQG |
| | VAPTECS | ICNVNHKPSN | KSCDKTHTCPP | NVFSCSVMHE |
| | | TKVDKKVEPK | CPAPELLGGPS | ALHNHYTQKS |
| | | SCDKTHTCPP | VFLFPPKPKDT | LSLSPG |
| | | CPAPELLGGP | LMISRTP | |
| | | SVFLFPPKPK | EVTCWVDV | |
| | | DTLMISRTPE | SHEDPEVK | |
| | | VTCVVVDVSH | FNWYVDGV | |
| | | EDPE | EVHNAKTK | |
| | | VKFNWYVDGV | PREEQYNS | |
| | | EVHNAKTKPR | TYRVVSVL | |
| | | EEQYNSTYRV | TVLHQDWL | |
| | | VSVLTVLHQD | NGKEYKCK | |
| | | WLNGKEYKCK | VSNKALPA | |
| | | VSNKALPAPI | PIEKTISK | |
| | | EKTISKAKGQ | AKGQPREP | |
| | | PREPQVYTLP | QVYTLPPS | |
| | | PCRDKLTKNQ | RDELTKNQ | |
| | | VSLWCLVKGF | VSLTCLVK | |
| | | YPSDIAVEWE | GFYPSDIA | |
| | | SNGQPENNYK | VEWESNGQ | |
| | | TTPPVLDSDG | PENNYDTT | |
| | | SFFLYSKLTV | PPVLDSDG | |
| | | DKSRWQQGNV | SFFLYSDL | |
| | | FSCSVMHEAL | TVDKSRWQ | |
| | | HNHYTQKSLS | QGNVFSCS | |
| | | LSPGKGGGGG | VMHEALHN | |
| | | GGGGGGGGGG | HYTQKSLS | |
| | | GGGGGDKTHT | LSPG | |
| | | CPPCPAPELL | | |
| | | GGPSVFLFPP | | |
| | | KPKDTLMISR | | |
| | | TPEVTCVVVD | | |
| | | VSHEDPEVKF | | |
| | | NWYVDGVEVH | | |
| | | NAKTKPREEQ | | |
| | | YNSTYRVVSV | | |
| | | LTVLHQDWLN | | |
| | | GKEYKCKVSN | | |
| | | KALPAPIEKT | | |
| | | ISKAKGQPRE | | |
| | | PQVYTLPPCR | | |
| | | DKLTKNQVSL | | |
| | | WCLVKGFYPS | | |
| | | DIAVEWESNG | | |
| | | QPENNYKTTP | | |
| | | PVLDSDGSFF | | |
| | | LYSKLTVDKS | | |
| | | RWQQGNVFSC | | |
| | | SVMHEALHNH | | |
| | | YTQKSLSLSP | | |
| | | GKGGGGGGGG | | |
| | | GGGGGGGGGG | | |
| | | GGDKTHTCPP | | |
| | | CPAPELLGGP | | |
| | | SVFLFPPKPK | | |
| | | DTLMISRTPE | | |
| | | VTCVVVDVSH | | |
| | | EDPEVKFNWY | | |
| | | VDGVEVHNAK | | |
| | | TKPREEQYNS | | |
| | | TYRVVSVLTV | | |
| | | LHQDWLNGKE | | |
| | | YKCKVSNKAL | | |
| | | PAPIEKTISK | | |
| | | AKGQPREPQV | | |
| | | YTLPPSRKEL | | |
| | | TKNQVSLTCL | | |
| | | VKGFYPSDIA | | |
| | | VEWESNGQPE | | |
| | | NNYKTTPPVL | | |
| | | KSDGSFFLYS | | |
| | | KLTVDKSRWQ | | |
| | | QGNVFSCSVM | | |
| | | HEALHNHYTQ | | |
| | | KSLSLSPGQ | | |

Cell Culture

DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by multiple plasmids.

Protein Purification

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (PBS, pH 7.0) after loading and further washed with intermediate wash buffer 50 mM citrate buffer (pH 5.5) to remove additional process related impurities. The bound Fc construct material was eluted with 100 mM glycine, pH 3 and the eluate was quickly neutralized by the addition of 1 M TRIS pH 7.4 then centrifuged and sterile filtered through a 0.2 μm filter.

The proteins were further fractionated by ion exchange chromatography using Poros XS resin (Applied Biosciences). The column was pre-equilibrated with 50 mM MES, pH 6 (buffer A), and the sample was diluted (1:3) in the equilibration buffer for loading. The sample was eluted using a 12-15CV's linear gradient from 50 mM MES (100% A) to 400 mM sodium chloride, pH 6 (100% B) as the elution buffer. All fractions collected during elution were analyzed by analytical size exclusion chromatography (SEC) and target fractions were pooled to produce the purified Fc construct material.

After ion exchange, the target fraction was buffer exchanged into 1×-PBS buffer using a 30 kDa cut-off polyether sulfone (PES) membrane cartridge on a tangential flow filtration system. The samples were concentrated to approximately 10-15 mg/mL and sterile filtered through a 0.2 μm filter.

Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 5. Design and Purification of Fc-Antigen Binding Domain Construct 46 with an Anti-CD20 Antigen Binding Domain or an Anti-PD-L1 Antigen Binding Domain

An unbranched construct formed from tandem Fc domains (FIG. 13) was made as described below. Fc-antigen binding domain construct 46 (CD20) and construct 46 (PD-L1) each include three distinct Fc monomer containing polypeptides (a long Fc chain (SEQ ID NO: 241); a copy of a first short Fc chain (SEQ ID NO: 236); and two copies of a second short Fc chain that is an anti-CD20 short Fc chain (SEQ ID NO: 67) or an anti-PD-L1 Fc short chain (SEQ ID NO: 68)), and two copies of either an anti-CD20 light chain polypeptide (SEQ ID NO: 61) or an anti-PD-L1 light chain polypeptide (SEQ ID NO: 49), respectively. The long Fc chain contains three Fc domain monomers, each with a set of protuberance-forming mutations selected from Table 3 and/or one or more reverse charge mutation selected from Table 4, (the first Fc domain monomer with a different set of heterodimerization mutations than the second and third Fc domain monomers), in a tandem series. The first short Fc chain contains an Fc domain monomer with a first set of cavity-forming mutations selected from Table 3 and/or one or more reverse charge mutation selected from Table 4 (wherein the mutations are different from a second set of mutations in the second short Fc chain). The second short Fc chain contains an Fc domain monomer with a second set of cavity-forming mutations selected from Table 3 and/or one or more reverse charge mutation selected from Table 4 (wherein the mutations are different from the first set off mutations in the first short Fc chain), and an antigen binding domain at the N-terminus.

In this case, the long Fc chain contains one Fc domain monomer with D356K and D399K charge mutations in a tandem series with two Fc domain monomers with S354C and T366W protuberance-forming mutations and an E357K charge mutation. The first short Fc chain contains an Fc domain monomer with K392D and K409D charge mutations. The second short Fc chain contains an Fc domain monomer with Y349C, T366S, L368A, and Y407V cavity-forming mutations and a K370D charge mutation, and either anti-CD20 VH and CH1 domains (EU positions 1-220) at the N-terminus (construct 46 (CD20)) or anti-PD-L1 VH and CH1 domains (EU positions 1-220) at the N-terminus (construct 46 (PD-L1)).

TABLE 10
Construct 46 (CD20) and Construct 46 (PD-L1) sequences

| | | | | Second Short |
|---|---|---|---|---|
| | | | | Fc chain |
| | | | | (with |
| | | | | anti-CD20 |
| | | | | or anti- |
| | | | First Short | PD-L1 VH |
| Construct | Light chain | Long Fc chain | Fc chain | and CH1) |
| Construct | SEQ ID NO: 61 | SEQ ID NO: 241 | SEQ ID NO: 236 | SEQ ID NO: 67 |
| 46 (CD20) | DIVMTQTPLSLPVTP | DKTHTCPPCPAPEL | DKTHTCPPCPAP | QVQLVQSGAEVK |
| | GEPASISCRSSKSLL | LGGPSVFLFPPKPK | ELLGGPSVFLFP | KPGSSVKVSCKA |
| | HSNGITYLYWYLQKP | DTLMISRTPEVTCV | PKPKDTLMISRT | SGYAFSYSWINW |
| | GQSPQLLIYQMSNLV | VVDVSHEDPEVKFN | PEVTCVVVDVSH | VRQAPGQGLEWM |
| | SGVPDRFSGSGSGTD | WYVDGVEVHNAKTK | EDPEVKFNWYVD | GRIFPGDGDTDY |
| | FTLKISRVEAEDVGV | PREEQYNSTYRVVS | GVEVHNAKTKPR | NGKFKGRVTITA |
| | YYCAQNLELPYTFGG | VLTVLHQDWLNGKE | EEQYNSTYRVVS | DKSTSTAYMELS |
| | GTKVEIKRTVAAPSV | YKCKVSNKALPAPI | VLTVLHQDWLNG | SLRSEDTAVYYC |
| | FPIFPSDEQLKSGTA | EKTISKAKGQPREP | KEYKCKVSNKAL | ARNVFDGYWLVY |
| | SWCLLNNFYPREAKV | QVYTLPPCRDKLTK | PAPIEKTISKAK | WGQGTLVTVSSA |
| | QWKVDNALQSGNSQE | NQVSLWCLVKGFYP | GQPREPQVYTLP | STKGPSVFPLAP |
| | SVTEQDSKDSTYSLS | SDIAVEWESNGQPE | PSRDELTKNQVS | SSKSTSGGTAAL |
| | STLTLSKADYEKHKV | NNYKTTPPVLDSDG | LTCLVKGFYPSD | GCLVKDYFPEPV |
| | YACEVTHQGLSSPVT | SFFLYSKLTVDKSR | IAVEWESNGQPE | TVSWNSGALTSG |
| | KSFNRGEC | WQQGNVFSCSVMHE | NNYDTTPPVLDS | VHTFPAVLQSSG |
| | | ALHNHYTQKSLSLS | DGSFFLYSDLTV | LYSLSSVVTVPSS |
| | | PGKGGGGGGGGGGG | DKSRWQQGNVFS | SLGTQTYICNVN |
| | | GGGGGGGGGDKTHT | CSVMHEALHNHY | HKPSNTKVDKKV |
| | | CPPCPAPELLGGPS | TQKSLSLSPG | EPKSCDKTHTCP |
| | | VFLFPPKPKDTLMI | | PCPAPELLGGPS |
| | | SRTPEVTCVVVDVS | | VFLFPPKPKDTL |
| | | HEDPEVKFNWYVDG | | MISRTPEVTCVV |
| | | VEVHNAKTKPREEQ | | VDVSHEDPEVKF |
| | | YNSTYRVVSVLTVL | | NWYVDGVEVHNA |
| | | HQDWLNGKEYKCKV | | KTKPREEQYNST |
| | | SNKALPAPIEKTIS | | YRVVSVLTVLHQ |
| | | KAKGQPREPQVYTL | | DWLNGKEYKCKV |
| | | PPCRDKLTKNQVSL | | SNKALPAPIEKT |
| | | WCLVKGFYPSDIAV | | ISKAKGQPREPQ |
| | | EWESNGQPENNYKT | | VCTLPPSRDELT |
| | | TPPVLDSDGSFFLY | | KNQVSLSCAVDG |
| | | SKLTVDKSRWQQGN | | FYPSDIAVEWES |
| | | VFSCSVMHEALHNH | | NGQPENNYKTTP |
| | | YTQKSLSLSPGKGG | | PVLDSDGSFFLV |
| | | GGGGGGGGGGGGGG | | SKLTVDKSRWQQ |
| | | GGGGDKTHTCPPCP | | GNVFSCSVMHEA |
| | | APELLGGPSVFLFP | | LHNHYTQKSLSL |
| | | PKPKDTLMISRTPE | | SPG |
| | | VTCVVVDVSHEDPE | | |
| | | VKFNWYVDGVEVHN | | |
| | | AKTKPREEQYNSTY | | |
| | | RVVSVLTVLHQ | | |
| | | DWLNGKEYKCKVSN | | |
| | | KALPAPIEKTISKA | | |
| | | KGQPREPQVYTLPP | | |
| | | SRKELTKNQVSLTC | | |
| | | LVKGFYPSDIAVEW | | |
| | | ESNGQPENNYKTTP | | |
| | | PVLKSDGSFFLYSK | | |
| | | LTVDKSRWQQGNVF | | |
| | | SCSVMHEALHNHYT | | |
| | | QKSLSLSPGQ | | |
| Construct | SEQ ID NO: 49 | SEQ ID NO: 241 | SEQ ID NO: 236 | SEQ ID NO: 68 |
| 46 (PD-L1) | QSALTQPASVSGSPG | DKTHTCPPCPAPELLGGPS | DKTHTCPPCPAPELLGG | EVQLLESGGGLVQPGGS |
| | QSITISCTGTSSDVG | VFLFPPKPKDTLMISRTPE | PSVFLFPPKPKDTLMIS | LRLSCAASGFTFSSYIM |
| | GYNYVSWYQQHPGKA | VTCVVVDVSHEDPEVKFNW | RTPEVTCVVVDVSHEDP | MWVRQAPGKGLEWVSSI |
| | PKLMIYDVSNRPSGV | YVDGVEVHNAKTKPREEQY | EVKFNWYVDGVEVHNAK | YPSGGITFYADTVKGRF |
| | SNRFSGSKSGNTASL | NSTYRVVSVLTVLHQDWLN | TKPREEQYNSTYRVVSV | TISRDNSKNTLYLQMNS |
| | TISGLQAEDEADYYC | GKEYKCKVSNKALPAPIEK | LTVLHQDWLNGKEYKCK | LRAEDTAVYYCARIKLG |
| | SSYTSSSTRVFGTGT | TISKAKGQPREPQVYTLPP | VSNKALPAPIEKTISKA | TVTTVDYWGQGTLVTVS |
| | KVTVLGQPKANPTVT | CRDKLTKNQVSLWCLVKGF | KGQPREPQVYTLPPSRD | SASTKGPSVFPLAPSSK |
| | LFPPSSEELQANKAT | YPSDIAVEWESNGQPENNY | ELTKNQVSLTCLVKGFY | STSGGTAALGCLVKDYF |
| | LVCLISDFYPGAVTV | KTTPPVLDSDGSFFLYSKL | PSDIAVEWESNGQPENN | PEPVTVSWNSGALTSGV |
| | AWKADGSPVKAGVET | TVDKSRWQQGNVFSCSVMH | YDTTPPVLDSDGSFFLY | HTFPAVLQSSGLYSLSS |
| | TKPSKQSNNKYAASS | AELHNHYTQKSLSLSPGKG | SDLTVDKSRWQQGNVFS | VVTVPSSSLGTQTYICN |
| | YLSLTPEQWKSHRSY | GGGGGGGGGGGGGGGGG | CSVMHEALHNHYTQKSL | VNHKPSNTKVDKKVEPK |
| | SCQVTHEGSTVEKTV | GGDKTHTCPPCPAPELLGG | SLSPG | SCDKTHTCPPCPAPELL |
| | APTECS | PSVFLFPPKPKDTLMISRT | | GGPSVFLFPPKPKDTLM |
| | | PEVTCVVVDVSHEDPEVKF | | ISRTPEVTCVVVDVSHE |
| | | NWYVDGVEVHNAKTKPREE | | DPEVKFNWYVDGVEVHN |
| | | QYNSTYRVVSVLTVLHQD | | AKTKPREEQYNSTYRVV |
| | | WLNGKEYKCKVSNKALPAP | | SVLTVLHQDWLNGKEYK |
| | | IEKTISKAKGQPREPQVYT | | CKVSNKALPAPIEKTIS |
| | | LPPCRDKLTKNQVSLWCLV | | KAKGQPREPQVCTLPPS |
| | | KGFYPSDIAVEWESNGQPE | | RDELTKNQVSLSCAVDG |
| | | NNYKTTPPVLDSDGSFFLY | | FYPSDIAVEWESNGQPE |
| | | SKLTVDKSRWQQGNVFSCS | | NNYKTTPPVLDSDGSFF |
| | | VMHEALHNHYTQKSLSLSP | | LVSKLTVDKSRWQQGNV |
| | | GKGGGGGGGGGGGGGGGG | | CFSSVMHEALHNHYTQK |
| | | GGGGDKTHTCPPCPAPELL | | SLSLSPG |
| | | GGPSVFLFPPKPKDTLMIS | | |
| | | RTPEVTCVVVDVSHEDPEV | | |
| | | KFNWYVDGVEVHNAKTKPR | | |
| | | EEQYNSTYRVVSVLTVLHQ | | |
| | | DWLNGKEYKCKVSNKALPA | | |
| | | PIEKTISKAKGQPREPQVY | | |
| | | TLPPSRKELTKNQVSLTCL | | |
| | | VKGFYPSDIAVEWESNGQP | | |
| | | ENNYKTTPPVLKSDGSFFL | | |
| | | YSKLTVDKSRWQQGNVFSC | | |
| | | SVMHEALHNHYTQKSLSLS | | |
| | | PGQ | | |

Cell Culture

DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by multiple plasmids.

Protein Purification

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (PBS, pH 7.0) after loading and further washed with intermediate wash buffer 50 mM citrate buffer (pH 5.5) to remove additional process related impurities. The bound Fc construct material was eluted with 100 mM glycine, pH 3 and the eluate was quickly neutralized by the addition of 1 M TRIS pH 7.4 then centrifuged and sterile filtered through a 0.2 μm filter.

The proteins were further fractionated by ion exchange chromatography using Poros XS resin (Applied Biosciences). The column was pre-equilibrated with 50 mM MES, pH 6 (buffer A), and the sample was diluted (1:3) in the equilibration buffer for loading. The sample was eluted using a 12-15CV's linear gradient from 50 mM MES (100% A) to 400 mM sodium chloride, pH 6 (100% B) as the elution buffer. All fractions collected during elution were analyzed by analytical size exclusion chromatography (SEC) and target fractions were pooled to produce the purified Fc construct material.

After ion exchange, the target fraction was buffer exchanged into 1×-PBS buffer using a 30 kDa cut-off polyether sulfone (PES) membrane cartridge on a tangential flow filtration system. The samples were concentrated to approximately 10-15 mg/mL and sterile filtered through a 0.2 μm filter.

Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 6. Design and Purification of Fc-Antigen Binding Domain Construct 47 with an Anti-CD20 Antigen Binding Domain or an Anti-PD-L1 Antigen Binding Domain

Fc-antigen binding domain constructs are designed to increase folding efficiencies, to minimize uncontrolled association of subunits, which may create unwanted high molecular weight oligomers and multimers, and to generate compositions for pharmaceutical use that are substantially homogenous (e.g., at least 85%, 90%, 95%, 98%, or 99% homogeneous). With these goals in mind, an unbranched construct formed from tandem Fc domains (FIG. 14) was made as described below. Fc-antigen binding domain construct 47 (CD20) and construct 47 (PD-L1) each include three distinct Fc monomer containing polypeptides (a long Fc chain (SEQ ID NO: 243); two copies of a first short Fc chain that is an anti-CD20 short Fc chain (SEQ ID NO: 247) or an anti-PD-L1 Fc short chain (SEQ ID NO: 248); and a copy of a second short Fc chain (SEQ ID NO: 63)), and two copies of either an anti-CD20 light chain polypeptide (SEQ ID NO: 61) or an anti-PD-L1 light chain polypeptide (SEQ ID NO: 49), respectively. The long Fc chain contains three Fc domain monomers, each with a set of protuberance-forming mutations selected from Table 3 (heterodimerization mutations) and/or one or more reverse charge mutation selected from Table 4, (the third Fc domain monomer with a different set of heterodimerization mutations than the first and second Fc domain monomers) in a tandem series. The first short Fc chain contains an Fc domain monomer with a first set of cavity-forming mutations selected from Table 3 and/or one or more reverse charge mutation selected from Table 4 (wherein the mutations are different from a second set of mutations in the second short Fc chain), and an antigen binding domain at the N-terminus. The second short Fc chain contains an Fc domain monomer with a second set of cavity-forming mutations selected from Table 3 and/or one or more reverse charge mutation selected from Table 4 (wherein the mutations are different from the first set off mutations in the first short Fc chain).

In this case, the long Fc chain contains two Fc domain monomers, each with D356K and D399K charge mutations in a tandem series with an Fc domain monomer with S354C and T366W protuberance-forming mutations and a E357K charge mutation. The first short Fc chain contains an Fc domain monomer with a K392D and K409D charge mutations, and either anti-CD20 VH and CH1 domains (EU positions 1-220) at the N-terminus (construct 47 (CD20)) or anti-PD-L1 VH and CH1 domains (EU positions 1-220) at the N-terminus (construct 47 (PD-L1)). The second short Fc chain contains an Fc domain monomer with Y349C, T366S, L368A and Y407V cavity-forming mutations and a K370D charge mutation.

TABLE 11
Construct 47 (CD20) and Construct 47 (PD-L1) sequences

| | | | First Short | |
|---|---|---|---|---|
| | | | Fc chain | |
| | | | (with | |
| | | | anti-CD20 | |
| | | | or anti- | |
| | | | PD-L1 VH | Second Short |
| Construct | Light chain | Long Fc chain | and CH1) | Fc chain |
| Construct | SEQ ID NO: 61 | SEQ ID NO: 324 | SEQ ID NO: 247 | SEQ ID NO: 63 |
| 47 (CD20) | DIVMTQTPLSLPVTPG | DKTHTCPPCPAPELLGGP | QVQLVQSGAEVKKPGSSV | DKTHTCPPCPAPELLGGP |
| | EPASISCRSSKSLLHSN | SVFLFPPKPKDTLMISRTP | KVSCKASGYAFSYSWINW | SVFLFPPKPKDTLMISRT |
| | GITYLYWYLQKPGQSP | EVTCVVVDVSHEDPEVKF | VRQAPGQGLEWMGRIFP | PEVTCVVVDVSHEDPEVK |
| | QLUYQMSNLVSGVPDR | NWYVDGVEVHNAKTKP | GDGDTDYNGKFKGRVTIT | FNWYVDGVEVHNAKTKP |
| | FSGSGSGTDFTLKISR | REEQYNSTYRVVSVLTVL | ADKSTSTAYMELSSLRSED | REEQYNSTYRWSVLTVL |
| | VEAEDVGVYYCAQNLE | HQDWLNGKEYKCKVSNK | TAVYYCARNVFDGYWLVY | HQDWLNGKEYKCKVSNK |
| | LPYTFGGGTKVEIKRTV | ALPAPIEKTISKAKGQPR | WGQGTLVTVSSASTKGPS | ALPAPIEKTISKAKGQPR |
| | AAPSVFIFPPSDEQLKS | EPQVYTLPPCRDKLTKNQ | VFPLAPSSKSTSGGTAALG | EPQVCTLPPSRDELTKNQ |
| | GTASVVCLLNNFYPRE | VSLWCLVKGFYPSDIAVE | CLVKDYFPEPVTVSWNSG | VSLSCAVDGFYPSDIAVE |
| | AKVQWKVDNALQSGN | WESNGQPENNYKTTPPV | ALTSGVHTFPAVLQSSGLY | WESNGQPENNYKTTPPVL |
| | SQESVTEQDSKDSTYS | LDSDGSFFLYSKLTVDK | SLSSWTVPSSSLGTQTYIC | DSDGSFFLVSKLTVDKSR |
| | LSSTLTLSKADYEKHK | SRWQQGNVFSCSVMHEA | NVNHKPSNTKVDKKVEPK | WQQGNVFSCSVMHEAL |
| | VYACEVTHQGLSSPVT | LHNHYTQKSLSLSPGKG | SCDKTHTCPPCPAPELLGG | HNHYTQKSLSLSPG |
| | KSFNRGEC | GGGGGGGGGGGGGGGGG | PSVFLFPPKPKDTLMISRT | |
| | | GGDKTHTCPPCPAPELL | PEVTCVVVDVSHEDPEVKF | |
| | | GGPSVFLFPPKPKDTLMI | NWYVDGVEVHNAKTKPR | |
| | | SRTPEVTCVVVDVSHEDP | EEQYNSTYRVVSVLTVLHQ | |
| | | EVKFNWYVDGVEVHNA | DWLNGKEYKCKVSNKALP | |
| | | KTKPREEQYNSTYRVVSV | APIEKTISKAKGQPREPQV | |
| | | LTVLHQDWLNGKEYKCK | YTLPPSRDELTKNQVSLTCL | |
| | | VSNKALPAPIEKTISKAKG | VKGFYPSDIAVEWESNGQ | |
| | | QPREPQVYTLPPSRKELT | PENNYDTTPPVLDSDGSFF | |
| | | KNQVSLTCLVKGFYPSDI | LYSDLTVDKSRWQQGNVF | |
| | | AVEWESNGQPENNYKTT | SCSVMHEALHNHYTQKSL | |
| | | PPVLKSDGSFFLYSKLTV | SLSPG | |
| | | DKSRWQQGNVFSCSVMH | | |
| | | EALHNHYTQKSLSLSPGQ | | |
| | | KGGGGGGGGGGGGGG | | |
| | | GGGGGGDKTHTCPPCPA | | |
| | | PELLGGPSVFLFPPKPKDT | | |
| | | LMISRTPEVTCVVVDVSH | | |
| | | EDPEVKFNWYVDGVEV | | |
| | | HNAKTKPREEQYNSTYR | | |
| | | VVSVLTVLHQDWLNGKEY | | |
| | | KCKVSNKALPAPIEKTIS | | |
| | | KAKGQPREPQVYTLPPSR | | |
| | | KELTKNQVSLTCLVKGFY | | |
| | | PSDIAVEWESNGQPENNY | | |
| | | KTTPPVLKSDGSFFLYSK | | |
| | | LTVDKSRWQQGNVFSCS | | |
| | | VMHEALHNHYTQKSLSL | | |
| | | SPGQ | | |
| Construct | SEQ ID NO: 49 | SEQ ID NO: 243 | SEQ ID NO: 63 | SEQ ID NO: 63 |
| 47 (PD-L1) | QSALTQPASVSGSPGQ | EVQLLESGGGLVQPGGS | DKTHTCPPCPAPELLGGPS | DKTHTCPPCPAPELLGGP |
| | SITISCTGTSSDVGGYN | LRLSCAASGFTFSSYIMM | VFLFPPKPKDTLMISRTPE | SVFLFPPKPKDTLMISRTP |
| | YVSWYQQHPGKAPKL | WVRQAPGKGLEWVSSIY | VTCVVVDVSHEDPEVKFN | EVTCVVVDVSHEDPEVKF |
| | MIYDVSNRPSGVSNRF | PSGGITFYADTVKGRFTI | WYVDGVEVHNAKTKPRE | NWYVDGVEVHNAKTKP |
| | SGSKSGNTASLTISGLQ | SRDNSKNTLYLQMNSLRA | EQYNSTYRVVSVLTVLHQ | REEQYNSTYRVVSVLTVL |
| | AEDEADYYCSSYTSSST | EDTAVYYCARIKLGTVTT | DWLNGKEYKCKVSNKALP | HQDWLNGKEYKCKVSN |
| | RVFGTGTKVTVLGQPK | VDYWGQGTLVTVSSAST | APIEKTISKAKGQPREPQV | KALPAPIEKTISKAKGQPR |
| | ANPTVTLFPPSSEELQA | KGPSVFPLAPSSKSTSGG | CTLPPSRDELTKNQVSLSC | EPQVCTLPPSRDELTKNQ |
| | NKATLVCLISDFYPGAV | TAALGCLVKDYFPEPVTV | AVDGFYPSDIAVEWESNG | VSLSCAVDGFYPSDIAVE |
| | TVAWKADGSPVKAGV | SWNSGALTSGVHTFPAV | QPENNYKTTPPVLDSDGS | WESNGQPENNYKTTPPV |
| | ETTKPSKQSNNKYAAS | LQSSGLYSLSSVVTVPSSS | FFLVSKLTVDKSRWQQGN | LDSDGSFFLVSKLTVDKSR |
| | SYLSLTPEQWKSHRSY | LGTQTYICNVNHKPSNTK | VFSCSVMHEALHNHYTQK | WQQGNVFSCSVMHEAL |
| | SCQVTHEGSTVEKTVA | VDKKVEPKSCDKTHTCPP | SLSLSPG | HNHYTQKSLSLSPG |
| | PTECS | CPAPELLGGPSVFLFPPKP | EVTCVVVDVSHEDPEVKF | |
| | | KDTLMISRTPEVTCVVVD | NWYVDGVEVHNAKTKPRE | |
| | | VSHEDPEVKFNWYVDGV | EQYNSTYRVVSVLTVLHQ | |
| | | EVHNAKTKPREEQYNSTY | DWLNGKEYKCKVSNKALP | |
| | | RVVSVLTVLHQDWLNGKE | APIEKTISKAKGQPREPQ | |
| | | YKCKVSNKALPAPIEKTI | VYTLPPSRDELTKNQVSL | |
| | | SKAKGQPREPQVYTLPPS | TCLVKGFYPSDIAVEWES | |
| | | RDELTKNQVSLTCLVKGF | NGQPENNYDTTPPVLDSD | |
| | | YPSDIAVEWESNGQPENN | GSFFLYSDLTVDKSRWQQ | |
| | | YKTTPPVLKSDGSFFLYS | GNVFSCSVMHEALHNHYT | |
| | | VKFNWYVDGVEVHNAKTKP | QKSLSLSPG | |
| | | REEQYNSTYRVVSVLTVLH | | |
| | | QDWLNGKEYKCKVSNKALP | | |
| | | APIEKTISKAKGQPREPQV | | |
| | | YTLPPCRDKLTKNQVSLWC | | |
| | | LVKGFYPSDIAVEWESNGQ | | |
| | | PENNYKTTPPVLDSDGSFF | | |
| | | LYSKLTVDKSRWQQGNVFS | | |
| | | CSVMHEALHNHYTQKSLSL | | |
| | | SPGKGGGGGGGGGGGGGG | | |
| | | GGGGGGDKTHTCPPCPAPE | | |
| | | LLGGPSVFLFPPKPKDTLM | | |
| | | ISRTPEVTCVVVDVSHEDP | | |
| | | EVKFNWYVDGVEVHNAKTK | | |
| | | PREEQYNSTYRVVSVLTVL | | |
| | | HQDWLNGKEYKCKVSNKAL | | |
| | | PAPIEKTISKAKGQPREPQ | | |
| | | VYTLPPCRDKLTKNQVSLW | | |
| | | CLVKGFYPSDIAVEWESNG | | |
| | | QPENNYKTTPPVLDSDGSF | | |
| | | FLYSKLTVDKSRWQQGNVF | | |
| | | SCSVMHEALHNHYTQKSLS | | |
| | | LSPGKGGGGGGGGGGGGGG | | |
| | | GGGGGGDKTHTCPPCPAPE | | |
| | | LLGGPSVFLFPPKPKDTLM | | |
| | | ISRTPEVTCVVVDVSHEDP | | |
| | | EVKFNWYVDGVEVHNAKTK | | |
| | | PREEQYNSTYRVVSVLTVL | | |
| | | HQDWLNGKEYKCKVSNKAL | | |
| | | PAPIEKTISKAKGQPREPQV | | |
| | | YTLPPSRKELTKNQVSLTCL | | |
| | | VKGFYPSDIAVEWESNGQPE | | |
| | | NNYKTTPPVLKSDGSFFLYS | | |
| | | KLTVDKSRWQQGNVFSCSVM | | |
| | | HEALHNHYTQKSLSLSPGQ | | |

Cell Culture

DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by multiple plasmids.

Protein Purification

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (PBS, pH 7.0) after loading and further washed with intermediate wash buffer 50 mM citrate buffer (pH 5.5) to remove additional process related impurities. The bound Fc construct material was eluted with 100 mM glycine, pH 3 and the eluate was quickly neutralized by the addition of 1 M TRIS pH 7.4 then centrifuged and sterile filtered through a 0.2 μm filter.

The proteins were further fractionated by ion exchange chromatography using Poros XS resin (Applied Biosciences). The column was pre-equilibrated with 50 mM MES, pH 6 (buffer A), and the sample was diluted (1:3) in the equilibration buffer for loading. The sample was eluted using a 12-15CV's linear gradient from 50 mM MES (100% A) to 400 mM sodium chloride, pH 6 (100% B) as the elution buffer. All fractions collected during elution were analyzed by analytical size exclusion chromatography (SEC) and target fractions were pooled to produce the purified Fc construct material.

After ion exchange, the target fraction was buffer exchanged into 1×-PBS buffer using a 30 kDa cut-off polyether sulfone (PES) membrane cartridge on a tangential flow filtration system. The samples were concentrated to approximately 10-15 mg/mL and sterile filtered through a 0.2 μm filter.

Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 7. Design and Purification of Fc-Antigen Binding Domain Construct 48 with an Anti-CD20 Antigen Binding Domain or an Anti-PD-L1 Antigen Binding Domain

An unbranched construct formed from tandem Fc domains (FIG. 15) is made as described below. Fc-antigen binding domain construct 48 (CD20) and construct 48 (PD-L1) each include three distinct Fc monomer containing polypeptides (a long Fc chain (SEQ ID NO: A); four copies of a first short Fc chain that is an anti-CD20 short Fc chain (SEQ ID NO: Y) or an anti-PD-L1 Fc short chain (SEQ ID NO: Y); and one copy of a second short Fc chain), and four copies of either an anti-CD20 light chain polypeptide (SEQ ID NO: 61) or an anti-PD-L1 light chain polypeptide (SEQ ID NO: 49), respectively. The long Fc chain contains five Fc domain monomers, each with a set of protuberance-forming mutations selected from Table 3 (heterodimerization mutations), and, optionally, one or more reverse charge mutation selected from Table 4, (the first, second, third, and fourth Fc domain monomers with a different set of heterodimerization mutations than the fifth Fc domain monomer) in a tandem series. The first short Fc chain contains an Fc domain monomer with a first set of cavity-forming mutations selected from Table 3 and, optionally, one or more reverse charge mutation selected from Table 4 (wherein the mutations are different from a second set of mutations in the second short Fc chain), and an antigen binding domain at the N-terminus. The second short Fc chain contains an Fc domain monomer with a second set of cavity-forming mutations selected from Table 3, and, optionally, one or more reverse charge mutation selected from Table 4 (wherein the mutations are different from the first set of mutations in the first short Fc chain).

In this case, the long Fc chain contains four Fc domain monomers with an E357K charge mutation and S354C and T366W protuberance-forming mutations (to promote heterodimerization), in a tandem series with one Fc domain monomer with K409D/D399K charge mutations (to promote heterodimerization). The first short Fc chain contains an Fc domain monomer with a K370D charge mutation and Y349C, T366S, L368A, and Y407V cavity-forming mutations (to promote heterodimerization), and either anti-CD20 VH and CH1 domains (EU positions 1-220) at the N-terminus (construct 48 (CD20)) or anti-PD-L1 VH and CH1 domains (EU positions 1-220) at the N-terminus (construct 48 (PD-L1)). The second short Fc chain contains an Fc domain monomer with K409D/D399K charge mutations (to promote heterodimerization).

Cell Culture

DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by multiple plasmids.

Protein Purification

The expressed proteins are purified from the cell culture supernatant by Protein A-based affinity column chromatography, using a Poros MabCapture A (LifeTechnologies) column. Captured Fc-antigen binding domain constructs are washed with phosphate buffered saline (PBS, pH 7.0) after loading and further washed with intermediate wash buffer 50 mM citrate buffer (pH 5.5) to remove additional process related impurities. The bound Fc construct material is eluted with 100 mM glycine, pH 3 and the eluate is quickly neutralized by the addition of 1 M TRIS pH 7.4 then centrifuged and sterile filtered through a 0.2 μm filter.

The proteins are further fractionated by ion exchange chromatography using Poros XS resin (Applied Biosciences). The column is pre-equilibrated with 50 mM MES, pH 6 (buffer A), and the sample is diluted (1:3) in the equilibration buffer for loading. The sample is eluted using a 12-15CV's linear gradient from 50 mM MES (100% A) to 400 mM sodium chloride, pH 6 (100% B) as the elution buffer. All fractions collected during elution is analyzed by analytical size exclusion chromatography (SEC) and target fractions were pooled to produce the purified Fc construct material.

After ion exchange, the target fraction is buffer exchanged into 1×-PBS buffer using a 30 kDa cut-off polyether sulfone (PES) membrane cartridge on a tangential flow filtration system. The samples are concentrated to approximately 10-15 mg/mL and sterile filtered through a 0.2 μm filter.

Non-Reducing Sodium Dodecyl Sulfate-Polyacrylamide Gel Electrophoresis (SDS-PAGE)

Samples are denatured in Laemmli sample buffer (4% SDS, Bio-Rad) at 95° C. for 10 min. Samples are run on a Criterion TGX stain-free gel (4-15% polyacrylamide, Bio-Rad). Protein bands are visualized by UV illumination or Coommassie blue staining. Gels are imaged by ChemiDoc MP Imaging System (Bio-Rad). Quantification of bands is performed using Imagelab 4.0.1 software (Bio-Rad).

### Example 9. Experimental Assays Used to Characterize Fc-Antigen Binding Domain Constructs Peptide and Glycopeptide Liquid Chromatography-MS/MS

The proteins (Fc constructs) were diluted to 1 μg/μL in 6M guanidine (Sigma). Dithiothreitol (DTT) was added to a concentration of 10 mM, to reduce the disulfide bonds under denaturing conditions at 65° C. for 30 min. After cooling on ice, the samples were incubated with 30 mM iodoacetamide (IAM) for 1 h in the dark to alkylate (carbamidomethylate) the free thiols. The protein was then dialyzed across a 10-kDa membrane into 25 mM ammonium bicarbonate buffer (pH 7.8) to remove IAM, DTT and guanidine. The protein was digested with trypsin in a Barocycler (NEP 2320; Pressure Biosciences, Inc.). The pressure was cycled between 20,000 psi and ambient pressure at 37° C. for a total of 30 cycles in 1 h. LC-MS/MS analysis of the peptides was performed on an Ultimate 3000 (Dionex) Chromatography System and an Q-Exactive (Thermo Fisher Scientific) Mass Spectrometer. Peptides were separated on a BEH PepMap (Waters) Column using 0.1% FA in water and 0.1% FA in acetonitrile as the mobile phases.

Intact Mass Spectrometry

50 μg of the protein (Fc construct) was buffer exchanged into 50 mM ammonium bicarbonate (pH 7.8) using 10 kDa spin filters (EMD Millipore) to a concentration of 1 μg/μL. 30 units PNGase F (Promega) was added to the sample and incubated at 37° C. for 5 hours. Separation was performed on a Waters Acquity C4 BEH column (1×100 mm, 1.7 um particle size, 300A pore size) using 0.1% FA in water and 0.1% FA in acetonitrile as the mobile phases. LC-MS was performed on an Ultimate 3000 (Dionex) Chromatography System and an Q-Exactive (Thermo Fisher Scientific) Mass Spectrometer. The spectra were deconvoluted using the default ReSpect method of Biopharma Finder (Thermo Fisher Scientific).

### Capillary Electrophoresis-Sodium Dodecyl Sulfate (CE-SDS) Assay

Samples were diluted to 1 mg/mL and mixed with the HT Protein Express denaturing buffer (PerkinElmer). The mixture was incubated at 40° C. for 20 min. Samples were diluted with 70 μL of water and transferred to a 96-well plate. Samples were analyzed by a Caliper GXII instrument (PerkinElmer) equipped with the HT Protein Express LabChip (PerkinElmer). Fluorescence intensity was used to calculate the relative abundance of each size variant.

### Non-Reducing SDS-PAGE

Samples are denatured in Laemmli sample buffer (4% SDS, Bio-Rad) at 95° C. for 10 min. Samples are run on a Criterion TGX stain-free gel (4-15% polyacrylamide, Bio-Rad). Protein bands are visualized by UV illumination or Coommassie blue staining. Gels are imaged by ChemiDoc MP Imaging System (Bio-Rad). Quantification of bands is performed using Imagelab 4.0.1 software (Bio-Rad).

### Complement Dependent Cytotoxicity (CDC)

CDC was evaluated by a colorimetric assay in which Raji cells (ATCC) were coated with serially diluted Rituximab, an Fc construct, or IVIg. Human serum complement (Quidel) was added to all wells at 25% v/v and incubated for 2 h at 37° C. Cells were incubated for 12 h at 37° C. after addition of WST-1 cell proliferation reagent (Roche Applied Science). Plates were placed on a shaker for 2 min and absorbance at 450 nm was measured.j

### Example 10. Complement-Dependent Cytotoxicity (CDC) Activation by Anti-CD20 Fc Constructs

A CDC assay was developed to test the degree to which anti-CD20 Fc constructs enhance CDC activity relative to an anti-CD20 monoclonal antibody, obinutuzumab. Anti-CD20 Fc constructs 45, 46, and 47 having the Fab sequence (VL+CL, VH+CH1) of Gazyva were produced as described in Examples 4, 5, and 6. Each anti-CD20 Fc construct, and the obinutuzumab monoclonal antibody, was tested in a CDC assay performed as follows:

Daudi cells grown in RPMI-1640 supplemented with 10% heat-inactivated FBS were pelleted, washed 1× with ice-cold PBS and resuspended in RPMI-1640 containing 0.1% BSA at a concentration of 1.0×10⁶ viable cells per mL. Fifty microliters of this cell suspension was added to all wells (except plate edges) of 96-well plates. Plates were kept on ice until all additions had been made. Test articles were serially diluted four-fold from a starting concentration of 450 nM in RPMI-1640+BSA. A total of ten concentrations was tested for each test article. Fifty microliters each was added to plated Daudi cells. Normal or C1q-depleted human complement serum (Quidel, San Diego, Calif.) was diluted 1:5 in RPMI-1640+BSA. Fifty microliters each was added to plated Daudi cells. Six normal serum control wells received cells, media only (no treatment) and 1/5 normal serum (Normal Background). Three of these wells also received 16.5 μL Triton X-100 (Promega, Madison, Wis.) (Normal Lysis Control). C1q-depleted Background and Lysis Controls were similarly prepared. PBS was added to all plate edge wells. Plates were incubated for 2 h at 37° C. After 2 h, 50 μL pre-warmed Alamar blue (Thermo, Waltham, Mass.) was added to all wells (expect plate edges). Plates were returned to the incubator overnight (18 h at 37° C.). After 18 h fluorescence was measured in a FlexStation 3. Plates were top-read using 544/590 Ex/Em filters and Auto Cut-Off. Means were calculated for Normal Background, Normal Lysis Control, C1q-depleted Background and C1q-depleted Lysis Control wells. Percent cell lysis was calculated as: Cell Lysis=(RFU Test−RFU Background)/(RFU Lysis Control−RFU Background)*100. The EC50 (nM) was determined for each construct.

As depicted in Table 12, anti-CD20 Fc constructs induced CDC in Daudi cells and demonstrated greater potency in enhancing cytotoxicity relative to the obinutuzumab monoclonal antibody, as evidenced by lower EC50 values.

TABLE 12
Potency of anti-CD20 Fc constructs to induce CDC in Daudi cells

| | | EC50 (nM) | | |
|---|---|---|---|---|
| Construct¹ | n | Range | Mean | SD |
| IgG1 Antibody, | 5 | 38-65 | 47 | 11 |
| Fucosylated | | | | |
| S3L-AA2-OBI | 2 | 0.50-0.57 | 0.54 | 0.046 |
| Construct 45 | | | | |
| (anti-CD20) | | | | |
| S3L-0AA2-OBI | 4 | 0.20-0.25 | 0.23 | 0.025 |
| Construct 46 | | | | |
| (anti-CD20) | | | | |
| S3L-0A22-OBI2 | 4 | 0.16-0.21 | 0.18 | 0.027 |
| Construct 47 | | | | |
| (anti-CD20) | | | | |

¹All constructs included G20 (SEQ ID NO: 23) linkers unless otherwise noted.

### Example 11. Complement-Dependent Cytotoxicity (CDC) Activation by Anti-PD-L1 Fc Constructs

A CDC assay was developed to test the degree to which anti-PD-L1 Fc constructs enhance CDC activity relative to an anti-PD-L1 monoclonal antibody, avelumab (Bavencio). Anti-PD-L1 Fc constructs 45, 46, and 47 having the Fab sequence (VL+CL, VH+CH1) of avelumab were produced as described in Examples 4, 5, and 6. Each anti-PD-L1 Fc construct, and the fucosylated and afucosylated avelumab monoclonal antibody, was tested in a CDC assay performed as follows:

The Human Embryonic Kidney (HEK) cell line transfected to stably express the human PD-L1 gene (CrownBio) were cultured in DMEM, 10% FBS, and 2 μg/mL puromycin as the selection marker. The cells were harvested and diluted in X-Vivo-15 media without genetecin or phenol red (Lonza). One hundred μl of HEK-PD-L1 cells at 6×10⁵ cells/mL were plated in a 96 well tissue culture treated flat bottom plate (BD Falcon). The Fc constructs and antibodies were serially diluted 1:3 in X-Vivo-15 media. Fifty μL of the diluted constructs were added to the wells on top of the target cells. Fifty μl of undiluted Human Serum Complement (Quidel Corporation) were added to each of the wells. The assay plate was then incubated for 2 h at 37° C. After the 2 h incubation 20 μL of WST-1 Cell Proliferation Reagent (Roche Diagnostics Corp) were added to each well and incubated overnight at 37° C. The next morning the assay plate was placed on a plate shaker for 2-5 min. Absorbance was measured at 450 nm with correction at 600 nm on a spectrophotometer (Molecular Devices SPECTRAmax M2). The EC50 (nM) was determined for each construct.

As depicted in Table 13, anti-PD-L1 Fc construct 47 induced CDC in HEK cells that express human PD L1, although the remaining anti-PD-L1 Fc constructs and the avelumab monoclonal antibody did not appear to induce CDC using this assay.

TABLE 13
Potency of anti-PD-L1 Fc constructs to induce CDC
in PD-L1 expressing HEK cells

| | | EC50 (nM) | | |
|---|---|---|---|---|
| Construct¹ | n | Range | Mean | SD |
| IgG1 Antibody, | 7 | No CDC | No CDC | N/A |
| Fucosylated | | activity² | activity² | |
| IgG1 Antibody, | 1 | No CDC | No CDC | N/A |
| Afucosylated | | activity2 | activity2 | |
| S3L-AA2-AVE | 1 | No CDC | No CDC | N/A |
| Construct 45 | | activity² | activity² | |
| (anti-PD-L1) | | | | |
| S3L-AA2-2AVE | 1 | Not | Not | N/A |
| Construct 46 | | determined | determined | |
| (anti-PD-L1) | | | | |
| S3L-A22-2AVE | 2 | 1.4-2.7 | 1.6 | 1.1 |
| Construct 47 | | | | |
| (anti-PD-L1) | | | | |

¹All constructs included G20 (SEQ ID NO: 23) linkers unless otherwise noted.
²Construct did not produce measurable CDC under the assay conditions.

### Example 12. Antibody-Dependent Cellular Phagocytosis (ADCP) Activation by Anti-CD20 Fc Constructs

ADCP Reporter Assay

An ADCP reporter assay was developed to test the degree to which anti-CD20 Fc constructs activate FcγRIIa signaling, thereby enhancing ADCP activity, relative to an anti-CD20 monoclonal obinutuzumab antibody (Gazyva). Anti-CD20 Fc constructs 45, 46, and 47 having the Fab sequence (VL+CL, VH+CH1) of Gazyva were produced as described in Examples 4, 5, and 6. Each anti-CD20 Fc construct, and fucosylated and afucosylated obinutuzumab monoclonal antibodies, were tested in an ADCC reporter assay performed as follows:

Raji target cells (1.5×10⁴ cells/well) and Jurkat/FcγRIla-H effector cells (Promega) (3.5×10⁴ cells/well) were resuspended in RPMI 1640 Medium supplemented with 4% low IgG serum (Promega) and seeded in a 96-well plate with serially diluted anti-CD20 Fc constructs. After incubation for 6 h at 37° C. in 5% CO₂, the luminescence was measured using the Bio-Glo Luciferase Assay Reagent (Promega) according to the manufacturer's protocol using a PHERAstar FS luminometer (BMG LABTECH).

As depicted in Table 14, anti-CD20 Fc constructs induced FcγRIIa signaling in an ADCP reporter assay and demonstrated greater potency in enhancing ADCP activity relative to the obinutuzumab monoclonal antibody, as evidenced by lower EC50 values.

TABLE 14
Potency of anti-CD20 Fc constructs to induce
FcγRIIa signaling in an ADCP reporter assay

| | | EC50 (nM) | | |
|---|---|---|---|---|
| Construct¹ | n | Range | Mean | SD |
| IgG1 Antibody, | 6 | 4.5-10.8 | 7.1 | 2.2 |
| Fucosylated | | | | |
| IgG1 Antibody, | 3 | 5.5-6.1 | 5.8 | 0.3 |
| Afucosylated | | | | |
| S3L-AA2-OBI | 1 | 0.13 | 0.13 | N/A |
| Construct 45 | | | | |
| (anti-CD20) | | | | |
| S3L-0AA2-OBI | 1 | 0.17 | 0.17 | N/A |
| Construct 46 | | | | |
| (anti-CD20) | | | | |
| S3L-0A22-OBI2 | 1 | 0.08 | 0.08 | N/A |
| Construct 47 | | | | |
| (anti-CD20) | | | | |

¹All constructs included G20 (SEQ ID NO: 23) linkers unless otherwise noted.

### Example 13. Antibody-Dependent Cellular Phagocytosis (ADCP) Activation by Anti-PD-L1 Fc Constructs

ADCP Reporter Assay

An ADCP reporter assay was developed to test the degree to which anti-PD-L1 Fc constructs activate FcγRIIa signaling, thereby enhancing ADCP activity, relative to an anti-PD-L1 monoclonal antibody, avelumab (Bavencio). Anti-PD-L1 Fc constructs 45, 46, and 47 having the Fab sequence (VL+CL, VH+CH1) of avelumab were produced as described in Examples 4, 5, and 6. Each anti-PD-L1 Fc construct, and fucosylated and afucosylated avelumab monoclonal antibodies, were tested in an ADCC reporter assay performed as follows:

Target HEK-PD-L1 cells (1.5×10⁴ cells/well) and effector Jurkat/FcγRIIa-H cells (Promega) (3.5×10⁴ cells/well) were resuspended in RPMI 1640 Medium supplemented with 4% low IgG serum (Promega) and seeded in a 96-well plate with serially diluted anti-PD-L1 Fc constructs. After incubation for 6 hours at 37° C. in 5% CO₂, the luminescence was measured using the Bio-Glo Luciferase Assay Reagent (Promega) according to the manufacturer's protocol using a PHERAstar FS luminometer (BMG LABTECH).

As depicted in Table 15, anti-PD-L1 Fc constructs induced FcγRIIa signaling in an ADCP reporter assay.

TABLE 15
Potency of anti-PD-L1 Fc constructs to induce
FcγRIIa signaling in an ADCP reporter assay

| Construct | | EC50 (nM) | | |
|---|---|---|---|---|
| Number¹ | n | Range | Mean | SD |
| IgG1 Antibody, | 6 | No | No | N/A |
| Fucosylated | | effect² | effect² | |
| IgG1 Antibody, | 1 | No | No | N/A |
| Afucosylated | | effect² | effect² | |
| S3L-AA2-AVE | 1 | 0.031 | 0.031 | N/A |
| Construct 45 | | | | |
| (anti-PD-L1) | | | | |
| S3L-AA2-2AVE | 1 | 0.03 | 0.03 | N/A |
| Construct 46 | | | | |
| (anti-PD-L1) | | | | |
| S3L-A22-2AVE | 1 | 0.03 | 0.03 | N/A |
| Construct 47 | | | | |
| (anti-PD-L1) | | | | |

¹All constructs included G20 (SEQ ID NO: 23) linkers unless otherwise noted.
²Construct did not induce measurable FcγRIIa signaling under the assay conditions.

### Example 14. Antibody-Dependent Cell-Mediated Cytotoxicity (ADCC) Activation by Anti-CD20 Fc Constructs

### ADCC Reporter Assay

An ADCC reporter assay was developed to test the degree to which anti-CD20 Fc constructs induce FcγRIIIa signaling and enhance ADCC activity relative to an anti-CD20 monoclonal antibody obinutuzumab (Gazyva). Anti-CD20 Fc constructs 45, 46, and 47 having the Fab sequence (VL+CL, VH+CH1) of Gazyva were produced as described in Examples 4, 5, and 6. Each anti-CD20 Fc construct and fucosylatedobinutuzumab monoclonal antibody were tested in an ADCC reporter assay performed as follows:

Raji target cells (1.25×10⁴ cells/well) and Jurkat/FcγRIIIa effector cells (Promega) (7.45×104 cells/well) were resuspended in RPMI 1640 Medium supplemented with 4% low IgG serum (Promega) and seeded in a 96-well plate with serially diluted anti-CD20 Fc constructs. After incubation for 6 hours at 37° C. in 5% CO2, the luminescence was measured using the Bio-Glo Luciferase Assay Reagent (Promega) according to the manufacturer's protocol using a PHERAstar FS luminometer (BMG LABTECH).

As depicted in Table 16, the anti-CD20 Fc constructs induced FcγRIIIa signaling in an ADCC reporter assay.

TABLE 16
Potency of anti-CD20 Fc constructs to induce
FcγRIIIa signaling in an ADCC reporter assay

| | | EC50 (nM) | | |
|---|---|---|---|---|
| Construct¹ | n | Range | Mean | SD |
| IgG1 Antibody, | 6 | 0.039-0.15 | 0.08 | 0.04 |
| Fucosylated | | | | |
| S3L-AA2-OBI | 1 | 0.055 | 0.055 | N/A |
| Construct 45 | | | | |
| (anti-CD20) | | | | |
| S3L-0AA2-OBI | 1 | 0.09 | 0.09 | N/A |
| Construct 46 | | | | |
| (anti-CD20) | | | | |
| S3L-0A22-OBI2 | 1 | 0.043 | 0.043 | N/A |
| Construct 47 | | | | |
| (anti-CD20) | | | | |

¹All constructs included G20 (SEQ ID NO: 23) linkers unless otherwise noted.

### Example 15. Antibody-Dependent Cell-Mediated Cytotoxicity (ADCC) Activation by Anti-PD-L1 Fc Constructs

ADCC Reporter Assay

An ADCC reporter assay was developed to test the degree to which anti-PD-L1 Fc constructs induce FcγRIIIa signaling and enhance ADCC activity relative to an anti-PD-L1 monoclonal antibody, avelumab (Bavencio). Anti-PD-L1 Fc constructs 45, 46, and 47 having the Fab sequence (VL+CL, VH+CH1) of avelumab were produced as described in Examples 4, 5, and 6. Each anti-PD-L1 Fc construct, and fucosylated and afucosylated avelumab monoclonal antibodies, were tested in an ADCC reporter assay performed as follows:

Target HEK-PD-L1 cells (1.25×10⁴ cells/well) and effector Jurkat/FcγRIIIa cells (Promega) (7.45×10⁴ cells/well) were resuspended in RPMI 1640 Medium supplemented with 4% low IgG serum (Promega) and seeded in a 96-well plate with serially diluted anti-PD-L1 constructs. After incubation for 6 hours at 37° C. in 5% CO₂, the luminescence was measured using the Bio-Glo Luciferase Assay Reagent (Promega) according to the manufacturer's protocol using a PHERAstar FS luminometer (BMG LABTECH).

As depicted in Table 17, some of the anti-PD-L1 Fc constructs induced FcγRIIIa signaling in an ADCC reporter assay. Induction of FcγRIIIa signaling could not be determined for Fc constructs 44, 45, and 47 and the afucosylated monoclonal antibody using this assay.

TABLE 17
Potency of anti-PD-L1 Fc constructs to induce
FcγRIIIa signaling in an ADCC reporter assay

| Construct | | EC50 (nM) | | |
|---|---|---|---|---|
| Number¹ | n | Range | Mean | SD |
| IgG1 Antibody, | 5 | 0.037-0.056 | 0.049 | 0.008 |
| Fucosylated | | | | |
| IgG1 Antibody, | 1 | Not | Not | N/A |
| Afucosylated | | determined² | determined² | |
| S3L-AA2-AVE | 1 | Not | Not | N/A |
| Construct 45 | | determined² | determined² | |
| (anti-PD-L1) | | | | |
| S3L-AA2-2AVE | 1 | 0.029 | 0.029 | N/A |
| Construct 46 | | | | |
| (anti-PD-L1) | | | | |
| S3L-A22-2AVE | 1 | Not | Not | N/A |
| Construct 47 | | determined² | determined² | |
| (anti-PD-L1) | | | | |

¹All constructs included G20 (SEQ ID NO: 23) linkers unless otherwise noted.
²Data could not be reliably fit to a four parameter logistic (4PL) curve.

### Example 16: Alternative Asymmetrically Branched Fc-Antigen Binding Domain Constructs

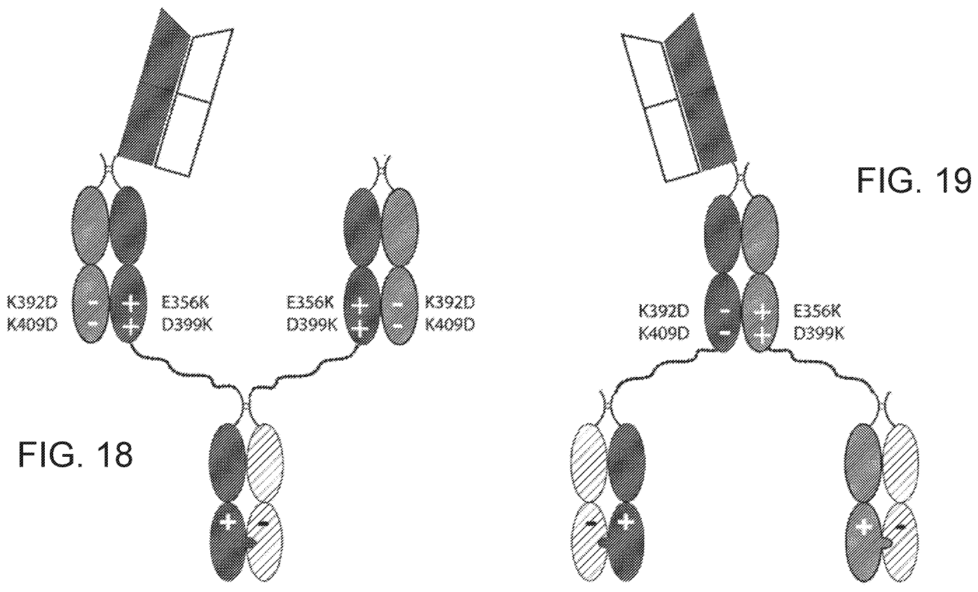

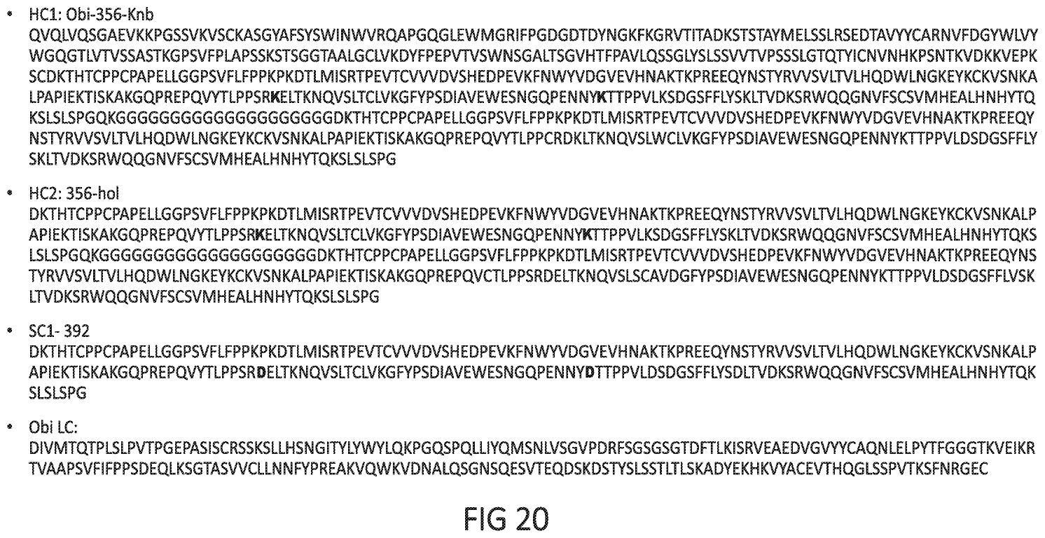

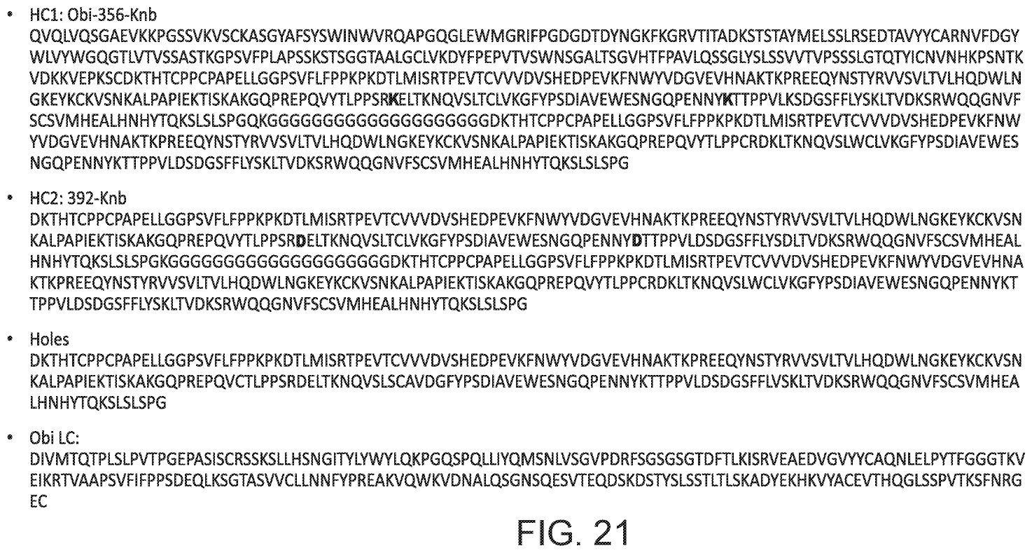

The two Fc constructs in FIG. 8 and FIG. 9 each have three Fc domains and were assembled from three different polypeptides using two sets of heterodimerization domain mutations. Both constructs are branched Fc constructs with a symmetrical distribution of Fc domains using an asymmetrical arrangement of polypeptide chains, and each has a single anti-CD20 Fab domain that is asymmetrically distributed on the construct. FIGS. 18 and 19 depict alternatives to the constructs of FIGS. 8 and 9, respectively in which the relative positions of the Fc domain(s) with the knobs-into-holes mutations in combination with an electrostatic steering mutations and the Fc domain(s) with the electrostatic steering mutations only are swapped. FIGS. 20 and 21 present the sequences of the polypeptides.

### OTHER EMBODIMENTS

All publications, patents, and patent applications mentioned in this specification are incorporated herein by reference to the same extent as if each independent publication or patent application was specifically and individually indicated to be incorporated by reference.

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims.

Other embodiments are within the claims.

What is claimed is:

1. A polypeptide comprising an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain,
wherein at least one Fc domain monomer comprises mutations forming an engineered protuberance,
and wherein at least one other Fc domain monomer comprises at least one, two or three reverse charge mutations.

2.-59. (canceled)

60. A polypeptide complex comprising a polypeptide of claim 1 joined to a second polypeptide comprising an IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

61.-64. (canceled)

65. The polypeptide complex of claim 60, wherein the polypeptide complex is further joined to a third polypeptide comprising an IgG1 Fc domain monomer comprising a hinge domain, a CH2 domain and a CH3 domain, wherein the polypeptide and the third polypeptide are joined by disulfide bonds between cysteine residues within the hinge domain of the first, second or third IgG1 Fc domain monomer of the polypeptide and the hinge domain of the third polypeptide, wherein the second and third polypeptides join to different IgG1 Fc domain monomers of the polypeptide.

66.-68. (canceled)

69. The polypeptide complex of claim 60 wherein the second polypeptide comprises the amino acid sequence of any of SEQ ID NOs: 42, 43, 45, and 47 having up to 10 single amino acid substitutions.

70. The polypeptide complex of claim 65 wherein the third polypeptide comprises the amino acid sequence of any of SEQ ID NOs: 42, 43, 45, and 47 having up to 10 single amino acid substitutions.

71.-77. (canceled)

78. A polypeptide comprising a first IgG1 Fc domain monomer comprising a hinge domain, a CH2 domain and a CH3 domain; a first linker; a second IgG1 Fc domain monomer comprising a hinge domain, a CH2 domain and a CH3 domain; an optional second linker; and an optional third IgG1 Fc domain monomer comprising a hinge domain, a CH2 domain and a CH3 domain,
wherein at least one Fc domain monomer comprises mutations forming an engineered protuberance,
and wherein at least one other Fc domain monomer comprises at least one, two or three reverse charge mutations.

79.-117. (canceled)

118. The polypeptide of claim 78 wherein each of the Fc domain monomers independently comprises the amino acid sequence of any of SEQ ID NOs:42, 43, 45, and 47 having up to 10 single amino acid substitutions.

119.-127. (canceled)

128. The polypeptide complex of claim 78, wherein the polypeptide complex is further joined to a third polypeptide comprising an IgG1 Fc domain monomer comprising a hinge domain, a CH2 domain and a CH3 domain, wherein the polypeptide and the third polypeptide are joined by disulfide bonds between cysteine residues within the hinge domain of the first, second or third IgG1 Fc domain monomer of the polypeptide and the hinge domain of the third polypeptide, wherein the second and third polypeptides join to different IgG1 Fc domain monomers of the polypeptide.

129.-157. (canceled)

158. The polypeptide complex of claim 78 comprising enhanced effector function in an antibody-dependent cytotoxicity (ADCC) assay, an antibody-dependent cellular phagocytosis (ADCP) and/or complement-dependent cytotoxicity (CDC) assay relative to a polypeptide complex having a single Fc domain and at least one antigen binding domain.

159. A nucleic acid molecule encoding the polypeptide of claim 1.

160. An expression vector comprising the nucleic acid molecule of claim 159.

161. A host cell comprising the nucleic acid molecule of claim 159.

162. A host cell comprising the expression vector of claim 160.

163. A method of producing the polypeptide of claim 1 comprising culturing the host cell of claim 161 under conditions to express the polypeptide.

164.-170. (canceled)

171. A pharmaceutical composition comprising the polypeptide of claim 1.

172. (canceled)

173. An Fc-antigen binding domain construct comprising:
a) a first polypeptide comprising
i) a first Fc domain monomer,
ii) a second Fc domain monomer,
iii) a third Fc domain monomer,
iii) a linker joining the first Fc domain monomer and the second Fc domain monomer; and
iv) a linker joining the second Fc domain monomer to the third Fc domain monomer;
b) a second polypeptide comprising a fourth Fc domain monomer;
c) a third polypeptide comprising a fifth Fc domain monomer; and
d) an antigen binding domain joined to the first polypeptide and to the third polypeptide;
wherein the first Fc domain monomer and the fourth Fc domain monomer combine to form a first Fc domain;
wherein the second Fc domain monomer and the fourth Fc domain monomer combine to form a second Fc domain; and
wherein the third Fc domain monomer and the fifth Fc domain monomer combine to form a third Fc domain.

174.-177. (canceled)

178. The Fc-antigen binding domain construct of claim 175, wherein each of the Fc domain monomers independently comprises the amino acid sequence of any of SEQ ID NOs:42, 43, 45, and 47 having up to 10 single amino acid substitutions.

179.-194. (canceled)

195. An Fc-antigen binding domain construct comprising:
a) a first polypeptide comprising
i) a first Fc domain monomer,
ii) a second Fc domain monomer,
iii) a third Fc domain monomer,
iii) a linker joining the first Fc domain monomer and the second Fc domain monomer; and
iv) a linker joining the second Fc domain monomer to the third Fc domain monomer;
b) a second polypeptide comprising a fourth Fc domain monomer;
c) a third polypeptide comprising a fifth Fc domain monomer; and
d) an antigen binding domain joined to the first polypeptide and to the second polypeptide;
wherein the first Fc domain monomer and the fourth Fc domain monomer combine to form a first Fc domain;
wherein the second Fc domain monomer and the fourth Fc domain monomer combine to form a second Fc domain; and
wherein the third Fc domain monomer and the fifth Fc domain monomer combine to form a third Fc domain.

196.-199. (canceled)

200. The Fc-antigen binding domain construct of claim 197, wherein each of the Fc domain monomers independently comprises the amino acid sequence of any of SEQ ID NOs:42, 43, 45, and 47 having up to 10 single amino acid substitutions.

201.-216. (canceled)

217. An Fc-antigen binding domain construct comprising:
a) a first polypeptide comprising
i) a first Fc domain monomer,
ii) a second Fc domain monomer,
iii) a third Fc domain monomer,
iii) a linker joining the first Fc domain monomer and the second Fc domain monomer; and
iv) a linker joining the second Fc domain monomer to the third Fc domain monomer;
b) a second polypeptide comprising a fourth Fc domain monomer;
c) a third polypeptide comprising a fifth Fc domain monomer; and
d) an antigen binding domain joined to the third polypeptide;
wherein the first Fc domain monomer and the fourth Fc domain monomer combine to form a first Fc domain;
wherein the second Fc domain monomer and the fifth Fc domain monomer combine to form a second Fc domain; and
wherein the third Fc domain monomer and the fifth Fc domain monomer combine to form a third Fc domain.

218.-221. (canceled)

222. The Fc-antigen binding domain construct of claim 219, wherein each of the Fc domain monomers independently comprises the amino acid sequence of any of SEQ ID NOs:42, 43, 45, and 47 having up to 10 single amino acid substitutions.

223.-244. (canceled)


SEQUENCE LISTING (327 sequences)

SEQ ID NO: 1 - protein, 5 aa, Artificial Sequence
  Description of Artificial Sequence: Synthetic peptide
GGGGS

SEQ ID NO: 2 - protein, 4 aa, Artificial Sequence
  Description of Artificial Sequence: Synthetic peptide
GGSG

SEQ ID NO: 3 - protein, 4 aa, Artificial Sequence
  Description of Artificial Sequence: Synthetic peptide
SGGG

SEQ ID NO: 4 - protein, 4 aa, Artificial Sequence
  Description of Artificial Sequence: Synthetic peptide
GSGS

SEQ ID NO: 5 - protein, 6 aa, Artificial Sequence
  Description of Artificial Sequence: Synthetic peptide
GSGSGS

SEQ ID NO: 6 - protein, 8 aa, Artificial Sequence
  Description of Artificial Sequence: Synthetic peptide
GSGSGSGS

SEQ ID NO: 7 - protein, 10 aa, Artificial Sequence
  Description of Artificial Sequence: Synthetic peptide
GSGSGSGSGS

SEQ ID NO: 8 - protein, 12 aa, Artificial Sequence
  Description of Artificial Sequence: Synthetic peptide
GSGSGSGSGSGS

SEQ ID NO: 9 - protein, 6 aa, Artificial Sequence
  Description of Artificial Sequence: Synthetic peptide
GGSGGS

SEQ ID NO: 10 - protein, 9 aa, Artificial Sequence
  Description of Artificial Sequence: Synthetic peptide
GGSGGSGGS

SEQ ID NO: 11 - protein, 12 aa, Artificial Sequence
  Description of Artificial Sequence: Synthetic peptide
GGSGGSGGSGGS

SEQ ID NO: 12 - protein, 8 aa, Artificial Sequence
  Description of Artificial Sequence: Synthetic peptide
GGSGGGSG

SEQ ID NO: 13 - protein, 12 aa, Artificial Sequence
  Description of Artificial Sequence: Synthetic peptide
GGSGGGSGGGSG

SEQ ID NO: 14 - protein, 16 aa, Artificial Sequence
  Description of Artificial Sequence: Synthetic peptide
GGSGGGSGGGSGGGSG

SEQ ID NO: 15 - protein, 20 aa, Artificial Sequence
  Description of Artificial Sequence: Synthetic peptide
GGSGGGSGGGSGGGSGGGSG

SEQ ID NO: 16 - protein, 10 aa, Artificial Sequence
  Description of Artificial Sequence: Synthetic peptide
GGGGSGGGGS

SEQ ID NO: 17 - protein, 15 aa, Artificial Sequence
  Description of Artificial Sequence: Synthetic peptide
GGGGSGGGGSGGGGS

SEQ ID NO: 18 - protein, 20 aa, Artificial Sequence
  Description of Artificial Sequence: Synthetic peptide
SGGGSGGGSGGGSGGGSGGG

SEQ ID NO: 19 - protein, 4 aa, Artificial Sequence
  Description of Artificial Sequence: Synthetic peptide
GGGG

SEQ ID NO: 20 - protein, 8 aa, Artificial Sequence
  Description of Artificial Sequence: Synthetic peptide
GGGGGGGG

SEQ ID NO: 21 - protein, 12 aa, Artificial Sequence
  Description of Artificial Sequence: Synthetic peptide
GGGGGGGGGGGG

SEQ ID NO: 22 - protein, 16 aa, Artificial Sequence
  Description of Artificial Sequence: Synthetic peptide
GGGGGGGGGGGGGGGG

SEQ ID NO: 23 - protein, 20 aa, Artificial Sequence
  Description of Artificial Sequence: Synthetic peptide
GGGGGGGGGGGGGGGGGGGG

SEQ ID NO: 24 - protein, 5 aa, Artificial Sequence
  Description of Artificial Sequence: Synthetic peptide
GGGGG

SEQ ID NO: 25 - protein, 10 aa, Artificial Sequence
  Description of Artificial Sequence: Synthetic peptide
GGGGGGGGGG

SEQ ID NO: 26 - protein, 15 aa, Artificial Sequence
  Description of Artificial Sequence: Synthetic peptide
GGGGGGGGGGGGGGG

SEQ ID NO: 27 - protein, 20 aa, Artificial Sequence
  Description of Artificial Sequence: Synthetic peptide
GGGGGGGGGGGGGGGGGGGG

SEQ ID NO: 28 - protein, 10 aa, Artificial Sequence
  Description of Artificial Sequence: Synthetic peptide
GENLYFQSGG

SEQ ID NO: 29 - protein, 8 aa, Artificial Sequence
  Description of Artificial Sequence: Synthetic peptide
SACYCELS

SEQ ID NO: 30 - protein, 5 aa, Artificial Sequence
  Description of Artificial Sequence: Synthetic peptide
RSIAT

SEQ ID NO: 31 - protein, 17 aa, Artificial Sequence
  Description of Artificial Sequence: Synthetic peptide
RPACKIPNDLKQKVMNH

SEQ ID NO: 32 - protein, 36 aa, Artificial Sequence
  Description of Artificial Sequence: Synthetic polypeptide
GGSAGGSGSGSSGGSSGASGTGTAGGTGSGSGTGSG

SEQ ID NO: 33 - protein, 17 aa, Artificial Sequence
  Description of Artificial Sequence: Synthetic peptide
AAANSSIDLISVPVDSR

SEQ ID NO: 34 - protein, 36 aa, Artificial Sequence
  Description of Artificial Sequence: Synthetic polypeptide
GGSGGGSEGGGSEGGGSEGGGSEGGGSEGGGSGGGS

SEQ ID NO: 35 - protein, 12 aa, Artificial Sequence
  Description of Artificial Sequence: Synthetic peptide
GGGSGGGSGGGS

SEQ ID NO: 36 - protein, 18 aa, Artificial Sequence
  Description of Artificial Sequence: Synthetic peptide
GGSGGGSGGGSGGGSGGS

SEQ ID NO: 37 - protein, 11 aa, Unknown
  Description of Unknown: Albumin binding peptide
DICLPRWGCLW

SEQ ID NO: 38 - protein, 6 aa, Artificial Sequence
  Description of Artificial Sequence: Synthetic 6xHis tag
HHHHHH

SEQ ID NO: 39 - protein, 8 aa, Artificial Sequence
  Description of Artificial Sequence: Synthetic peptide
DYKDDDDK

SEQ ID NO: 40 - protein, 10 aa, Artificial Sequence
  Description of Artificial Sequence: Synthetic peptide
EQKLISEEDL

SEQ ID NO: 41 - protein, 9 aa, Artificial Sequence
  Description of Artificial Sequence: Synthetic peptide
YPYDVPDYA

SEQ ID NO: 42 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 232 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 47 - protein, 231 aa, Artificial Sequence
  Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 49 - protein, 216 aa, Artificial Sequence
  Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 50 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 52 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 53 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54-60 - skipped (7 entries)

SEQ ID NO: 61 - protein, 219 aa, Artificial Sequence
  Description of Artificial Sequence: Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - skipped

SEQ ID NO: 63 - protein, 226 aa, Artificial Sequence; identical to SEQ ID NO: 48
  Description of Artificial Sequence: Synthetic polypeptide

SEQ ID NO: 64-66 - skipped (3 entries)

SEQ ID NO: 67 - protein, 448 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 68 - protein, 449 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 69-75 - skipped (7 entries)

SEQ ID NO: 76 - protein, 8 aa, Artificial Sequence
  Description of Artificial Sequence: Synthetic peptide
GFTFSSFG

SEQ ID NO: 77 - protein, 8 aa, Artificial Sequence
  Description of Artificial Sequence: Synthetic peptide
GFTFSSYA

SEQ ID NO: 78 - protein, 8 aa, Artificial Sequence
  Description of Artificial Sequence: Synthetic peptide
GFIFSNYG

SEQ ID NO: 79 - protein, 8 aa, Artificial Sequence
  Description of Artificial Sequence: Synthetic peptide
GFTFSSSW

SEQ ID NO: 80 - protein, 8 aa, Artificial Sequence
  Description of Artificial Sequence: Synthetic peptide
GYAFSYSW

SEQ ID NO: 81 - protein, 10 aa, Artificial Sequence
  Description of Artificial Sequence: Synthetic peptide
GRTFTSYNMH

SEQ ID NO: 82 - protein, 8 aa, Artificial Sequence
  Description of Artificial Sequence: Synthetic peptide
GYTFTSYN

SEQ ID NO: 83 - protein, 8 aa, Artificial Sequence
  Description of Artificial Sequence: Synthetic peptide
GYTFTSYW

SEQ ID NO: 84 - protein, 8 aa, Artificial Sequence
  Description of Artificial Sequence: Synthetic peptide
GYSFTGYN

SEQ ID NO: 85 - protein, 8 aa, Artificial Sequence
  Description of Artificial Sequence: Synthetic peptide
GFTFNSFA

SEQ ID NO: 86 - protein, 8 aa, Artificial Sequence
  Description of Artificial Sequence: Synthetic peptide
GYTFTDYW

SEQ ID NO: 87 - protein, 8 aa, Artificial Sequence
  Description of Artificial Sequence: Synthetic peptide
GFKFSGYG

SEQ ID NO: 88 - protein, 8 aa, Artificial Sequence
  Description of Artificial Sequence: Synthetic peptide
GFTFTDFY

SEQ ID NO: 89 - protein, 8 aa, Artificial Sequence
  Description of Artificial Sequence: Synthetic peptide
GFTFSDAW

SEQ ID NO: 90 - protein, 8 aa, Artificial Sequence
  Description of Artificial Sequence: Synthetic peptide
GYIFTAYT

SEQ ID NO: 91 - protein, 8 aa, Artificial Sequence
  Description of Artificial Sequence: Synthetic peptide
GFTFSSYT

SEQ ID NO: 92 - protein, 8 aa, Artificial Sequence
  Description of Artificial Sequence: Synthetic peptide
GFNIKDTY

SEQ ID NO: 93 - protein, 8 aa, Artificial Sequence
  Description of Artificial Sequence: Synthetic peptide
GYTFRSSY

SEQ ID NO: 94 - protein, 8 aa, Artificial Sequence
  Description of Artificial Sequence: Synthetic peptide
GYTFTGYW

SEQ ID NO: 95 - protein, 8 aa, Artificial Sequence
  Description of Artificial Sequence: Synthetic peptide
GFAFSHYA

SEQ ID NO: 96 - protein, 8 aa, Artificial Sequence
  Description of Artificial Sequence: Synthetic peptide
GYTFTDYE

SEQ ID NO: 97 - protein, 8 aa, Artificial Sequence
  Description of Artificial Sequence: Synthetic peptide
GGTFSFYA

SEQ ID NO: 98 - protein, 8 aa, Artificial Sequence
  Description of Artificial Sequence: Synthetic peptide
GFSLSKFG

SEQ ID NO: 99 - protein, 8 aa, Artificial Sequence
  Description of Artificial Sequence: Synthetic peptide
GFTFKNYA

SEQ ID NO: 100 - protein, 8 aa, Artificial Sequence
  Description of Artificial Sequence: Synthetic peptide
GFDFSRYW

SEQ ID NO: 101 - protein, 8 aa, Artificial Sequence
  Description of Artificial Sequence: Synthetic peptide
GFTFNSYA

SEQ ID NO: 102 - protein, 8 aa, Artificial Sequence
  Description of Artificial Sequence: Synthetic peptide
GFTFSSYI

SEQ ID NO: 103 - protein, 5 aa, Artificial Sequence
  Description of Artificial Sequence: Synthetic peptide
SYWMH

SEQ ID NO: 104 - protein, 9 aa, Artificial Sequence
  Description of Artificial Sequence: Synthetic peptide
GYTFLNCPI

SEQ ID NO: 105 - protein, 10 aa, Artificial Sequence
  Description of Artificial Sequence: Synthetic peptide
GFTFSYYYMQ

SEQ ID NO: 106 - protein, 8 aa, Artificial Sequence
  Description of Artificial Sequence: Synthetic peptide
ISSDSSAI

SEQ ID NO: 107 - protein, 8 aa, Artificial Sequence
  Description of Artificial Sequence: Synthetic peptide
INASGTRT

SEQ ID NO: 108 - protein, 8 aa, Artificial Sequence
  Description of Artificial Sequence: Synthetic peptide
ISSASTYS

SEQ ID NO: 109 - protein, 8 aa, Artificial Sequence
  Description of Artificial Sequence: Synthetic peptide
IYPGDGDT

SEQ ID NO: 110 - protein, 8 aa, Artificial Sequence
  Description of Artificial Sequence: Synthetic peptide
IFPGDGDT

SEQ ID NO: 111 - protein, 9 aa, Artificial Sequence
  Description of Artificial Sequence: Synthetic peptide
AIYPLTGDT

SEQ ID NO: 112 - protein, 8 aa, Artificial Sequence
  Description of Artificial Sequence: Synthetic peptide
IYPGNGDT

SEQ ID NO: 113 - protein, 8 aa, Artificial Sequence
  Description of Artificial Sequence: Synthetic peptide
INPRNDYT

SEQ ID NO: 114 - protein, 8 aa, Artificial Sequence
  Description of Artificial Sequence: Synthetic peptide
IDPYYGGT

SEQ ID NO: 115 - protein, 8 aa, Artificial Sequence
  Description of Artificial Sequence: Synthetic peptide
ISGSGGGT

SEQ ID NO: 116 - protein, 8 aa, Artificial Sequence
  Description of Artificial Sequence: Synthetic peptide
IWYDGSKK

SEQ ID NO: 117 - protein, 10 aa, Artificial Sequence
  Description of Artificial Sequence: Synthetic peptide
IRDKAKGYTT

SEQ ID NO: 118 - protein, 10 aa, Artificial Sequence
  Description of Artificial Sequence: Synthetic peptide
IRSKASNHAT

SEQ ID NO: 119 - protein, 10 aa, Artificial Sequence
  Description of Artificial Sequence: Synthetic peptide
IRSANNHAPT

SEQ ID NO: 120 - protein, 8 aa, Artificial Sequence
  Description of Artificial Sequence: Synthetic peptide
IKPNNGLA

SEQ ID NO: 121 - protein, 8 aa, Artificial Sequence
  Description of Artificial Sequence: Synthetic peptide
ISYDGNNK

SEQ ID NO: 122 - protein, 8 aa, Artificial Sequence
  Description of Artificial Sequence: Synthetic peptide
IYPTNGYT

SEQ ID NO: 123 - protein, 8 aa, Artificial Sequence
  Description of Artificial Sequence: Synthetic peptide
IYAGTGSP

SEQ ID NO: 124 - protein, 8 aa, Artificial Sequence
  Description of Artificial Sequence: Synthetic peptide
IYPGSGNT

SEQ ID NO: 125 - protein, 8 aa, Artificial Sequence
  Description of Artificial Sequence: Synthetic peptide
ISSGGSGT

SEQ ID NO: 126 - protein, 8 aa, Artificial Sequence
  Description of Artificial Sequence: Synthetic peptide
LDPKTGDT

SEQ ID NO: 127 - protein, 8 aa, Artificial Sequence
  Description of Artificial Sequence: Synthetic peptide
FIPIFGAA

SEQ ID NO: 128 - protein, 7 aa, Artificial Sequence
  Description of Artificial Sequence: Synthetic peptide
IWGDGST

SEQ ID NO: 129 - protein, 8 aa, Artificial Sequence
  Description of Artificial Sequence: Synthetic peptide
IDPYYGDT

SEQ ID NO: 130 - protein, 8 aa, Artificial Sequence
  Description of Artificial Sequence: Synthetic peptide
ISYDGRNI

SEQ ID NO: 131 - protein, 8 aa, Artificial Sequence
  Description of Artificial Sequence: Synthetic peptide
INPDSSTI

SEQ ID NO: 132 - protein, 8 aa, Artificial Sequence
  Description of Artificial Sequence: Synthetic peptide
ISGSGGFT

SEQ ID NO: 133 - protein, 8 aa, Artificial Sequence
  Description of Artificial Sequence: Synthetic peptide
IYPSGGIT

SEQ ID NO: 134 - protein, 17 aa, Artificial Sequence
  Description of Artificial Sequence: Synthetic peptide
EIDPSDSYKDYNQKFKD

SEQ ID NO: 135 - protein, 11 aa, Artificial Sequence
  Description of Artificial Sequence: Synthetic peptide
GWMKPRGGAVN

SEQ ID NO: 136 - protein, 9 aa, Artificial Sequence
  Description of Artificial Sequence: Synthetic peptide
IGSSGGVTN

SEQ ID NO: 137 - protein, 15 aa, Artificial Sequence
  Description of Artificial Sequence: Synthetic peptide
GRGRENIYYGSRLDY

SEQ ID NO: 138 - protein, 19 aa, Artificial Sequence
  Description of Artificial Sequence: Synthetic peptide
ARGKGNTHKPYGYVRYFDV

SEQ ID NO: 139 - protein, 12 aa, Artificial Sequence
  Description of Artificial Sequence: Synthetic peptide
GRHSDGNFAFGY

SEQ ID NO: 140 - protein, 14 aa, Artificial Sequence
  Description of Artificial Sequence: Synthetic peptide
ARSGFITTVRDFDY

SEQ ID NO: 141 - protein, 12 aa, Artificial Sequence
  Description of Artificial Sequence: Synthetic peptide
ARNVFDGYWLVY

SEQ ID NO: 142 - protein, 14 aa, Artificial Sequence
  Description of Artificial Sequence: Synthetic peptide
ARSTYVGGDWQFDV

SEQ ID NO: 143 - protein, 15 aa, Artificial Sequence
  Description of Artificial Sequence: Synthetic peptide
CARSTYYGGDWYFNV

SEQ ID NO: 144 - protein, 11 aa, Artificial Sequence
  Description of Artificial Sequence: Synthetic peptide
ARYDYNYAMDY

SEQ ID NO: 145 - protein, 14 aa, Artificial Sequence
  Description of Artificial Sequence: Synthetic peptide
ARSTYYGGDWYFDV

SEQ ID NO: 146 - protein, 9 aa, Artificial Sequence
  Description of Artificial Sequence: Synthetic peptide
ARRDITTFY

SEQ ID NO: 147 - protein, 9 aa, Artificial Sequence
  Description of Artificial Sequence: Synthetic peptide
ARSVGPFDS

SEQ ID NO: 148 - protein, 15 aa, Artificial Sequence
  Description of Artificial Sequence: Synthetic peptide
AKDKILWFGEPVFDY

SEQ ID NO: 149 - protein, 13 aa, Artificial Sequence
  Description of Artificial Sequence: Synthetic peptide
ARGDYYGSNSLDY

SEQ ID NO: 150 - protein, 12 aa, Artificial Sequence
  Description of Artificial Sequence: Synthetic peptide
ARQMGYWHFDLW

SEQ ID NO: 151 - protein, 12 aa, Artificial Sequence
  Description of Artificial Sequence: Synthetic peptide
AREGHTAAPFDY

SEQ ID NO: 152 - protein, 9 aa, Artificial Sequence
  Description of Artificial Sequence: Synthetic peptide
TRWRRFFDS

SEQ ID NO: 153 - protein, 8 aa, Artificial Sequence
  Description of Artificial Sequence: Synthetic peptide
TRDSTATH

SEQ ID NO: 154 - protein, 11 aa, Artificial Sequence
  Description of Artificial Sequence: Synthetic peptide
ARSEITTEFDY

SEQ ID NO: 155 - protein, 11 aa, Artificial Sequence
  Description of Artificial Sequence: Synthetic peptide
ARTGWLGPFDY

SEQ ID NO: 156 - protein, 13 aa, Artificial Sequence
  Description of Artificial Sequence: Synthetic peptide
SRWGGDGFYAMDY

SEQ ID NO: 157 - protein, 13 aa, Artificial Sequence
  Description of Artificial Sequence: Synthetic peptide
ARHRDYYSNSLTY

SEQ ID NO: 158 - protein, 11 aa, Artificial Sequence
  Description of Artificial Sequence: Synthetic peptide
ARGGYYEDFDS

SEQ ID NO: 159 - protein, 12 aa, Artificial Sequence
  Description of Artificial Sequence: Synthetic peptide
TRVKLGTYYFDS

SEQ ID NO: 160 - protein, 8 aa, Artificial Sequence
  Description of Artificial Sequence: Synthetic peptide
TRFYSYTY

SEQ ID NO: 161 - protein, 16 aa, Artificial Sequence
  Description of Artificial Sequence: Synthetic peptide
ARIPSGSYYYDYDMDV

SEQ ID NO: 162 - protein, 7 aa, Artificial Sequence
  Description of Artificial Sequence: Synthetic peptide
VKPGGDY

SEQ ID NO: 163 - protein, 13 aa, Artificial Sequence
  Description of Artificial Sequence: Synthetic peptide
VKGGYYGHWYFDV

SEQ ID NO: 164 - protein, 19 aa, Artificial Sequence
  Description of Artificial Sequence: Synthetic peptide
ARPVRSRWLQLGLEDAFHI

SEQ ID NO: 165 - protein, 12 aa, Artificial Sequence
  Description of Artificial Sequence: Synthetic peptide
ARPDGNYWYFDV

SEQ ID NO: 166 - protein, 13 aa, Artificial Sequence
  Description of Artificial Sequence: Synthetic peptide
AKDRLVAPGTFDY

SEQ ID NO: 167 - protein, 13 aa, Artificial Sequence
  Description of Artificial Sequence: Synthetic peptide
ARIKLGTVTTVDY

SEQ ID NO: 168 - protein, 9 aa, Artificial Sequence
  Description of Artificial Sequence: Synthetic peptide
AITTTPFDF

SEQ ID NO: 169 - protein, 14 aa, Artificial Sequence
  Description of Artificial Sequence: Synthetic peptide
ARYFFGSSPNWYFD

SEQ ID NO: 170 - protein, 14 aa, Artificial Sequence
  Description of Artificial Sequence: Synthetic peptide
ARVGLGDAFDIWQQ

SEQ ID NO: 171 - protein, 6 aa, Artificial Sequence
  Description of Artificial Sequence: Synthetic peptide
QNVDTN

SEQ ID NO: 172 - protein, 7 aa, Artificial Sequence
  Description of Artificial Sequence: Synthetic peptide
QSVSSSY

SEQ ID NO: 173 - protein, 11 aa, Artificial Sequence
  Description of Artificial Sequence: Synthetic peptide
RNIVHINGDTY

SEQ ID NO: 174 - protein, 10 aa, Artificial Sequence
  Description of Artificial Sequence: Synthetic peptide
ESVDTFGISF

SEQ ID NO: 175 - protein, 11 aa, Artificial Sequence
  Description of Artificial Sequence: Synthetic peptide
KSLLHSNGITY

SEQ ID NO: 176 - protein, 5 aa, Artificial Sequence
  Description of Artificial Sequence: Synthetic peptide
SSVPY

SEQ ID NO: 177 - protein, 5 aa, Artificial Sequence
  Description of Artificial Sequence: Synthetic peptide
SSVSY

SEQ ID NO: 178 - protein, 12 aa, Artificial Sequence
  Description of Artificial Sequence: Synthetic peptide
QSVLYSANHKNY

SEQ ID NO: 179 - protein, 6 aa, Artificial Sequence
  Description of Artificial Sequence: Synthetic peptide
ENVYSY

SEQ ID NO: 180 - protein, 6 aa, Artificial Sequence
  Description of Artificial Sequence: Synthetic peptide
QSVSSY

SEQ ID NO: 181 - protein, 6 aa, Artificial Sequence
  Description of Artificial Sequence: Synthetic peptide
QDVSTV

SEQ ID NO: 182 - protein, 6 aa, Artificial Sequence
  Description of Artificial Sequence: Synthetic peptide
QNIDKY

SEQ ID NO: 183 - protein, 6 aa, Artificial Sequence
  Description of Artificial Sequence: Synthetic peptide
QSVIND

SEQ ID NO: 184 - protein, 10 aa, Artificial Sequence
  Description of Artificial Sequence: Synthetic peptide
ESVDSYANSF

SEQ ID NO: 185 - protein, 7 aa, Artificial Sequence
  Description of Artificial Sequence: Synthetic peptide
QSVGSSY

SEQ ID NO: 186 - protein, 6 aa, Artificial Sequence
  Description of Artificial Sequence: Synthetic peptide
QDVNTA

SEQ ID NO: 187 - protein, 12 aa, Artificial Sequence
  Description of Artificial Sequence: Synthetic peptide
QSVLNSGNQKNY

SEQ ID NO: 188 - protein, 6 aa, Artificial Sequence
  Description of Artificial Sequence: Synthetic peptide
QGIISY

SEQ ID NO: 189 - protein, 6 aa, Artificial Sequence
  Description of Artificial Sequence: Synthetic peptide
QDISNY

SEQ ID NO: 190 - protein, 11 aa, Artificial Sequence
  Description of Artificial Sequence: Synthetic peptide
QSLVHSNRNTY

SEQ ID NO: 191 - protein, 5 aa, Artificial Sequence
  Description of Artificial Sequence: Synthetic peptide
SSISY

SEQ ID NO: 192 - protein, 6 aa, Artificial Sequence
  Description of Artificial Sequence: Synthetic peptide
QDIGSS

SEQ ID NO: 193 - protein, 6 aa, Artificial Sequence
  Description of Artificial Sequence: Synthetic peptide
QDIRNY

SEQ ID NO: 194 - protein, 6 aa, Artificial Sequence
  Description of Artificial Sequence: Synthetic peptide
QDVGIA

SEQ ID NO: 195 - protein, 6 aa, Artificial Sequence
  Description of Artificial Sequence: Synthetic peptide
QGISSW

SEQ ID NO: 196 - protein, 9 aa, Artificial Sequence
  Description of Artificial Sequence: Synthetic peptide
SSDVGGYNY

SEQ ID NO: 197 - protein, 11 aa, Artificial Sequence
  Description of Artificial Sequence: Synthetic peptide
RASQSISNNLH

SEQ ID NO: 198 - protein, 8 aa, Artificial Sequence
  Description of Artificial Sequence: Synthetic peptide
SQYGSLAW

SEQ ID NO: 199 - protein, 8 aa, Artificial Sequence
  Description of Artificial Sequence: Synthetic peptide
SLSNIGLN

SEQ ID NO: 200 - protein, 6 aa, Artificial Sequence
  Description of Artificial Sequence: Synthetic peptide
YSQSIS

SEQ ID NO: 201 - protein, 9 aa, Artificial Sequence
  Description of Artificial Sequence: Synthetic peptide
QQYNNYPFT

SEQ ID NO: 202 - protein, 9 aa, Artificial Sequence
  Description of Artificial Sequence: Synthetic peptide
LQIYNMPIT

SEQ ID NO: 203 - protein, 9 aa, Artificial Sequence
  Description of Artificial Sequence: Synthetic peptide
FQGSLLPWT

SEQ ID NO: 204 - protein, 9 aa, Artificial Sequence
  Description of Artificial Sequence: Synthetic peptide
QQSKEVPFT

SEQ ID NO: 205 - protein, 9 aa, Artificial Sequence
  Description of Artificial Sequence: Synthetic peptide
AQNLELPYT

SEQ ID NO: 206 - protein, 9 aa, Artificial Sequence
  Description of Artificial Sequence: Synthetic peptide
QQWLSNPPT

SEQ ID NO: 207 - protein, 9 aa, Artificial Sequence
  Description of Artificial Sequence: Synthetic peptide
QQWTSNPPT

SEQ ID NO: 208 - protein, 9 aa, Artificial Sequence
  Description of Artificial Sequence: Synthetic peptide
QQWTFNPPT

SEQ ID NO: 209 - protein, 6 aa, Artificial Sequence
  Description of Artificial Sequence: Synthetic peptide
HQYLSS

SEQ ID NO: 210 - protein, 9 aa, Artificial Sequence
  Description of Artificial Sequence: Synthetic peptide
QHHSDNPWT

SEQ ID NO: 211 - protein, 9 aa, Artificial Sequence
  Description of Artificial Sequence: Synthetic peptide
QQRSNWPPT

SEQ ID NO: 212 - protein, 9 aa, Artificial Sequence
  Description of Artificial Sequence: Synthetic peptide
QQHYSPPYT

SEQ ID NO: 213 - protein, 10 aa, Artificial Sequence
  Description of Artificial Sequence: Synthetic peptide
QQRSNWPPLT

SEQ ID NO: 214 - protein, 9 aa, Artificial Sequence
  Description of Artificial Sequence: Synthetic peptide
LQHISRPRT

SEQ ID NO: 215 - protein, 9 aa, Artificial Sequence
  Description of Artificial Sequence: Synthetic peptide
QQWSSNPLT

SEQ ID NO: 216 - protein, 7 aa, Artificial Sequence
  Description of Artificial Sequence: Synthetic peptide
QQDTSPP

SEQ ID NO: 217 - protein, 9 aa, Artificial Sequence
  Description of Artificial Sequence: Synthetic peptide
QQSKEDPLT

SEQ ID NO: 218 - protein, 9 aa, Artificial Sequence
  Description of Artificial Sequence: Synthetic peptide
QQYGSSPWT

SEQ ID NO: 219 - protein, 9 aa, Artificial Sequence
  Description of Artificial Sequence: Synthetic peptide
QQHYTTPPT

SEQ ID NO: 220 - protein, 9 aa, Artificial Sequence
  Description of Artificial Sequence: Synthetic peptide
QSDYSYPYT

SEQ ID NO: 221 - protein, 9 aa, Artificial Sequence
  Description of Artificial Sequence: Synthetic peptide
GQYANYPYT

SEQ ID NO: 222 - protein, 7 aa, Artificial Sequence
  Description of Artificial Sequence: Synthetic peptide
HQYSKLP

SEQ ID NO: 223 - protein, 9 aa, Artificial Sequence
  Description of Artificial Sequence: Synthetic peptide
SQNTHVPPT

SEQ ID NO: 224 - protein, 9 aa, Artificial Sequence
  Description of Artificial Sequence: Synthetic peptide
QQRSNWMYT

SEQ ID NO: 225 - protein, 9 aa, Artificial Sequence
  Description of Artificial Sequence: Synthetic peptide
HQRDSYPWT

SEQ ID NO: 226 - protein, 9 aa, Artificial Sequence
  Description of Artificial Sequence: Synthetic peptide
LQYVSSPPT

SEQ ID NO: 227 - protein, 9 aa, Artificial Sequence
  Description of Artificial Sequence: Synthetic peptide
QQYYNSPPT

SEQ ID NO: 228 - protein, 9 aa, Artificial Sequence
  Description of Artificial Sequence: Synthetic peptide
QQYSSYPYT

SEQ ID NO: 229 - protein, 9 aa, Artificial Sequence
  Description of Artificial Sequence: Synthetic peptide
QQYNSYPYT

SEQ ID NO: 230 - protein, 10 aa, Artificial Sequence
  Description of Artificial Sequence: Synthetic peptide
SSYTSSSTRV

SEQ ID NO: 231 - protein, 9 aa, Artificial Sequence
  Description of Artificial Sequence: Synthetic peptide
QQSNTWPYT

SEQ ID NO: 232 - protein, 10 aa, Artificial Sequence
  Description of Artificial Sequence: Synthetic peptide
QQYEFFGQGT

SEQ ID NO: 233 - protein, 9 aa, Artificial Sequence
  Description of Artificial Sequence: Synthetic peptide
AAWDDSPPG

SEQ ID NO: 234-235 - skipped (2 entries)

SEQ ID NO: 236 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 237-238 - skipped (2 entries)

SEQ ID NO: 239 - protein, 943 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 240 - protein, 944 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 241 - protein, 721 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 242 - skipped

SEQ ID NO: 243 - protein, 696 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 244-246 - skipped (3 entries)

SEQ ID NO: 247 - protein, 448 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 248 - protein, 449 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 249 - protein, 32 aa, Artificial Sequence
  Description of Artificial Sequence: Synthetic polypeptide
GGSGGGSGGGSGGGGGSGGGGSGGGGSGGGGS

SEQ ID NO: 250 - protein, 30 aa, Artificial Sequence
  Description of Artificial Sequence: Synthetic polypeptide
  MISC_FEATURE (1)..(30): This sequence may encompass 4-30 residues
GGGGGGGGGGGGGGGGGGGGGGGGGGGGGG

SEQ ID NO: 251 - protein, 20 aa, Artificial Sequence
  Description of Artificial Sequence: Synthetic peptide
  MISC_FEATURE (1)..(20): This sequence may encompass 4-20 residues
GGGGGGGGGGGGGGGGGGGG

SEQ ID NO: 252 - protein, 30 aa, Artificial Sequence; identical to SEQ ID NO: 250
  Description of Artificial Sequence: Synthetic polypeptide
  MISC_FEATURE (1)..(30): This sequence may encompass 8-30 residues

SEQ ID NO: 253 - protein, 20 aa, Artificial Sequence
  Description of Artificial Sequence: Synthetic peptide
  MISC_FEATURE (1)..(20): This sequence may encompass 8-20 residues
GGGGGGGGGGGGGGGGGGGG

SEQ ID NO: 254 - protein, 20 aa, Artificial Sequence
  Description of Artificial Sequence: Synthetic peptide
  MISC_FEATURE (1)..(20): This sequence may encompass 12-20 residues
GGGGGGGGGGGGGGGGGGGG

SEQ ID NO: 255 - protein, 30 aa, Artificial Sequence; identical to SEQ ID NO: 250
  Description of Artificial Sequence: Synthetic polypeptide
  MISC_FEATURE (1)..(30): This sequence may encompass 12-30 residues

SEQ ID NO: 256 - protein, 20 aa, Artificial Sequence
  Description of Artificial Sequence: Synthetic peptide
EPKSCDKTHTCPPCPAPELL

SEQ ID NO: 257 - protein, 15 aa, Artificial Sequence
  Description of Artificial Sequence: Synthetic peptide
DKTHTCPPCPAPELL

SEQ ID NO: 258 - protein, 19 aa, Artificial Sequence
  Description of Artificial Sequence: Synthetic peptide
EPKSCDKTHTCPPCPAPEL

SEQ ID NO: 259 - protein, 105 aa, Artificial Sequence
  Description of Artificial Sequence: Synthetic polypeptide
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 260 - protein, 106 aa, Artificial Sequence [sequence omitted]

SEQ ID NO: 261 - protein, 447 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 451 aa, Artificial Sequence
  Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 451 aa, Artificial Sequence
  Description of Artificial Sequence: Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 264 - protein, 450 aa, Artificial Sequence
  Description of Artificial Sequence: Synthetic polypeptide
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 118 aa, Artificial Sequence
  Description of Artificial Sequence: Synthetic polypeptide
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTV

SEQ ID NO: 266 - protein, 214 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 215 aa, Artificial Sequence
  Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 214 aa, Artificial Sequence
  Description of Artificial Sequence: Synthetic polypeptide
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 214 aa, Artificial Sequence
  Description of Artificial Sequence: Synthetic polypeptide
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 109 aa, Artificial Sequence
  Description of Artificial Sequence: Synthetic polypeptide
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 271 - protein, 200 aa, Artificial Sequence
  Description of Artificial Sequence: Synthetic polypeptide
  MISC_FEATURE (1)..(200): This sequence may encompass 4-200 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 272 - protein, 180 aa, Artificial Sequence
  Description of Artificial Sequence: Synthetic polypeptide
  MISC_FEATURE (1)..(180): This sequence may encompass 4-180 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 273 - protein, 160 aa, Artificial Sequence
  Description of Artificial Sequence: Synthetic polypeptide
  MISC_FEATURE (1)..(160): This sequence may encompass 4-160 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 274 - protein, 140 aa, Artificial Sequence
  Description of Artificial Sequence: Synthetic polypeptide
  MISC_FEATURE (1)..(140): This sequence may encompass 4-140 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 275 - protein, 40 aa, Artificial Sequence
  Description of Artificial Sequence: Synthetic polypeptide
  MISC_FEATURE (1)..(40): This sequence may encompass 4-40 residues
GGGGGGGGGGGGGGGGGGGGGGGGGGGGGGGGGGGGGGGG

SEQ ID NO: 276 - protein, 100 aa, Artificial Sequence
  Description of Artificial Sequence: Synthetic polypeptide
  MISC_FEATURE (1)..(100): This sequence may encompass 4-100 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 277 - protein, 90 aa, Artificial Sequence
  Description of Artificial Sequence: Synthetic polypeptide
  MISC_FEATURE (1)..(90): This sequence may encompass 4-90 residues
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 278 - protein, 80 aa, Artificial Sequence
  Description of Artificial Sequence: Synthetic polypeptide
  MISC_FEATURE (1)..(80): This sequence may encompass 4-80 residues
GGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 279 - protein, 70 aa, Artificial Sequence
  Description of Artificial Sequence: Synthetic polypeptide
  MISC_FEATURE (1)..(70): This sequence may encompass 4-70 residues
GGGGGGGGGGGGGGGGGGGGGGGGGGGGGGGGGGGGGGGGGGGGGGGGGGGGGGGGGGGGGGGGGGGGGG

SEQ ID NO: 280 - protein, 60 aa, Artificial Sequence
  Description of Artificial Sequence: Synthetic polypeptide
  MISC_FEATURE (1)..(60): This sequence may encompass 4-60 residues
GGGGGGGGGGGGGGGGGGGGGGGGGGGGGGGGGGGGGGGGGGGGGGGGGGGGGGGGGGGG

SEQ ID NO: 281 - protein, 50 aa, Artificial Sequence
  Description of Artificial Sequence: Synthetic polypeptide
  MISC_FEATURE (1)..(50): This sequence may encompass 4-50 residues
GGGGGGGGGGGGGGGGGGGGGGGGGGGGGGGGGGGGGGGGGGGGGGGGGG

SEQ ID NO: 282 - protein, 19 aa, Artificial Sequence
  Description of Artificial Sequence: Synthetic peptide
  MISC_FEATURE (1)..(19): This sequence may encompass 4-19 residues
GGGGGGGGGGGGGGGGGGG

SEQ ID NO: 283 - protein, 18 aa, Artificial Sequence
  Description of Artificial Sequence: Synthetic peptide
  MISC_FEATURE (1)..(18): This sequence may encompass 4-18 residues
GGGGGGGGGGGGGGGGGG

SEQ ID NO: 284 - protein, 17 aa, Artificial Sequence
  Description of Artificial Sequence: Synthetic peptide
  MISC_FEATURE (1)..(17): This sequence may encompass 4-17 residues
GGGGGGGGGGGGGGGGG

SEQ ID NO: 285 - protein, 16 aa, Artificial Sequence
  Description of Artificial Sequence: Synthetic peptide
  MISC_FEATURE (1)..(16): This sequence may encompass 4-16 residues
GGGGGGGGGGGGGGGG

SEQ ID NO: 286 - protein, 15 aa, Artificial Sequence
  Description of Artificial Sequence: Synthetic peptide
  MISC_FEATURE (1)..(15): This sequence may encompass 4-15 residues
GGGGGGGGGGGGGGG

SEQ ID NO: 287 - protein, 14 aa, Artificial Sequence
  Description of Artificial Sequence: Synthetic peptide
  MISC_FEATURE (1)..(14): This sequence may encompass 4-14 residues
GGGGGGGGGGGGGG

SEQ ID NO: 288 - protein, 13 aa, Artificial Sequence
  Description of Artificial Sequence: Synthetic peptide
  MISC_FEATURE (1)..(13): This sequence may encompass 4-13 residues
GGGGGGGGGGGGG

SEQ ID NO: 289 - protein, 12 aa, Artificial Sequence
  Description of Artificial Sequence: Synthetic peptide
  MISC_FEATURE (1)..(12): This sequence may encompass 4-12 residues
GGGGGGGGGGGG

SEQ ID NO: 290 - protein, 11 aa, Artificial Sequence
  Description of Artificial Sequence: Synthetic peptide
  MISC_FEATURE (1)..(11): This sequence may encompass 4-11 residues
GGGGGGGGGGG

SEQ ID NO: 291 - protein, 10 aa, Artificial Sequence
  Description of Artificial Sequence: Synthetic peptide
  MISC_FEATURE (1)..(10): This sequence may encompass 4-10 residues
GGGGGGGGGG

SEQ ID NO: 292 - protein, 9 aa, Artificial Sequence
  Description of Artificial Sequence: Synthetic peptide
  MISC_FEATURE (1)..(9): This sequence may encompass 4-9 residues
GGGGGGGGG

SEQ ID NO: 293 - protein, 8 aa, Artificial Sequence
  Description of Artificial Sequence: Synthetic peptide
  MISC_FEATURE (1)..(8): This sequence may encompass 4-8 residues
GGGGGGGG

SEQ ID NO: 294 - protein, 7 aa, Artificial Sequence
  Description of Artificial Sequence: Synthetic peptide
  MISC_FEATURE (1)..(7): This sequence may encompass 4-7 residues
GGGGGGG

SEQ ID NO: 295 - protein, 6 aa, Artificial Sequence
  Description of Artificial Sequence: Synthetic peptide
  MISC_FEATURE (1)..(6): This sequence may encompass 4-6 residues
GGGGGG

SEQ ID NO: 296 - protein, 5 aa, Artificial Sequence
  Description of Artificial Sequence: Synthetic peptide
  MISC_FEATURE (1)..(5): This sequence may encompass 4-5 residues
GGGGG

SEQ ID NO: 297 - protein, 200 aa, Artificial Sequence; identical to SEQ ID NO: 271
  Description of Artificial Sequence: Synthetic polypeptide
  MISC_FEATURE (1)..(200): This sequence may encompass 6-200 residues

SEQ ID NO: 298 - protein, 200 aa, Artificial Sequence; identical to SEQ ID NO: 271
  Description of Artificial Sequence: Synthetic polypeptide
  MISC_FEATURE (1)..(200): This sequence may encompass 8-200 residues

SEQ ID NO: 299 - protein, 200 aa, Artificial Sequence; identical to SEQ ID NO: 271
  Description of Artificial Sequence: Synthetic polypeptide
  MISC_FEATURE (1)..(200): This sequence may encompass 10-200 residues

SEQ ID NO: 300 - protein, 200 aa, Artificial Sequence; identical to SEQ ID NO: 271
  Description of Artificial Sequence: Synthetic polypeptide
  MISC_FEATURE (1)..(200): This sequence may encompass 12-200 residues

SEQ ID NO: 301 - protein, 200 aa, Artificial Sequence; identical to SEQ ID NO: 271
  Description of Artificial Sequence: Synthetic polypeptide
  MISC_FEATURE (1)..(200): This sequence may encompass 14-200 residues

SEQ ID NO: 302 - protein, 200 aa, Artificial Sequence; identical to SEQ ID NO: 271
  Description of Artificial Sequence: Synthetic polypeptide
  MISC_FEATURE (1)..(200): This sequence may encompass 16-200 residues

SEQ ID NO: 303 - protein, 200 aa, Artificial Sequence; identical to SEQ ID NO: 271
  Description of Artificial Sequence: Synthetic polypeptide
  MISC_FEATURE (1)..(200): This sequence may encompass 18-200 residues

SEQ ID NO: 304 - protein, 200 aa, Artificial Sequence; identical to SEQ ID NO: 271
  Description of Artificial Sequence: Synthetic polypeptide
  MISC_FEATURE (1)..(200): This sequence may encompass 20-200 residues

SEQ ID NO: 305 - protein, 200 aa, Artificial Sequence; identical to SEQ ID NO: 271
  Description of Artificial Sequence: Synthetic polypeptide
  MISC_FEATURE (1)..(200): This sequence may encompass 30-200 residues

SEQ ID NO: 306 - protein, 200 aa, Artificial Sequence; identical to SEQ ID NO: 271
  Description of Artificial Sequence: Synthetic polypeptide
  MISC_FEATURE (1)..(200): This sequence may encompass 40-200 residues

SEQ ID NO: 307 - protein, 200 aa, Artificial Sequence; identical to SEQ ID NO: 271
  Description of Artificial Sequence: Synthetic polypeptide
  MISC_FEATURE (1)..(200): This sequence may encompass 50-200 residues

SEQ ID NO: 308 - protein, 200 aa, Artificial Sequence; identical to SEQ ID NO: 271
  Description of Artificial Sequence: Synthetic polypeptide
  MISC_FEATURE (1)..(200): This sequence may encompass 60-200 residues

SEQ ID NO: 309 - protein, 200 aa, Artificial Sequence; identical to SEQ ID NO: 271
  Description of Artificial Sequence: Synthetic polypeptide
  MISC_FEATURE (1)..(200): This sequence may encompass 70-200 residues

SEQ ID NO: 310 - protein, 200 aa, Artificial Sequence; identical to SEQ ID NO: 271
  Description of Artificial Sequence: Synthetic polypeptide
  MISC_FEATURE (1)..(200): This sequence may encompass 80-200 residues

SEQ ID NO: 311 - protein, 200 aa, Artificial Sequence; identical to SEQ ID NO: 271
  Description of Artificial Sequence: Synthetic polypeptide
  MISC_FEATURE (1)..(200): This sequence may encompass 90-200 residues

SEQ ID NO: 312 - protein, 200 aa, Artificial Sequence; identical to SEQ ID NO: 271
  Description of Artificial Sequence: Synthetic polypeptide
  MISC_FEATURE (1)..(200): This sequence may encompass 100-200 residues

SEQ ID NO: 313 - protein, 200 aa, Artificial Sequence; identical to SEQ ID NO: 271
  Description of Artificial Sequence: Synthetic polypeptide
  MISC_FEATURE (1)..(200): This sequence may encompass 120-200 residues

SEQ ID NO: 314 - protein, 200 aa, Artificial Sequence; identical to SEQ ID NO: 271
  Description of Artificial Sequence: Synthetic polypeptide
  MISC_FEATURE (1)..(200): This sequence may encompass 140-200 residues

SEQ ID NO: 315 - protein, 200 aa, Artificial Sequence; identical to SEQ ID NO: 271
  Description of Artificial Sequence: Synthetic polypeptide
  MISC_FEATURE (1)..(200): This sequence may encompass 160-200 residues

SEQ ID NO: 316 - protein, 200 aa, Artificial Sequence; identical to SEQ ID NO: 271
  Description of Artificial Sequence: Synthetic polypeptide
  MISC_FEATURE (1)..(200): This sequence may encompass 180-200 residues

SEQ ID NO: 317 - protein, 200 aa, Artificial Sequence; identical to SEQ ID NO: 271
  Description of Artificial Sequence: Synthetic polypeptide
  MISC_FEATURE (1)..(200): This sequence may encompass 190-200 residues

SEQ ID NO: 318 - protein, 24 aa, Artificial Sequence
  Description of Artificial Sequence: Synthetic peptide
DYKDDDDKDYKDDDDKDYKDDDDK

SEQ ID NO: 319 - protein, 30 aa, Artificial Sequence
  Description of Artificial Sequence: Synthetic polypeptide
EQKLISEEDLEQKLISEEDLEQKLISEEDL

SEQ ID NO: 320 - protein, 27 aa, Artificial Sequence
  Description of Artificial Sequence: Synthetic peptide
YPYDVPDYAYPYDVPDYAYPYDVPDYA

SEQ ID NO: 321 - protein, 696 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 322 - protein, 473 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 323 - protein, 474 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 324 - protein, 722 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQ

SEQ ID NO: 325 - protein, 696 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 326 - protein, 474 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGQKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 327 - protein, 473 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG